(19) United States
(12) Patent Application Publication
(10) Pub. No.: US 2026/0294834 A1
(43) Pub. Date: Oct. 1, 2026

(54) PRIMATE CARBONIC ANHYDRASE IV BINDING PEPTIDES AND AAVS

(71) Applicant: California lnstitute of Technology, Pasadena, CA (US)

(72) Inventors: Viviana Gradinaru, Pasadena, CA (US); Changfan Lin, Pasadena, CA (US); Xinhong Chen, Pasadena, CA (US); Timothy F. Shay, Service City, CA (US)

(21) Appl. No.: 19/479,968

(22) PCT Filed: May 15, 2024
(86) PCT No.: PCT/US2024/029396

§ 371 Date: Oct. 29, 2025

Related U.S. Application Data
(60) Provisional application No. 63/466,766, filed on May 16, 2023.

(51) Int. Cl. A61K 9/51; A61K 9/00; C07K 7/06; C07K 14/005

(52) U.S. Cl. CPC A61K 9/5184; A61K 9/0019; C07K 7/06; C07K 14/005; C07K 2319/00; C12N 2750/14122; C12N 2750/14142

(57) ABSTRACT

Disclosed herein include novel blood-brain barrier (BBB)-crossing receptors on the BBB interface, targeting peptides and derivatives thereof capable of binding to the novel receptors, and related methods of using the receptors to increase the permeability of the BBB and to deliver an agent to a nervous system (e g. CNS).

### 1. STATEMENT REGARDING FEDERALLY SPONSORED R&D

This invention was made with government support under Grant No. NS111369 awarded by National Institutes of Health. The government has certain rights in the invention.

### II. FIELD OF THE INVENTION

The present disclosure relates generally to the field of gene delivery. More specifically, methods and compositions are disclosed for crossing the blood brain barrier.

### III. REFERENCE TO SEQUENCE LISTING

The present application is being filed along with a Sequence Listing in electronic format. The Sequence Listing is provided as a file named RECE-004-01WO.xml, created on May 15, 2024, which is 3,502 kilobytes in size. The information in the electronic format of the Sequence Listing is incorporated herein by reference in its entirety.

### IV. BACKGROUND

Targeting therapeutic and research molecules to tissues and cell types of interest and away from tissues and cell types mediating potentially hazardous side effects is a foundational problem for drug development. This is especially the case for molecules targeting the brain. The blood-brain barrier (BBB) sharply limits the properties of therapeutic and research molecules that may enter the central nervous system (CNS) upon systemic administration into the peripheral bloodstream. The blood brain barrier (BBB) presents a fundamental bottleneck to the development of effective research tools and therapeutics for the central nervous system (CNS). This structure, comprising mainly of brain endothelial cells, requires large molecules to be delivered via invasive intracranial injections, technically challenging focused ultrasound, or receptor-mediated transcytosis. The rational design of BBB-crossing large molecules has long been hampered by the imperfect understanding of the mechanisms involved in transcytosis, with only a handful of targets, such as the transferrin receptor, validated for research and therapies.

Specifically, transporting therapeutic and diagnostic material (drugs, genes, antibodies, oligonucleotides) across the blood-brain barrier (BBB) into the central nervous system (CNS) and across the blood-retinal barrier into the eye poses a significant obstacle for addressing brain and eye diseases. Challis, R. C., Ravindra Kumar, S., Chen, X., Goertsen, D., Coughlin, G. M., Hori, A. M., Chuapoco, M. R., Otis, T. S., Miles, T. F., and Gradinaru, V. (2022). Adeno-Associated Virus Toolkit to Target Diverse Brain Cells. Annu Rev Neurosci 45, 447-469. 10.1146/annurev-neuro-111020-100834.

### V. SUMMARY OF THE INVENTION

There is a need for transporting therapeutic and diagnostic material (drugs, genes, antibodies, oligonucleotides) across the blood-brain barrier (BBB) into the central nervous system (CNS) and across the blood-retinal barrier into the eye poses a significant obstacle for addressing brain and eye diseases. The conventional methods require the administration of the molecules via intracranial injections for delivery of therapeutic targets to the cells.

Accordingly, in certain aspects, the invention provides methods of increasing permeability of the blood brain barrier. In some embodiments, the method comprises: providing a CA4 binding peptide (also referred to as “targeting peptide”) capable of binding to primate and/or human carbonic anhydrase 4 (CA4 or CA-IV), thereby increasing permeability of the blood brain barrier.

In certain aspects, the CA4 binding peptide is listed in Tables 1, 3, 4 and 5 In certain embodiments, the targeting peptides of the invention increase the permeability of the blood brain barrier is increased by at least 25%, 50%, 75%, 100%, or more as compared to the absence of the targeting peptide.

The invention further provides methods of delivering a payload to a nervous system of a subject. In some embodiments, the method comprises: providing a targeting peptide capable of binding to human CA4, primate CA4, or a derivative thereof, wherein the targeting peptide is part of a delivery system, and wherein the delivery system comprises the payload to be delivered to the nervous system; and administering the delivery system to the subject.

In some embodiments, the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and any combination thereof. In some embodiments, the delivery system comprises a viral vector or a non-viral vector.

In some embodiments, the CA4 binding peptide enhances the binding affinity of the viral vector or the non-viral vector to human CA4 or primate CA4. In some embodiments, the viral vector comprises an AAV vector In some embodiments, the targeting peptide is part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh 8, rhesus isolate rh.10, and a variant thereof. In some embodiments, the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

In some embodiments, the payload or therapeutic cargo to be delivered to a nervous system is a biological molecule, a non-biological molecule, or a combination thereof. In some embodiments, the biological molecule is selected from the group consisting of a nucleic acid sequence, a protein, a peptide, a lipid, a polysaccharide, and any combination thereof. In some embodiments, the payload is a therapeutic molecule. In some embodiments, the nucleic acid sequence to be delivered to a nervous system comprises one or more of: a) a sequence encoding a trophic factor, a growth factor, or other soluble factors that might be released from the transduced cells and affect the survival or function of that cell and/or surrounding cells; b) a DNA that restores protein function to humans or animals harboring a genetic mutation(s) in that gene; c) a DNA that encodes a protein that can be used to control or alter the activity or state of a cell, d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

The invention beneficially recognizes that systemic delivery of adeno-associated virus (AAVs), as opposed to direct brain or eye injection, offers a non-invasive approach with broader distribution, which is particularly beneficial for diffuse neurological disorders.

Accordingly, in certain aspects, the invention provides methods of increasing permeability of the blood brain barrier for delivery of therapeutic cargo across the BBB to the central nervous system and/or eye. In certain embodiments, the invention provides AAVs binding to rhesus CA4 (rhCA4) or human CA4 (huCA4) for CNS therapeutic cargo delivery in these species for both preclinical and clinical applications. In other beneficial aspect of the invention, CA4 is also highly concentrated in the primate brain, eye, and gut compared to other reported transporters such as transferrin receptor with broader expression patterns. Thus, the methods provided in the invention for therapeutic cargo delivery across in the CNS may have reduced off-target organ delivery.

The AAVs are typically screened and developed using the mice CA4 to assess the BBB permeability of AAVs comprising capsids. Nonetheless, there are differences in the AAV binding pocket between murine CA4 and primate CA4. In particular, engineered msCar4-binding AAVs identified through directed evolution in mice without mechanistic insight, such as 9P31 or 9P36, do not bind to rhCA4 or huCA4. Thus, there is a need for identification of AAVs that are capable of binding to primate or human CA4 The invention recognizes that such AAVs would be able to deliver the therapeutic cargo across CNS in primates and/or humans.

Accordingly, in certain aspects, the invention provides CA4 binding peptides that bind to human CA4 and/or primate CA4. Unless specified otherwise, the CA4 binding peptides referred to in this application are peptides that bind to human CA4 and/or primate CA4. In certain embodiments, the CA4 binding peptides are inserted in capsid proteins of an AAV. In certain preferred embodiments, the CA4 binding peptide is inserted between two adjacent amino acids in AA587-594 of SEQ ID NO: 1 of the AAV9 vector or functional equivalents of AA587-594 in an amino acid sequence at least 80% identical to SEQ ID NO: 1. In some embodiments, the targeting peptide is inserted between AA588-589 of SEQ ID NO: 1 of the AAV9 vector or functional equivalents of AA588-589 in an amino acid sequence at least 80% identical to SEQ ID NO: 1. In certain embodiments, the CA4 binding peptide is listed in Tables 1, 3, 4, and 5.

In certain aspects, the invention provides AAV capsid proteins that bind to primate CA4 and/or human CA4. In certain embodiments, these AAV capsid proteins comprise an insertion of CA4 binding peptides. In certain embodiments, the AAV capsid proteins or portions thereof that bind to primate and/or human CA4 are listed in Tables 1, 3, 4, and 5.

In certain aspects, the invention provides AAVs comprising AAV capsid proteins that bind to primate CA4 and/or human CA4. In certain embodiments, these AAV capsid proteins comprise an insertion of CA4 binding peptides. In certain embodiments, the AAV capsid proteins or portions thereof that bind to primate and/or human CA4 are listed in Tables 1, 3, 4, and 5.

In certain aspects, the invention provides recombinant adeno-associated virus (rAAV). In some embodiments, the rAAV comprises any of the AAV capsid protein disclosed herein. In some embodiments, the rAAV comprises an AAV capsid protein which comprises a CA4 binding peptide having a binding specificity to human CA4 and/or primate CA4, wherein the amino acid sequence of the targeting peptide is inserted between two adjacent amino acids in AA587-594, or functional equivalents thereof, of the AAV9 capsid protein. In some embodiments, the two adjacent amino acids are AA588 and AA589. In some embodiments, the rAAV has enhanced tropism for the nervous system relative to an rAAV that does not comprise the targeting peptide. In some embodiments, the rAAV is capable of transducing the nervous system with an efficiency at least 2-fold higher than an rAAV that does not comprise the targeting peptide.

In certain embodiments, the AAVs or rAAVs of the invention are employed to traverse the BBB and transduce the CNS in primates efficiently using various non-invasive administration routes, including systemic administration.

In certain embodiments, the methods of the invention enable a more translatable application of these AAVs in preclinical testing and clinical intervention for neurological diseases. In addition, in certain embodiments, the peptide modifications of these AAVs are further recruited for non-viral delivery such as for antibodies, antibody-drug conjugates, oligonucleotides, enzymes, proteins, larger synthetic molecules, exosomes, nanoparticles, and contrast agents.

In certain aspects, the invention provides carbonic anhydrase IV (CA4)-binding peptides listed in Tables 1, 3, 4, and 5. In certain aspects, the invention provides AAVs listed in Tables 1, 3, 4, and 5. These CA4 binding peptides and AAVs, listed in Tables 1, 3, 4, and 5 provide genetic access to primate central nervous system following non-invasive systemic delivery. In certain embodiments, the invention provides the 9-amino acid peptides listed in Tables 4 and 5.

In certain embodiments, the invention provides AAVs provided in Tables 4 and 5. In certain embodiments, the AAVs comprise 7-amino acid peptide insertion at 588/589 site of AAV9 and AQ or DG at position 587 to 588.

In certain aspects, the invention provides huCA4-binding AAVs Alpha 43, 45, 48, and 49. In certain embodiments, the huCA4-binding AAVs binding AAVs peptides listed in Table 1 In certain preferred embodiments, the invention provides that huCA40binding AAV peptides: DGVVHETVR; DGVVGVNIR; DGEVGLTVR; DGIVGSTIR.

In certain preferred aspects, the invention provides methods of treatment or diagnostic analysis of conditions related to CNS and/or eye by administration compositions comprising the peptides or AAVs provided herein. In certain embodiments, the peptides and/or AAVs of the invention allow for the delivery of therapeutic cargo directed to treatment of CNS and/or eye across the BBB. In certain embodiments, the invention provides methods of treatment of brain disorders. In certain embodiments, the brain disorders treated by the compositions of the invention include brain cancer, neurodegeneration and glioblastoma. In certain embodiments, the condition associated with eye is glaucoma.

In certain embodiments, the invention provides compositions for use in the delivery of an agent to a nervous system of a subject in need. In some embodiments, the composition comprises an AAV comprising (1) an AAV capsid protein disclosed herein and (2) an agent to be delivered to the nervous system of the subject; optionally wherein the nervous system is the central nervous system (CNS), the peripheral nervous system (PNS), or a combination thereof. In some embodiments, the nervous system is brain endothelial cells, neurons, capillaries in the brain, arterioles in the brain, arteries in the brain, or a combination thereof. In some embodiments, the composition is a pharmaceutical composition comprising one or more pharmaceutical acceptable carriers. In some embodiments, the agent to be delivered comprises a nucleic acid, a peptide, a small molecule, an aptamer, or a combination thereof.

In certain aspects, the invention provides delivery systems for delivery of peptides in the CNS. In some embodiments, the delivery system comprises (1) a CA4 binding peptide having specificity to primate CA4 and/or human CA4; and (2) an agent. In some embodiments, the targeting peptide is (1) displayed on the surface of the delivery system; or (2) partially embedded in the delivery system. In some embodiments, the delivery system is selected from the group consisting of: nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a viral vector or a non-viral vector. In some embodiments, the delivery system comprises a nanoparticle selected from the group consisting of: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

In certain embodiments, the invention provides HA-tag modified huCA4 as a capture agent for resin-based screening for AAV/antibody engineering. In certain embodiments, the invention provides HA-tag modified rhCA4 as a capture agent for resin-based screening for AAV/antibody engineering. In certain embodiments, the rhCA4 sequences are provided in Table 2.

In certain aspects, the invention provides administration of composition comprising the peptides provided herein in a subject. In certain embodiments, the compositions of the invention are administered as systemic bloodstream delivery. In certain embodiments, the compositions of the invention are administered as local direct injections. In certain embodiments, the invention provides compositions comprising AAVs and/or peptides of the invention. In certain embodiments, the compositions are injectables. In certain embodiments, the compositions are ointments. In certain embodiments, the compositions are orally administered compositions. In certain embodiments, the compositions are orally administered compositions.

### VI. BRIEF DESCRIPTION OF THE DRAWINGS

FIG. 1 provides an overview of the process of the selection of the CA4 binding peptides of the invention

FIG. 2 provides the composition of unique CA4-binding AAVs of the invention.

FIG. 3 provides msCar4 resin-based selection with a low dose of library input (1.6e11 vg).

FIG. 4 provides msCar4 resin-based selection with a high dose of library input (8e11 vg).

FIG. 5 provides AAV variants performance in resin-based selection at low and high dose.

FIG. 6 provides performance of AAV variants in cell-based selection at varying doses.

FIG. 7 provides AAV variants performance in LY6A resin-based selection across low (sample 1, 1.6e11 vg) and high dose (sample 2, 8e11 vg).

FIG. 8 provides the library pools generated using the methods provided in the invention.

FIG. 9 provides data pertaining to huCA4-binding AAVs after the Round 2 selection at a low library input dose (5e10 vg).

FIG. 10 provides data pertaining to huCA4-binding AAVs after the Round 2 selection at a high library input dose (2.5e11 vg).

FIG. 11 provides data for independent round 1 selection replicate 2 for huCA4-binding AAVs at a low (sample 1, 1.6e11 vg) and high (sample 2, 8e11) library input dose.

FIG. 12 provides data for independent round 1 selection replicate 3 for huCA4-binding AAVs at a low (sample 1, 1.6e11 vg) and high (sample 2, 8e11) library input dose.

FIG. 13 provides round 2 pulldown selection results for rhCA4-binding AAVs at a low (sample 1, 5e10 vg) and high (sample 2, 2.5e11 vg) library input dose.

FIG. 14 provides independent round 1 selection replicate 2 for rhCA4-binding AAVs at a low (sample 1, 1.6e11 vg) and high (sample 2, 8e11) library input dose.

FIG. 15 provides independent round 1 selection replicate 2 for rhCA4-binding AAVs at a low (sample 1, 1.6e11 vg) and high (sample 2, 8e11) library input dose.

FIG. 16 provides data from round 2 pulldown selection outcomes for huCA4-binding AAVs at a low library input dose (5e10 vg).

FIG. 17 provides data from round 2 pulldown selection outcomes for huCA4-binding AAVs at a high library input dose (2.5e11 vg).

FIG. 18 provides data for Round 2 pulldown selection outcomes for rhCA4-binding AAVs at a low (sample 1, 5e10 vg) and high (sample 2, 2.5e11 vg) library input dose.

FIG. 19 provides data from pull-down assay evaluating individual AAV variants' binding with huCA4.

FIG. 20 provides the data for the assessment of huCA4-enhanced cell transduction of Alpha 43, 45, 48, and 49.

FIG. 21 provides pull-down assay evaluating individual AAV variants' binding with rhCA4.

FIG. 22 provides an overview of the mice with humanized CA4.

FIG. 23 provides data for huCA4-binding AAVs performance in mice.

FIG. 24 provides data for cell-based selection of huCA4-binding AAVs.

FIG. 25 provides data round 3 pulldown selection outcomes for huCA4-binding AAVs at a low (sample 1, 1.6e10 vg) and high (sample 2, 7.8e10 vg) library input dose.

FIG. 26 provides data for cell-based selection of rhCA4-binding AAVs in round 3.

FIG. 27 provides round 3 pulldown selection outcomes for rhCA4-binding AAVs at a low (sample 1, 1.6e10 vg) and high (sample 2, 7.8e10 vg) library input dose.

### VII. DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Blood-Brain Barrier and CNS

Transporting therapeutic and diagnostic material (drugs, genes, antibodies, oligonucleotides) across the blood-brain barrier (BBB) into the central nervous system (CNS) and across the blood-retinal barrier into the eye poses a significant obstacle for addressing brain and eye diseases.

Blood-brain barrier (BBB) has emerged as a complex, dynamic, adaptable interface that controls the exchange of substances between the central nervous system (CNS) and the blood, to prevent the uncontrolled leakage of substances from the blood into the brain. The cells that make up the structure of the BBB include mostly brain endothelial cells, which constantly communicate with the other cells of the CNS (e.g., astrocytes, microglia, neurons, mast cells and pericytes, as well as circulating immune cells), adapting their behaviors to serve the needs of the CNS, responding to pathological conditions, and in some cases participating in the onset, maintenance or progression of disease. The complexity of BBB functions explains much of the difficulty in developing drugs that can cross the BBB. Utilizing receptors on the BBB interface can offer a method of crossing BBB.

### Carbonic Anhydrase IV

Carbonic anhydrase IV is an isozyme that belongs to the carbonic anhydrase family, a family of zinc metalloenzymes, which catalyzes the reversible reaction of hydration of CO₂, allowing the enzyme to regulate intra- and extra-cellular concentrations of CO₂, H⁺, and HCO₃⁻ The carbonic anhydrases participate in a variety of biological processes, including respiration, calcification, acid-base balance, bone resorption, and the formation of aqueous humor, cerebrospinal fluid, saliva, and gastric acid. The carbonic anhydrases show extensive diversity in tissue distribution and in their subcellular localization. There are at least seven genetically distinct isozymes of mammalian carbonic anhydrase, designated I-VII, each of which catalyzes the reversible hydration of carbon dioxide by a zinc-hydroxide mechanism. Physiological functions that are regulated by carbonic anhydrase comprise, for example, removal of HCO₃⁻ in lung by respiration, reutilization of HCO₃⁻ in kidney, production of aqueous humor in eyes, cerebrospinal fluids in brain, gastric juice production in stomach, pancreatic juice, and bone resorption by osteo-clasts. Carbonic anhydrase family members also play important roles in metabolic processes that include ureagenesis, gluconeogenesis, and lipogenesis.

Different from other carbonic anhydrases that are either soluble or attached to the plasma membrane by a membrane-spanning domain, carbonic anhydrase IV (CA4) is a glycosylphosphatidyl-inositol-anchored membrane isozyme. Carbonic anhydrase IV is broadly conserved across vertebrates and has similar CNS expression profiles in humans, with a recent single cell analysis of human brain vasculature confirming CA4's expression in the human BBB. Carbonic anhydrase IV has been shown to regulate pH, which is associated with neural discharge and can influence neuronal function through ion-gated channels.

In some embodiments, the carbonic anhydrase IV disclosed herein is a human carbonic anhydrase IV (CA4). CA4 is known to localize on the luminal surface of brain endothelial cells throughout the cortex and cerebellum where it enzymatically modulates carbon dioxide-bicarbonate balance. Human CA4 has been previously characterized as a 35-kDa protein with a “high activity” in CO₂ hydration and a higher activity than other isozymes in catalyzing the dehydration of HCOs. In general, human CA4 contains an 18-amino acid signal sequence at the N-terminal of the protein for endoplasmic reticulum (ER) translocation and a 260-amino acid “CA domain” containing active site amino acid residues that shows 30-36% homology with cytoplasmic CAs. At the C-terminal, an additional 27 amino acid residues containing the hydrophobic sequences of 21 amino acids sufficient to span the membrane are preceded by the 6-amino acid signal sequence for GPI-anchoring. The amino acid residue, Ser 266, was identified as the site for the attachment of the GPI anchor. The removal of C-terminal hydrophobic domain found in the CA4 precursor has important impact on GPI-anchoring, cell surface expression, and realization of the enzyme activity. Based on amino acid sequences deduced from the nucleotide sequence, human CA4 contains no classical consensus sites (Asn-Xxx-Ser/Thr) for N-glycosylation. Human CA4 also contains no oligosaccharide chains, while other mammalian carbonic anhydrase IV (e.g. mouse carbonic anhydrases IV (Car4)) are glycoproteins with one to several oligosaccharide side chains.

In some embodiments, a carbonic anhydrase IV (Car4) disclosed herein as a receptor for enhancing BBB crossing can be any carbonic anhydrase IV, such as a mouse Car4, a human CA4, or a homology or a variant thereof. Carbonic anhydrase IV homologs and/or variants can be derived from a vertebrate species including, but not limited to, mouse, rat, human, bovine, rabbit, monkey, pig, horse, rainbow trout, chimpanzee, squirrel, chicken, goat, and sheep. Carbonic anhydrase IV homologs from various species can be found in public databases identifiable to a person skilled in the art, including for example UniProt, NCBI, and Swiss-Prot.

In certain embodiments, the sequences for human CA4 (huCA4) and rhesus CA4 (rhCA4) are provided in Table 2A. In certain embodiments, the carbonic anhydrase IV homologs and variants can be about or can be at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequences huCA4 and/or rhCA4 provided in Table 2A. In certain embodiments, the huCA4 is at least 75%, about 80%, about 85%, about 90%, about 95%, or about 98% identical to the sequence provided in SEQ ID NO: 1. In certain embodiments, the huCA4 is at least 75%, about 80%, about 85%, about 90%, about 95%, or about 98% identical to the sequence provided in SEQ ID NO: 2. In certain embodiments, the rhCA4 is at least 75%, about 80%, about 85%, about 90%, about 95%, or about 98% identical to the sequence provided in SEQ ID NO: 3. In certain embodiments, the rhCA4 is at least 75%, about 80%, about 85%, about 90%, about 95%, or about 98% identical to the sequence provided in SEQ ID NO: 4.

In some embodiments, a human CA4 disclosed herein as a receptor for enhancing BBB crossing comprises a sequence provided in WO 2023/168333, which is incorporated by reference in its entirety. In certain embodiments, the human CA4 receptor is provided in NCBI Reference Sequence NM_000717.5, which is incorporated by reference in its entirety.

### CREATE System

In some embodiments, the CA4 binding peptides of the present disclosure are isolated via the CREATE system, as described in Deverman et al., (Nature Biotechnology 34(2):204-209 (2016)) and in International Patent Application Publication Nos. WO2015038958 and WO2017100671, the contents of each of which are herein incorporated by reference in their entirety. “CREATE” or “Cre-recombinant-based AAV targeted evolution” refers to an AAV capsid selection strategy that selects for capsids that transduce target tissues (e.g., CNS or PNS) following intravenous injection. The method has been demonstrated in a mouse model.

Libraries of AAV capsids with one or more targeting peptide inserts are developed and administered intravenously to transgenic mice. These transgenic Cre-expressing mice may be developed for specific targeting, for example, GFAP-Cre mice may be used for targeting to astrocytes. In some embodiments, Cre/LoxP mediated system can be used to knock-out or over and/or ectopically express CA4 to identify targeting peptides that interact with CA4.

Variation of the targeting sequence as well as the transgenic animal model enables the selection of AAV variants with desired transduction profiles, for example, tropism to neurons or astrocytes, as compared to other AAV serotypes, including the parent AAV particle and capsid.

The CREATE method involves the generation of a library of targeting peptides which are then assembled into a viral genome backbone comprising a parent AAV capsid sequence. An AAV capsid library (AAV particles) is then generated, purified and administered to a transgenic animal (e.g., mouse). Target tissue is collected and AAV sequences selectively recovered from Cre expressing cells. These sequences are assessed and characterized for the identification of targeting peptides that lead to enrichment in a target tissue (i.e., enhanced transduction or tropism). Targeting peptides and associated AAV particles can then be generated for further testing and characterization. This process is considered one round of evolution or selection. In some embodiments, more than one round of evolution is conducted. As many as 15 rounds of selection may be conducted.

In more detail, the CREATE system uses an rAAV-Cap-in-cis-lox viral genome comprising AAV cap and regulator elements of the AAV rep genes and a Cre-invertible switch. Since this viral genome lacks a fully functioning rep gene necessary for AAV particle production, the rep is provided in trans. A modified AAV2/9 Rep-Cap plasmid may be provided, wherein stop-codons are provided in-frame to prevent the expression of VP1-VP3 proteins.

Capsid libraries are generated using the rAAV-Cap-in-cis-lox viral genome as a backbone. Targeting peptides are inserted into the parent AAV capsid protein (e.g., AAV9) at any position that results in the generation of a fully functional AAV capsid protein and AAV particle. Targeting peptides may be designed by any method known in the art. In some embodiments, targeting peptides are generated using polymerase chain reaction (PCR). AAV particles comprising capsid proteins with targeting peptide inserts are generated and viral genomes encoding a reporter (e.g., GFP) encapsulated within. These AAV particles (or AAV capsid library) are then administered to a transgenic mouse by intravenous delivery to the tail vein. Administration of these capsid libraries to Cre-expressing mice results in expression of the reporter payload in the target tissue, due to the expression of Cre.

AAV particles and/or viral genomes may be recovered from the target tissue for identification of targeting peptides and associated AAV particles that are enriched, indicating enhanced transduction of target tissue. Standard methods in the art, such as, but not limited to next generation sequencing (NGS), viral genome quantification, biochemical assays, immunohistochemistry and/or imaging of target tissue samples may be used to determine enrichment.

A target tissue may be any cell, tissue or organ of a subject. As non-limiting examples, samples may be collected from brain, spinal cord, dorsal root ganglia and associated roots, liver, heart, gastrocnemius muscle, soleus muscle, pancreas, kidney, spleen, lung, adrenal glands, stomach, sciatic nerve, saphenous nerve, thyroid gland, eyes (with or without optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, or being of interest.

Targeting peptides and associated AAV capsid proteins and AAV particles identified using a CREATE system include AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), and AAVPHP.S. In some embodiments, CREATE in mice is used to identify AAV capsids and/or targeting peptides having enhanced transduction of a target tissue (e.g., CNS or PNS).

The CREATE system has proven efficacious in identifying targeting peptides for enhanced transduction to the CNS of mice after intravenous administration. However, translation of findings from mouse to human is not always straightforward. Modifying the CREATE system for non-transgenic animals or model systems that more closely resemble humans may help identify targeting peptides and associated AAV capsids and particles useful for the treatment of human disease. In some embodiments, the AAV interactors identified herein can be used to address this unmet need and assess or generate new models for design of AAV capsids in humans.

For adaptation of the CREATE method to non-transgenic animals, another mechanism needs to be used to alter target tissues and/or cells to express Cre. In some embodiments, AAV Cre-vectors may be used to transduce cells and induce subsequent Cre expression. In some embodiments, these AAV Cre-vectors may be AAV1-Cre vectors. The AAV Cre-vectors can comprise viral genomes with a cell-type specific promoter. These cell-type specific promotors may be, but are not limited to, CAG, UBC, EF1α, synapsin, GFAP, MBP, VGLUT, VGAT, Nav1.8, parvalbumin, TH, ChaT, and/or any promoter known in the art.

In some embodiments, these AAV-Cre vectors are delivered to a target tissue by intraparenchymal administration. In some embodiments, the intraparenchymal administration is directly to the putamen of the subject. In some embodiments, the intraparenchymal administration is directly to the thalamus of a subject. In some embodiments, the intraparenchymal administration is directly to the cortex of a subject. In some embodiments, the intraparenchymal administration is indirectly to the cortex of a subject. In some embodiments, the intraparenchymal administration is simultaneously to one or more of the putamen, the thalamus and or the cortex of a subject, and may be bi-lateral administrations. In some embodiments, the subject is a non-human primate.

As for the CREATE method developed in mice, the AAV capsid libraries may be administered intravenously. In another embodiment, the AAV capsid libraries may be administered by intraparenchymal delivery. In some embodiments, the AAV capsid library is administered prior to the delivery of the AAV-Cre vectors. In another embodiment, the AAV capsid library is administered after the delivery of the AAV-Cre vectors. The length of time between the administration of the AAV-Cre vectors and the AAV capsid libraries may be seconds, minutes, hours, days, weeks, or years.

The AAV capsid library may comprise AAV particles comprising a viral genome encoding a reporter (e.g., GFP). Only those cells of the target tissue (e.g., CNS or DRG) also expressing Cre (co-transduced by a Cre-vector administered intraparenchymally) will express the reporter. Target tissues may be collected and analyzed for the identification of AAV particles and targeting peptides that lead to enrichment in the target tissue, i.e., enhanced transduction. Standard methods in the art may be used to assess, analyze, or characterize sample tissues and AAV sequences, including but not limited to, next generation sequencing, viral genome quantification, biochemical assays, immunohistochemistry and/or imaging.

### CA4 Binding Peptides:

In certain embodiment, the invention provides a CA4 binding peptide can bind to a carbonic anhydrase IV disclosed herein (e.g., human CA4 or a homology or a variant thereof), thereby increasing permeability of the BBB (e.g., through transcytosis). In some embodiments, the increase in the permeability of the BBB is achieved by altering (e.g., increasing or decreasing) the carbonic anhydrase IV activity, such as reducing its activity.

In some embodiments, the alteration of carbonic anhydrase IV activity is achieved by a targeting peptide binding to one or more active sites of the carbonic anhydrase IV including the zinc binding site and the hydrophobic substrate binding pocket. For example, the targeting peptide can bind to the zinc binding site, the hydrophobic substrate binding pocket, or both.

Certain exemplary CA4 binding peptides, and the corresponding AAVs, which bind to mice CA4 are provided in WO 2023/168333, which is incorporated by reference in its entirety.

The AAVs are typically screened and developed using the mice CA4 to assess the BBB permeability of AAVs comprising capsids. Nonetheless, there are differences in the AAV binding pocket between murine CA4 and primate CA4. In particular, engineered msCar4-binding AAVs identified through directed evolution in mice without mechanistic insight, such as 9P31 or 9P36, do not bind to rhCA4 or huCA4 Thus, there is a need for identification of AAVs that are capable of binding to primate or human CA4. The invention recognizes that such AAVs would be able to deliver the therapeutic cargo across CNS in primates and/or humans.

While AAVs are being investigated as a method to deliver therapeutic cargo across the BBB across the CNS and in the eye, the AAVs being developed are rodent-specific PHP.B and PHP.eB are widely employed in brain disease research, their therapeutic use in non-human primates (NHPs) and clinical trials remains limited. The inadequate translation of their efficacy from rodents to primates prompts the investigation of the molecular mechanisms underlying BBB transduction and crossing by engineered AAVs.

Without being bound by the theory, the invention provides that due to protein differences in the AAV binding pocket between murine CA4 and primate CA4, previously engineered msCar4-binding AAVs identified through directed evolution in mice without mechanistic insight, such as 9P31 or 9P36, do not bind to rhCA4 or huCA4.

Thus, there is an unmet need for identification of potential membrane receptors in primates that could facilitate AAV delivery to the brain. In certain aspects, the invention provides the utilization of the pathway involving the carbonic anhydrase IV (CA4) pathway for delivering therapeutic cargo to the CNS by crossing the BBB. For example, mouse carbonic anhydrase IV (msCar4) is utilized by engineered AAV capsids 9P31 and 9P36 for extensive CNS transduction upon systemic delivery in mice. In contrast to previously identified AAV receptors, such as the murine-restricted Ly6a, CA4 and its functional mechanism are conserved across rodents and primates, including humans.

### Human or Primate CA4 Binding Peptides and AAVs:

In certain aspects, the invention provides that engineered AAVs that bind to the specific primate CA4 variant intended for the vector's ultimate application, such as rhCA4 or huCA4 are required. These AAVs are employed to traverse the BBB and transduce the CNS in primates efficiently using various non-invasive administration routes, including systemic administration.

Accordingly, in certain aspects, the invention provides methods of increasing permeability of the blood brain barrier. In some embodiments, the method comprises: providing a CA4 binding peptide (also referred to as “targeting peptide”) capable of binding to primate and/or human carbonic anhydrase 4 (CA4 or CA-IV), thereby increasing permeability of the blood brain barrier.

In certain aspects, the CA4 binding peptide is listed in Tables 1, 3, 4 and 5. In certain embodiments, the targeting peptides of the invention increase the permeability of the blood brain barrier is increased by at least 25%, 50%, 75%, 100/6, or more as compared to the absence of the targeting peptide.

In some embodiments, the CA4 binding peptide enhances the binding affinity of the viral vector or the non-viral vector to human CA4 or primate CA4. In some embodiments, the viral vector comprises an AAV vector. In some embodiments, the targeting peptide is part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. In some embodiments, the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

Accordingly, in certain aspects, the invention provides CA4 binding peptides that bind to human CA4 and/or primate CA4. Unless specified otherwise, the CA4 binding peptides referred to in this application are peptides that bind to human CA4 and/or primate CA4. In certain embodiments, the CA4 binding peptides are inserted in capsid proteins of an AAV. In certain preferred embodiments, the CA4 binding peptide is inserted between two adjacent amino acids in AA587-594 of SEQ ID NO: 1 of the AAV9 vector or functional equivalents of AA587-594 in an amino acid sequence at least 80% identical to SEQ ID NO: 1. In some embodiments, the targeting peptide is inserted between AA588-589 of SEQ ID NO: 1 of the AAV9 vector or functional equivalents of AA588-589 in an amino acid sequence at least 80% identical to SEQ ID NO: 1. In certain embodiments, the CA4 binding peptide is listed in Tables 1, 3, 4, and 5.

In certain aspects, the invention provides AAV capsid proteins that bind to primate CA4 and/or human CA4. In certain embodiments, these AAV capsid proteins comprise an insertion of CA4 binding peptides. In certain embodiments, the AAV capsid proteins or portions thereof that bind to primate and/or human CA4 are listed in Tables 1, 3, 4, and 5.

In certain aspects, the invention provides AAVs comprising AAV capsid proteins that bind to primate CA4 and/or human CA4. In certain embodiments, these AAV capsid proteins comprise an insertion of CA4 binding peptides. In certain embodiments, the AAV capsid proteins or portions thereof that bind to primate and/or human CA4 are listed in Tables 1, 3, 4, and 5.

In certain aspects, the invention provides recombinant adeno-associated virus (rAAV). In some embodiments, the rAAV comprises any of the AAV capsid protein disclosed herein. In some embodiments, the rAAV comprises an AAV capsid protein which comprises a CA4 binding peptide having a binding specificity to human CA4 and/or primate CA4, wherein the amino acid sequence of the targeting peptide is inserted between two adjacent amino acids in AA587-594, or functional equivalents thereof, of the AAV9 capsid protein. In some embodiments, the two adjacent amino acids are AA588 and AA589. In some embodiments, the rAAV has enhanced tropism for the nervous system relative to an rAAV that does not comprise the targeting peptide. In some embodiments, the rAAV is capable of transducing the nervous system with an efficiency at least 2-fold higher than an rAAV that does not comprise the targeting peptide.

In certain embodiments, the AAVs or rAAVs of the invention are employed to traverse the BBB and transduce the CNS in primates efficiently using various non-invasive administration routes, including systemic administration.

In certain embodiments, the methods of the invention enable a more translatable application of these AAVs in preclinical testing and clinical intervention for neurological diseases. In addition, in certain embodiments, the peptide modifications of these AAVs are further recruited for non-viral delivery such as for antibodies, antibody-drug conjugates, oligonucleotides, enzymes, proteins, larger synthetic molecules, exosomes, nanoparticles, and contrast agents.

In certain aspects, the invention provides carbonic anhydrase IV (CA4)-binding peptides listed in Tables 1, 3, 4, and 5. In certain aspects, the invention provides AAVs listed in Tables 1, 3, 4, and 5. These CA4 binding peptides and AAVs, listed in Tables 1, 3, 4, and 5 provide genetic access to primate central nervous system following non-invasive systemic delivery. In certain embodiments, the invention provides the 9-amino acid peptides listed in Tables 4 and 5.

In certain embodiments, the invention provides AAVs provided in Tables 4 and 5. In certain embodiments, the AAVs comprise 7-amino acid peptide insertion at 588/589 site of AAV9 and AQ or DG at position 587 to 588.

In certain aspects, the invention provides huCA4-binding AAVs Alpha 43, 45, 48, and 49. In certain embodiments, the huCA4-binding AAVs binding AAVs peptides listed in Table 1. In certain preferred embodiments, the invention provides that huCA40binding AAV peptides: DGVVHETVR; DGVVGVNIR; DGEVGLTVR; DGIVGSTIR.

In certain embodiments, the 9-amino acid peptide sequences are listed in Table 1 (below). Table 1 provides 9-amino acid peptide sequences after position 586 of individually tested AAVs variants that can bind to huCA4 or rhCA4.

TABLE 1
9-amino acid peptide sequences:

| AAV Variant | 9mer sequence after |
|---|---|
| Name | position 586 |
| Alpha_43 | DGVVHETVR (SEQ ID NO: 7) |
| Alpha_45 | DGVVGVNIR (SEQ ID NO: 8) |
| Alpha 48 | DGEVGLTVR (SEQ ID NO: 9) |
| Alpha 49 | DGIVGSTIR (SEQ ID NO: 10) |
| Omega 2 | AQTISTGIY (SEQ ID NO: 11) |
| Omega 5 | AQKWDNQQS (SEQ ID NO: 12) |
| Omega 6 | DGIFGGGGK (SEQ ID NO: 13) |
| Omega 8 | AQTKSNGIS (SEQ ID NO: 14) |
| Omega 11 | AQTVRTGIS (SEQ ID NO: 15) |

In certain embodiments, Table 2A provides the sequences for exemplary huCA4 and rhCA4 in the invention.

TABLE 2A
Human CA4 and Rhesus CA4 sequences:

| Name | Protein Sequence |
|---|---|
| huCA4 with GPI | MRMLLALLALSAARPSASAESHWCYEVQAESSNYPCLVPVKWG |
| signal peptide | GNCQKDRQSPINIVTTKAKVDKKLGRFFFSGYDKKQTWTVQNNG |
| (GPI signal | HSVMMLLENKASISGGGLPAPYQAKQLHLHWSDLPYKGSEHSLD |
| peptide in | GEHFAMEMHIVHEKEKGTSRNVKEAQDPEDEIAVLAFLVEAGTQ |
| underline) (SEQ | VNEGFQPLVEALSNIPKPEMSTTMAESSLLDLLPKEEKLRHYFRYL |
| ID NO: 1) | GSLTTPTCDEKVVWTVFREPIQLHREQILAFSQKLYYDKEQTVSM |
| | KDNVRPLQQLGQRTVIKSGAPGRPLPWALPALLGPMLACLLAGFL |
| | R |
| huCA4 with HA | MRMLLALLALSAARPSASAESHWCYEVQAESSNYPCLVPVKWG |
| tag (HA tag in | GNCQKDRQSPINIVTTKAKVDKKLGRFFFSGYDKKQTWTVQNNG |
| underline SEQ ID | HSVMMLLENKASISGGGLPAPYQAKQLHLHWSDLPYKGSEHSLD |
| NO: 2) | GEHFAMEMHIVHEKEKGTSRNVKEAQDPEDEIAVLAFLVEAGTQ |
| | VNEGFQPLVEALSNIPKPEMSTTMAESSLLDLLPKEEKLRHYFRYL |
| | GSLTTPTCDEKVVWTVFREPIQLHREQILAFSQKLYYDKEQTVSM |
| | KDNVRPLQQLGQRTVIKSGGSGYPYDVPDYA |
| rhCA4 with GPI | MRMLLALLALLALSAARPSAAAESHWCYESQANSSKDTCLVPGK |
| signal peptide | WGGNCQKDRQSPINIVTRKAKMDKKLGRFSFSGYDKKQTWTVQ |
| (GPI signal | NNGHSVMMLLGNKASISGGGLPARYQATQLHLHWSNFLDKGSE |
| peptide in | HSLDGEHFAMEVHIVHEKEKGTSRNVKEAQDPEDEIAVLAFLVEA |
| underline) (SEQ | GPQENKGFRPLVEALSNIPKPEMNTTMAESSLLDLLPKEEKLRNYF |
| ID NO: 3) | RYLGSLTTPTCDEKVVWTVFQEPIQLHREQILAFSQKLYYDKDQK |
| | LNMTNNIRPLQRPGQRVVLRSGAPGQLLPWVLPALLGPTLARLLA |
| | SFLR |
| rhCA4 with HA | MRMLLALLALLALSAARPSAAAESHWCYESQANSSKDTCLVPGK |
| tag (HA tag in | WGGNCQKDRQSPINIVTRKAKMDKKLGRFSFSGYDKKQTWTVQ |
| underline) (SEQ | NNGHSVMMLLGNKASISGGGLPARYQATQLHLHWSNFLDKGSE |
| ID NO: 4) | HSLDGEHFAMEVHIVHEKEKGTSRNVKEAQDPEDEIAVLAFLVEA |
| | GPQENKGFRPLVEALSNIPKPEMNTTMAESSLLDLLPKEEKLRNYF |
| | RYLGSLTTPTCDEKVVWTVFQEPIQLHREQILAFSQKLYYDKDQK |
| | LNMTNNIRPLQRPGQRVVLRSGGSGYPYDVPDYA |

Table 2B provides the exemplary AAV sequences for the AAV9 capsid protein and CA4 binding AAVs with the insertions of the amino acid binding peptides of the invention.

TABLE 2B
AAV capsid protein sequences:

| | |
|---|---|
| AAV9 VP1 | MAADGYLPDWLEDNLSEGIREWWALKPGAPQPKANQQHQDNARGL |
| (VP2 and | VLPGYKYLGPGNGLDKGEPVNAADAAALEHDKAYDQQLKAGDNPYL |
| VP3 in | KYNBADAEFQERLKEDTSFGGNLGRAVFQAKKRLLEPLGLVEEAAKT |
| italics) (SEQ | APGKKRPVEQSPQEPDSSAGIGKSGAQPAKKRLNFGQTGDTESVPDPQPI |
| ID NO: 5) | GEPPAAPSGVGSLTMASGGGAPVADNNEGADGVGSSSCDSQWLGDRVITT |
| | STRTWALPTYNNHLYKQISNSTSGGSSNDNAYFGYSTPWGYFD~CHFSPRD |
| | WQRLINNNWGFRPKRLNFKLFNIQVKEVTDNNGVKTIANNLTSTVQVFTDS |
| | DYQLPYVLGSAHEGCLPPFPADVFMIPQYGYLTLNDGSQAVGRSSFYCLEF |
| | PSQMLRTGNNFQFSYEFENVPFHSSYAHSQSLDRLMNPLIDQYLYYLSKTIN |
| | GSNQQTLKFSVAGPSNMAVQGRNYIPGPSYRQQRVSTTVTQNNNSEFAWP |
| | GASSWALNGRNSLMNPGPAMASHKEGEDRFFPLSGSLIFGKQGTGRDNV |
| | DADKVMITNEEEIKTTNPVATESYGQVATNHQSAQAQAQTGWVQNQGILP |
| | GMVWQDRDVYLQGPIWAKIPHTDNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRN |
| CA4- | MAADGYLPDWLEDNLSEGIREWWALKPGAPQPKANQQHQDNARGL |
| binding | VLPGYKYLGPGNGLDKGEPVNAADAAALEHDKAYDQQLKAGDNPYL |
| AAVs (9- | KYNBADAEFQERLKEDTSFGGNLGRAVFQAKKRLLEPLGLVEEAAKT |
| amino | APGKKRPVEQSPQEPDSSAGIGKSGAQPAKKRLNFGQTGDTESVPDPQ |
| acid | PIGEPPAAPSGVGSLTMASGGGAPVADNNEGADGVGSSSGNWHCDSQ |
| peptide | WLGDRVITTSTRTWALPTYNNHLYKQISNSTSGGSSNDNAYFGYSTPW |
| XXXXXXX | GYFDFNRFHCHFSPRDWQRLINNNWGFRPKRLNFKLFNIQVKEVTDN |
| XX) ((SEQ | NGVKTIANNLTSTVQVFTDSDYQLPYVLGSAHEGCLPPFPADVFMIPQ |
| ID NO: 6) | YGYLTLNDGSQAVGRSSFYCLEYFPSQMLRTGNNFQFSYEFENVPFHS |
| | SYAHSQSLDRLMNPLIDQYLYYLSKTINGSGQNQQTLKFSVAGPSNMA |
| | VQGRNYIPGPSYRQQRVSTTVTQNNNSEFAWPGASSWALNGRNSLMN |
| | PGPAMASHKEGEDRFFPLSGSLIFGKQGTGRDNVDADKVMITNEEEIK |
| | TTNPVATESYGQVATNHQSXXXXXXXXXAQAQTGWVQNQGILPGM |
| | VWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYT |
| | SNYYKSNNVEFAVNTEGVYSEPRPIGTRYLTRNL |

In certain embodiments, the CA4 binding 9-amino acid peptides are provided in Table 3. Table 3 provides the exemplary 9-amino acid CA4 binding peptides along with the pertinent AAV variants.

TABLE 3
CA4 binding 9-amino acid peptide sequences and AAVs:

| Variant | 9mer sequence | AAV Variant | 9mer sequence |
|---|---|---|---|
| Name | after position | Name | after position |
| Alpha 41 | AQTAMEFDH (SEQ ID NO: | Omega 40 | AQTYNENSL (SEQ ID NO: 30) |
| | 16) | | |
| Alpha 59 | AQVPEQHIK (SEQ ID NO: | Omega 41 | AQKNLRWGH (SEQ ID NO: |
| | 17) | | 31) |
| Alpha 60 | AQWESHIR (SEQ ID NO: 18) | Omega 42 | AQLRKANRP (SEQ ID NO: 32) |
| Alpha 61 | AQTYPNYNS (SEQ ID NO: | Omega 43 | AQGTYCAGQ ((SEQ ID NO: |
| | 19) | | 33) |
| Alpha 62 | DGIVHLAVR (SEQ ID NO: | Omega 44 | AQSAIRMKC (SEQ ID NO: 34) |
| | 20) | | |
| Alpha 63 | DGLLEYTFR (SEQ ID NO: | Omega 45 | AQIRWVRGV (SEQ ID NO: 35) |
| | 21) | | |
| Alpha 64 | AQIGDYFGV (SEQ ID NO: | Omega 46 | AQAVMKRIK (SEQ ID NO: 36) |
| | 22) | | |
| Omega 33 | AQVDVWGRA (SEQ ID NO: | Omega 47 | AQIIIKMLR (SEQ ID NO: 37) |
| | 23) | | |
| Omega 34 | AQMDGWGRT (SEQ ID NO: | Omega 48 | AQHRTCVAF (SEQ ID NO: 38) |
| | 24) | | |
| Omega 35 | AQTRFIEPY (SEQ ID NO: 25) | Omega 49 | AQSGGCAGC (SEQ ID NO: 39) |
| Omega 36 | AQTSTLVFH (SEQ ID NO: | Omega 50 | AQSFVRFSW (SEQ ID NO: 40) |
| | 26) | | |
| Omega 37 | AQFVMYAYD (SEQ ID NO: | Omega 51 | AQSVHFRCG (SEQ ID NO: 41) |
| | 27) | | |
| Omega 38 | AQKFREGDI (SEQ ID NO: | Omega 52 | AQIMSCTMF (SEQ ID NO: 42) |
| | 28) | | |
| Omega 39 | AQKWDLTDR (SEQ ID NO: | | |
| | 29) | | |

In certain embodiments, the 9-mer amino acid peptides that bind to human CA4 (huCA4) are provided in Table 4. In certain preferred embodiments, the 9-mer amino acid peptides in Table 4 are after position 586 of AAV9 capsid protein or a functional equivalent thereof.

TABLE 4
Human CA-IV binding 9-mer amino acid peptides

| SEQ | |
|---|---|
| ID NO | 9-mer |
| 43 | AQDKAYTNS |
| 44 | AQVQRANPI |
| 45 | AQENPSRGT |
| 46 | AQSTRLNSV |
| 47 | AQYGAGSAM |
| 48 | AQRVDPSGL |
| 49 | AQARLPSSP |
| 50 | AQTNIPPRT |
| 51 | AQTRNLDVA |
| 52 | AQLSPHAGA |
| 53 | AQLSNLTTN |
| 54 | AQADPARYM |
| 55 | AQSSSATRS |
| 56 | AQNASTVSR |
| 57 | AQTAPLRGL |
| 58 | AQNSSGDRS |
| 59 | AQTSHNANR |
| 60 | AQSTNANPG |
| 61 | AQNTSTLRP |
| 62 | AQVLSLSNP |
| 63 | AQVAPFSSG |
| 64 | AQLMKPSSV |
| 65 | AQGVIRVDS |
| 66 | AQTTSSTSY |
| 67 | AQVQRGTLT |
| 68 | AQNALVNQF |
| 69 | AQPTTVTPR |
| 70 | AQGLPWTSE |
| 71 | AQPPIANPY |
| 72 | AQTTRELLK |
| 73 | AQRYPGEGP |
| 74 | AQLATATHS |
| 75 | AQRVPSNEL |
| 76 | AQLTGHHNP |
| 77 | AQSHPLKVP |
| 78 | AQTDFKHMV |
| 79 | AQWMALTAA |
| 80 | AQVNKTISM |
| 81 | AQSIRPVPT |
| 82 | AQKTNASLL |
| 83 | AQGSPGPAM |
| 84 | AQFNKLPSS |
| 85 | AQGSLRRGM |
| 86 | AQRLRDLPN |
| 87 | AQSGPKAPG |
| 88 | AQALIGSTA |
| 89 | AQGRTTTGT |
| 90 | AQKLESIMG |
| 91 | AQSTINSPS |
| 92 | AQSFAAAQP |
| 93 | AQIPRPNVA |
| 94 | AQYQNGPIQ |
| 95 | AQARIVSGA |
| 96 | AQSKVISPP |
| 97 | AQQAGLMAA |
| 98 | AQSSPPGKD |
| 99 | AQTMSMPKV |
| 100 | AQTVAPPRH |
| 101 | AQRTNDKVM |
| 102 | AQVVRPYTV |
| 103 | AQTLGHSTM |
| 104 | AQMPQYQPG |
| 105 | AQSPPSHKH |
| 106 | AQAGKAFEF |
| 107 | AQDLRGLSH |
| 108 | AQAPSYTTL |
| 109 | AQSQSHTNT |
| 110 | AQVFSNSVP |
| 111 | AQLQRSGVH |
| 112 | AQAKSAQTT |
| 113 | AQLSTVRDL |
| 114 | AQRFEVGSQ |
| 115 | AQLLADTRP |
| 116 | AQDLHTRGG |
| 117 | AQPPTHRTA |
| 118 | AQRETPGVN |
| 119 | AQHLPVSQA |
| 120 | AQLKLGSLS |
| 121 | AQHNGWTAD |
| 122 | AQGQSVVNT |
| 123 | AQVNDVSHR |
| 124 | AQEHTSELS |
| 125 | AQAAVVHRG |
| 126 | AQKIAGTAD |
| 127 | AQGALRESM |
| 128 | AQRPPELNR |
| 129 | AQSVVTHLA |
| 130 | AQSAKPGVF |
| 131 | AQPLRLSTE |
| 132 | AQPHVPGAG |
| 133 | AQSSSVPMG |
| 134 | AQTAQMSAP |
| 135 | AQTRGDPTS |
| 136 | AQMPRITTS |
| 137 | AQIELRGNH |
| 138 | AQEYGIDRL |
| 139 | AQLAAPVKT |
| 140 | AQSRPQIGS |
| 141 | AQNMSLSNY |
| 142 | AQIPHREPT |
| 143 | AQISSVRVS |
| 144 | AQLEYKHAL |
| 145 | AQYMKAVAT |
| 146 | AQLPPNHRG |
| 147 | AQHTNNSYA |
| 148 | AQSYIKTSA |
| 149 | AQVGKNEIY |
| 150 | AQRVSNDLM |
| 151 | AQRDHRNTD |
| 152 | AQHPEIRLT |
| 153 | AQYGVLNHS |
| 154 | AQNTPSSHA |
| 155 | AQLYESYKP |
| 156 | AQNVAGSVF |
| 157 | AQAAQRTAL |
| 158 | AQHNHFETV |
| 159 | AQHVAKHEP |
| 160 | AQGKSANLS |
| 161 | AQATSREAM |
| 162 | AQVPVHLPT |
| 163 | AQFGQVTTN |
| 164 | AQHPSGYPT |
| 165 | AQSFTLPNS |
| 166 | AQNDVTFAG |
| 167 | AQELHKSLP |
| 168 | AQDGSNILL |
| 169 | AQLQGNPAA |
| 170 | AQKGVTDNT |
| 171 | AQVVRTVAD |
| 172 | AQGFKTKEI |
| 173 | AQKIPTWDD |
| 174 | AQFTSLVTP |
| 175 | AQPPKYGTH |
| 176 | AQTVKRVEL |
| 177 | AQGARMVRE |
| 178 | AQPLARDVT |
| 179 | AQFLSGTSN |
| 180 | AQVNLTLPP |
| 181 | AQTTPLPAV |
| 182 | AQLGVKILL |
| 183 | AQGRVNNGG |
| 184 | AQVGLGPTN |
| 185 | AQNTQAYGA |
| 186 | AQEHTSNSS |
| 187 | AQGSKFEIS |
| 188 | AQTLRELGT |
| 189 | AQPIRASMV |
| 190 | AQRGSSGVS |
| 191 | AQDRHMSMA |
| 192 | AQVRISGVG |
| 193 | AQTRQASLS |
| 194 | AQSCTSWVQ |
| 195 | AQPQTSAYL |
| 196 | AQNHLIQKA |
| 197 | AQLPQRIAT |
| 198 | AQVAPGNSA |
| 199 | AQPNVMQMP |
| 200 | AQFSRELPD |
| 201 | AQLGSKPRR |
| 202 | AQNRASNTL |
| 203 | AQVPPVGRL |
| 204 | AQSQYSTGV |
| 205 | AQGFPPVAP |
| 206 | AQTHASTAE |
| 207 | AQSPNTRDP |
| 208 | AQVSRAPLP |
| 209 | AQIASDPNA |
| 210 | AQQGTNHYA |
| 211 | AQIPNLREG |
| 212 | AQMQGMKFQ |
| 213 | AQNATRANP |
| 214 | AQLPHSSTH |
| 215 | AQRTPTMMM |
| 216 | AQKSPGPFV |
| 217 | AQNQGSPNH |
| 218 | AQAPMGYRG |
| 219 | AQAMSNFSP |
| 220 | AQTVCLGLR |
| 221 | AQNDLQRNA |
| 222 | AQGAGSSIS |
| 223 | AQKHGSSSM |
| 224 | AQNLYTPHP |
| 225 | AQRNKPGDD |
| 226 | AQRTPPYSN |
| 227 | AQVGANHFS |
| 228 | AQGARLTTT |
| 229 | AQTVVLGIS |
| 230 | AQIIRRVLL |
| 231 | AQQGDRKST |
| 232 | AQRVGFKTK |
| 233 | AQEHTLNSS |
| 234 | AQHTSELQS |
| 235 | AQSTGWVQN |
| 236 | AQGLSGLLS |
| 237 | AQECGGGGG |
| 238 | AQRAAVCIL |
| 239 | AQCVLKCGE |
| 240 | AQSTRLNST |
| 241 | AQVRIRRLM |
| 242 | AQWKFVATE |
| 243 | AQGCMLRKE |
| 244 | AQGVVAMAR |
| 245 | AQKMWPTHD |
| 246 | AQAVVRTDG |
| 247 | AQMNPSSSR |
| 248 | AQSRLGSKP |
| 249 | AQRVDQTHQ |
| 250 | AQARTKLYL |
| 251 | AQLPVCTPA |
| 252 | AQTTALQYS |
| 253 | AQGKNACFE |
| 254 | AQKARVSFA |
| 255 | AQPTLQFNE |
| 256 | AQQNRLRFK |
| 257 | AQVRYVCLD |
| 258 | AQQSLMDLV |
| 259 | AQNPERVVA |
| 260 | AQSFVRFSW |
| 261 | AQLRVGLSV |
| 262 | AQNQGDRKS |
| 263 | AQDKPGQGK |
| 264 | AQSTRLNCW |
| 265 | AQSLKCDIR |
| 266 | AQMSSTCMA |
| 267 | AQGNSLRYS |
| 268 | AQSFRKCDV |
| 269 | AQTSLRVNC |
| 270 | AQMNTRTIK |
| 271 | AQMMECRQA |
| 272 | AQGICYGRY |
| 273 | AQHTGHKTN |
| 274 | AQTMLRYGD |
| 275 | AQSVTKNGV |
| 276 | AQQLCDPDY |
| 277 | AQTINSKTA |
| 278 | AQQKCRYGD |
| 279 | AQTVIKMMS |
| 280 | AQNTGSIRL |
| 281 | AQSTVGFKT |
| 282 | AQSMVRING |
| 283 | AQTLIRASL |
| 284 | AQIGVIHCA |
| 285 | AQPTSSGVE |
| 286 | AQGEERRIA |
| 287 | AQQVTYQKN |
| 288 | AQEHTSESS |
| 289 | AQWNYMNRA |
| 290 | AQVRESAMN |
| 291 | AQLRCSVGE |
| 292 | AQDKPGQAK |
| 293 | AQRWLRVGL |
| 294 | AQYDPLTVK |
| 295 | AQRGTSFRS |
| 296 | AQCLSSVVC |
| 297 | AQSRTQRVM |
| 298 | AQAVVREGY |
| 299 | AQVFTSKLC |
| 300 | AQMRDKCTS |
| 301 | AQGMRTKYN |
| 302 | AQLLKEACW |
| 303 | AQVVLNACK |
| 304 | AQQSSYKCQ |
| 305 | AQTQSMQYT |
| 306 | AQTKEIGRA |
| 307 | AQIINCAET |
| 308 | AQPLNVKEQ |
| 309 | AQVCTMPLY |
| 310 | AQLTHTVNL |
| 311 | AQPESETVT |
| 312 | AQNCQKHAS |
| 313 | AQLRTFSWT |
| 314 | AQRECSSAD |
| 315 | AQYCQGLLT |
| 316 | AQLKAYRCA |
| 317 | AQVNERRSR |
| 318 | AQNRRSQTG |
| 319 | AQKMQIIIS |
| 320 | AQINSCEEH |
| 321 | AQVARTVCG |
| 322 | AQNDEYMCL |
| 323 | AQICIPSQS |
| 324 | AQEHRLNSS |
| 325 | AQSWKGDVD |
| 326 | AQQASCAHV |
| 327 | AQGGARGYT |
| 328 | AQAGKLFNR |
| 329 | AQEPNKHCV |
| 330 | AQCNADIVS |
| 331 | AQDRGSFVS |
| 332 | AQSTKMQMS |
| 333 | AQGSKPRRS |
| 334 | AQRAFVELS |
| 335 | AQNSSVNRC |
| 336 | AQRNPIGVL |
| 337 | AQILVLLIR |
| 338 | AQMDMTMRN |
| 339 | AQAISWKSG |
| 340 | AQLSSPCSV |
| 341 | AQPMHNMRW |
| 342 | AQGKIKAFL |
| 343 | AQKLSNAMC |
| 344 | AQKPQLLCA |
| 345 | AQRNEADSC |
| 346 | AQPRNIRLS |
| 347 | AQLASHCNS |
| 348 | AQGTGLCNA |
| 349 | AQNRQCSVM |
| 350 | AQSSSHLRS |
| 351 | AQIYRGGVE |
| 352 | AQMETQCKT |
| 353 | AQAYDPNKY |
| 354 | AQLTSFGAA |
| 355 | AQGIDDLCT |
| 356 | AQGPYLCSD |
| 357 | AQMTRPDPC |
| 358 | AQKMNPRHM |
| 359 | AQEIRHPNS |
| 360 | AQVSTCIDI |
| 361 | AQKHENDAC |
| 362 | AQMPFIAKS |
| 363 | AQLFGLTAC |
| 364 | AQFMPDFQS |
| 365 | AQALPMMRL |
| 366 | AQGKCFEHM |
| 367 | AQSCNSSCL |
| 368 | AQADSSCNM |
| 369 | AQNHVDAVR |
| 370 | AQVARTSGN |
| 371 | AQIPSREHK |
| 372 | AQQHSLRFK |
| 373 | AQCNITQRA |
| 374 | AQSVYCGAV |
| 375 | AQCRTSSGD |
| 376 | AQVPTKHSH |
| 377 | AQGVIDARC |
| 378 | AQSQTMMTR |
| 379 | AQCTGDLRE |
| 380 | AQLGHTSCS |
| 381 | AQRRSIRAF |
| 382 | AQFLKSARS |
| 383 | AQISRVRLR |
| 384 | AQAGCNNLD |
| 385 | AQKHVSNED |
| 386 | AQFPNLVCP |
| 387 | AQECYQMRS |
| 388 | AQGHDRNPR |
| 389 | AQIGCYESK |
| 390 | AQHSVASDC |
| 391 | AQIKVVGLS |
| 392 | AQEHTSDSS |
| 393 | AQFGLEVAV |
| 394 | AQRVVVCTG |
| 395 | AQKHKMCQG |
| 396 | AQAWWQEFG |
| 397 | AQLCQPTRN |
| 398 | AQLVAGREL |
| 399 | AQCSQVKLT |
| 400 | AQTRIVRNL |
| 401 | AQQGYCNEY |
| 402 | AQCGEMWGW |
| 403 | AQEHTSALQ |
| 404 | AQRDMTYPW |
| 405 | AQSPVRCGL |
| 406 | AQEQRLNSS |
| 407 | AQVSVTVVA |
| 408 | AQMGHYSVH |
| 409 | AQLMASNAA |
| 410 | AQVVESHIR |
| 411 | AQFPEQFLV |
| 412 | AQSEVSAFI |
| 413 | AQWGQTVPG |
| 414 | AQHGGGSRY |
| 415 | AQVICSALT |
| 416 | AQIICSGVK |
| 417 | AQLICSTIM |
| 418 | AQIICSEIV |
| 419 | AQTMVGPGV |
| 420 | AQVICDSRN |
| 421 | AQSELQAFV |
| 422 | AQLVCSTLR |
| 423 | AQTIMSHNV |
| 424 | AQTATGGKT |
| 425 | AQIIESHVR |
| 426 | AQFVEQFIR |
| 427 | AQVVCANIV |
| 428 | AQLICSTLV |
| 429 | AQUICSDIR |
| 430 | AQEPSRVGN |
| 431 | AQLICSTLI |
| 432 | AQVPEQHIK |
| 433 | AQTVRTGIS |
| 434 | AQSVWTGSV |
| 435 | AQIICTTVR |
| 436 | AQVIEGHFK |
| 437 | AQTILQLGE |
| 438 | AQTISTGIY |
| 439 | AQMESQYAW |
| 440 | AQLVCSNLR |
| 441 | AQVICSNLY |
| 442 | AQTYPKYAT |
| 443 | AQIVCAHRI |
| 444 | AQYKERIRD |
| 445 | AQKPGRPII |
| 446 | AQVICSSML |
| 447 | AQVLCSSIK |
| 448 | AQTYPNYNS |
| 449 | AQVVCSTVR |
| 450 | AQVVCSHTM |
| 451 | AQSNSIRNT |
| 452 | AQTELTAFP |
| 453 | AQDNRTPGR |
| 454 | AQVICSALS |
| 455 | AQIMDNMRW |
| 456 | AQLICANMV |
| 457 | AQRVPMNRG |
| 458 | AQTQSPLPM |
| 459 | AQVVCSGMR |
| 460 | AQLICTTIK |
| 461 | AQRDSMSTR |
| 462 | AQSMAQNNK |
| 463 | AQNMSLAFK |
| 464 | AQSTSTHNY |
| 465 | AQHSRNVVQ |
| 466 | AQLICSGLN |
| 467 | AQIRNIRNA |
| 468 | AQLLCTTIR |
| 469 | AQVLCSGLI |
| 470 | AQAMTALGA |
| 471 | AQQNSMSFK |
| 472 | AQLKPKAYG |
| 473 | AQIGDYFGV |
| 474 | AQAICSTIW |
| 475 | AQRMSSNYN |
| 476 | AQSVISLGE |
| 477 | AQGGPMKKI |
| 478 | AQNPVARSY |
| 479 | AQIHSNIRS |
| 480 | AQNVVGLGK |
| 481 | AQTVSYQVN |
| 482 | AQYPEMHIR |
| 483 | AQFGKTWHV |
| 484 | AQAYDPNAR |
| 485 | AQFVNMRSG |
| 486 | AQTMLKAGE |
| 487 | AQKAGSSFK |
| 488 | AQRALERKG |
| 489 | AQVLCSSLK |
| 490 | AQSSCYGVW |
| 491 | AQFSAGWKA |
| 492 | AQSVIKSGE |
| 493 | AQLLEPTRM |
| 494 | AQSTLNQRS |
| 495 | AQSHTSTRL |
| 496 | AQKSGGCAG |
| 497 | AQTARSGIY |
| 498 | AQSMTAKNL |
| 499 | AQVVEGHVK |
| 500 | AQDNRATRS |
| 501 | AQSVSAQRS |
| 502 | AQSPGLRTS |
| 503 | AQLICSSVV |
| 504 | AQESSYSYK |
| 505 | AQLVCTTQR |
| 506 | AQQWELTPM |
| 507 | AQVICSELT |
| 508 | AQRPNRPLN |
| 509 | AQDARLLRT |
| 510 | AQNSVSGRF |
| 511 | AQTVQHQSS |
| 512 | AQNEVVWRV |
| 513 | AQHPLLTFG |
| 514 | AQGKGLSTI |
| 515 | AQVQFNRDF |
| 516 | AQSVLHQIH |
| 517 | AQLICTTLY |
| 518 | AQHPVGLGM |
| 519 | AQQNMRYQG |
| 520 | AQVREKDKN |
| 521 | AQNIRVQID |
| 522 | AQVISYQIN |
| 523 | AQTTTSQRE |
| 524 | AQQQRPSLT |
| 525 | AQETALRRA |
| 526 | AQDWAVCAN |
| 527 | AQQDPVRKT |
| 528 | AQVICSKLV |
| 529 | AQDSSPNKP |
| 530 | AQRPRNPTG |
| 531 | AQMLCTTIR |
| 532 | AQSRYNSKP |
| 533 | AQQDKPNRT |
| 534 | AQTGTKVYI |
| 535 | AQPMPRSPG |
| 536 | AQTFKVQRD |
| 537 | AQIVCSQLV |
| 538 | AQTLRNGIS |
| 539 | AQFINGPEH |
| 540 | AQNAMRQIT |
| 541 | AQRGLQNNK |
| 542 | AQSVTFQLS |
| 543 | AQLSGGLRT |
| 544 | AQRDKIGHG |
| 545 | AQRMLKHVN |
| 546 | AQTAWEYDN |
| 547 | AQKPIGMGN |
| 548 | AQIVCANNR |
| 549 | AQLAERSRM |
| 550 | AQGGLPKYG |
| 551 | AQFIVSHNV |
| 552 | AQRLERSVL |
| 553 | AQRSDIRRP |
| 554 | AQSPVKTGL |
| 555 | AQVREFKKD |
| 556 | AQMLKMLVA |
| 557 | AQTPLALGE |
| 558 | AQRSTVTNM |
| 559 | AQVRLLAKP |
| 560 | AQRAELTKY |
| 561 | AQGRLKENM |
| 562 | AQNITKLGA |
| 563 | AQHTNARIL |
| 564 | AQSASKSWS |
| 565 | AQAFDVNGR |
| 566 | AQVICAYLM |
| 567 | AQTTTNQIK |
| 568 | AQSLVRMGE |
| 569 | AQVSVRPER |
| 570 | AQRKESCSR |
| 571 | AQVQEVANR |
| 572 | AQTTNTGIS |
| 573 | AQVGEVRLK |
| 574 | AQGQDPVKN |
| 575 | AQGGGLTYV |
| 576 | AQVLCTYNK |
| 577 | AQNHIRPGA |
| 578 | AQRFQVNSR |
| 579 | AQYTTQQTS |
| 580 | AQHIGLQID |
| 581 | AQGSRSSFN |
| 582 | AQRDGGRSV |
| 583 | AQGVAKLGL |
| 584 | AQNQTPIRS |
| 585 | AQVKRMINL |
| 586 | AQNTLSQMS |
| 587 | AQLICSTVV |
| 588 | AQVMRMQDK |
| 589 | AQSPFKGGV |
| 590 | AQHVSKMHG |
| 591 | AQLSQGLRH |
| 592 | AQSSRQGPA |
| 593 | AQNMLGLGQ |
| 594 | AQTICTTIR |
| 595 | AQFMTRQSM |
| 596 | AQLVCSDLV |
| 597 | AQTMALGIS |
| 598 | AQMYGLMEE |
| 599 | AQLTTHQVS |
| 600 | AQWCTRPCG |
| 601 | AQSVATPRS |
| 602 | AQALTVRSM |
| 603 | AQSSRTTYA |
| 604 | AQPYDPNRN |
| 605 | AQNSRLSNT |
| 606 | AQVSNHSLR |
| 607 | AQEPTLKRT |
| 608 | AQAVTPRNI |
| 609 | AQQSPVKMD |
| 610 | AQRGHVGNG |
| 611 | AQHRLSVPS |
| 612 | AQYGKWVQA |
| 613 | AQLKAHGYS |
| 614 | AQIGERPSL |
| 615 | AQTVLKQIE |
| 616 | AQIATIKSK |
| 617 | AQVVCSTIR |
| 618 | AQSPIGIHG |
| 619 | AQQAPIRLG |
| 620 | AQIICSGYK |
| 621 | AQTRINGIY |
| 622 | AQSMTGIGM |
| 623 | AQRVTGMGH |
| 624 | AQLHTRTST |
| 625 | AQLNKRIVY |
| 626 | AQTTTLQVD |
| 627 | AQNKLDRRN |
| 628 | AQTVTMQYA |
| 629 | AQSMTPHPG |
| 630 | AQYRSPNVK |
| 631 | AQLICTSLM |
| 632 | AQRSHTTLT |
| 633 | AQNVLHQIS |
| 634 | AQSKKFGQS |
| 635 | AQTVYSGIS |
| 636 | AQLTTHQFS |
| 637 | AQVVCSNVI |
| 638 | AQIPQIRSS |
| 639 | AQLICSSVR |
| 640 | AQTVTRQTT |
| 641 | AQVLDNIRW |
| 642 | AQYVTMQVQ |
| 643 | AQENDIRRY |
| 644 | AQTVLTQHS |
| 645 | AQTWSTGAS |
| 646 | AQLICTTVP |
| 647 | AQSQGDVRR |
| 648 | AQMNERWHV |
| 649 | AQVVTASRS |
| 650 | AQVICMGVI |
| 651 | AQSPVKLSQ |
| 652 | AQLPSGVRH |
| 653 | AQDPVKLGR |
| 654 | AQQKPKAEF |
| 655 | AQTTLSQCS |
| 656 | AQGRPPPSS |
| 657 | AQSMGRMAS |
| 658 | AQGSNGMRF |
| 659 | AQGKSLVFT |
| 660 | AQNTVMQTS |
| 661 | AQGPLREGM |
| 662 | AQMPTGMGL |
| 663 | AQGVVRQTS |
| 664 | AQMINTHKL |
| 665 | AQSIGVYGL |
| 666 | AQRGKIEHH |
| 667 | AQNILRMGN |
| 668 | AQVKNRLTN |
| 669 | AQQQSPVYM |
| 670 | AQYVVSSKQ |
| 671 | AQMITRQID |
| 672 | AQRTMTSNR |
| 673 | AQGPIQAGG |
| 674 | AQSVVKLST |
| 675 | AQRMTQVGQ |
| 676 | AQTPIRESY |
| 677 | AQAMVKLGG |
| 678 | AQKGRFVPE |
| 679 | AQQPGGMVR |
| 680 | AQGGHVGNG |
| 681 | AQVVCSSLL |
| 682 | AQGNYTGRS |
| 683 | AQLAMTAHK |
| 684 | AQLSPKAGY |
| 685 | AQAGMRPNI |
| 686 | AQFKMVSSS |
| 687 | AQVICTTNR |
| 688 | AQKYLPKDC |
| 689 | AQNVGVRNV |
| 690 | AQLVRSMER |
| 691 | AQMMLKMGT |
| 692 | AQLHKPAER |
| 693 | AQNQTPVKW |
| 694 | AQIRNVNST |
| 695 | AQRLTDNPR |
| 696 | AQISSSVRL |
| 697 | AQWRSNSLS |
| 698 | AQRQSPVHS |
| 699 | AQTVMSQVS |
| 700 | AQSPKGIYF |
| 701 | AQTLNKQIT |
| 702 | AQKNNLRSD |
| 703 | AQRVIDLNR |
| 704 | AQTNKKENS |
| 705 | AQRVSTGIS |
| 706 | AQTVVRQMG |
| 707 | AQEGLKGRA |
| 708 | AQHMKDRVN |
| 709 | AQSPIRGGE |
| 710 | AQSLLSQLS |
| 711 | AQHFKPNNL |
| 712 | AQSMVAGGG |
| 713 | AQGPVGMTG |
| 714 | AQGVVREGA |
| 688 | AQKYLPKDC |
| 689 | AQNVGVRNV |
| 690 | AQLVRSMER |
| 691 | AQMMLKMGT |
| 692 | AQLHKPAER |
| 693 | AQNQTPVKW |
| 694 | AQIRNVNST |
| 695 | AQRLTDNPR |
| 696 | AQISSSVRL |
| 697 | AQWRSNSLS |
| 698 | AQRQSPVHS |
| 699 | AQTVMSQVS |
| 700 | AQSPKGIYF |
| 701 | AQTLNKQIT |
| 702 | AQKNNLRSD |
| 703 | AQRVIDLNR |
| 704 | AQTNKKENS |
| 705 | AQRVSTGIS |
| 706 | AQTVVRQMG |
| 707 | AQEGLKGRA |
| 708 | AQHMKDRVN |
| 709 | AQSPIRGGE |
| 710 | AQSLLSQLS |
| 711 | AQHFKPNNL |
| 712 | AQSMVAGGG |
| 713 | AQGPVGMTG |
| 714 | AQGVVREGA |
| 715 | AQPSPAGRT |
| 716 | AQEPKDEWR |
| 717 | AQHLTNRTH |
| 718 | AQRGSSWGA |
| 719 | AQDNRLHYP |
| 720 | AQNIGSYSL |
| 721 | AQSPIPAGA |
| 722 | AQYINGPSM |
| 723 | AQRPANLNR |
| 724 | AQSAGYSKG |
| 725 | AQTIWSGGP |
| 726 | AQSHTNSRA |
| 727 | AQTSYTGIS |
| 728 | AQTLYKYYP |
| 729 | AQYGAAWHV |
| 730 | AQHMIKLGE |
| 731 | AQTFQMQIA |
| 732 | AQLVFGRDM |
| 733 | AQTLNTHNT |
| 734 | AQSMIKLGL |
| 735 | AQDRLKMVS |
| 736 | AQTVWEGGD |
| 737 | AQISKPNRT |
| 738 | AQIISYTRA |
| 739 | AQMHTSHNT |
| 740 | AQIIATHNN |
| 741 | AQRLKGEWT |
| 742 | AQIKLIVKS |
| 743 | AQQVVKMGL |
| 744 | AQHMPSPLR |
| 745 | AQTIIRFGQ |
| 746 | AQSMVPRNS |
| 747 | AQNPVKMGA |
| 748 | AQSMVGGNP |
| 749 | AQAWKDSDL |
| 750 | AQVVSGPRT |
| 751 | AQTFVKQIT |
| 752 | AQTVVKMGT |
| 753 | AQIRPDYFY |
| 754 | AQREIRTSS |
| 755 | AQFQSPVSS |
| 756 | AQSVVALGS |
| 757 | AQTMAQNKQ |
| 758 | AQYKTSKNA |
| 759 | AQGSSLLFK |
| 760 | AQPFPRTNP |
| 761 | AQRESVRLR |
| 762 | AQILTKLYT |
| 763 | AQIQYKTVG |
| 764 | AQFGRMAVS |
| 765 | AQTDPHSNF |
| 766 | AQDFRTARQ |
| 767 | AQKVTGMGI |
| 768 | AQTRLYKSP |
| 769 | AQRSARPPS |
| 770 | AQLVCTYAI |
| 771 | AQSHKLGVH |
| 772 | AQMRYGGGS |
| 773 | AQRDPDRNL |
| 774 | AQGGYQLKG |
| 775 | AQSHSKTAF |
| 776 | AQVGGGMSN |
| 777 | AQSKRLSQS |
| 778 | AQNPVGGRG |
| 779 | AQSGRPTST |
| 780 | AQMSAQPRL |
| 781 | AQSRNSVYN |
| 782 | AQLICSSLK |
| 783 | AQTSVKLKV |
| 784 | AQTPMSRHV |
| 785 | AQRTGYGTG |
| 786 | AQVICSTGM |
| 787 | AQANSHRSP |
| 788 | AQRPEGPHK |
| 789 | AQWINSGPS |
| 790 | AQNLYTNRS |
| 791 | AQCASGKYG |
| 792 | AQTVLRQGE |
| 793 | AQRDAKLMD |
| 794 | AQFHMSLKD |
| 795 | AQIICSGRY |
| 796 | AQVIVHSPA |
| 797 | AQALTQPSR |
| 798 | AQTPVKYGS |
| 799 | AQRSVGSVY |
| 800 | AQLKPKSTA |
| 801 | AQTMLKLGD |
| 802 | AQFVAAKTQ |
| 803 | AQISPLFTK |
| 804 | AQTTRSSMC |
| 805 | AQTVCSNLK |
| 806 | AQSPLRAGA |
| 807 | AQQIGGRNL |
| 808 | AQSVVYQLT |
| 809 | AQVVSESQG |
| 810 | AQKYPSVSS |
| 811 | AQKTNYSNM |
| 812 | AQISPGRYL |
| 813 | AQTMLKGGL |
| 814 | AQVVCPKNE |
| 815 | AQAMARGRV |
| 816 | AQTQWSGIS |
| 817 | AQVLRSQID |
| 818 | AQSRILPVS |
| 819 | AQGHTGIHS |
| 820 | AQPMPARTH |
| 821 | AQMRQAAKD |
| 822 | AQQNPVRAL |
| 823 | AQRVPQAGR |
| 824 | AQSPTRYGE |
| 825 | AQMNDIRRH |
| 826 | AQNSSGRSG |
| 827 | AQKISSHNI |
| 828 | AQSMPSPNR |
| 829 | AQCSNRMPK |
| 830 | AQGSSLRSM |
| 831 | AQIGEYRLK |
| 832 | AQSMIKMST |
| 833 | AQNTLSQLR |
| 834 | AQSNPKRAW |
| 835 | AQTFPNSVR |
| 836 | AQTMHGTNS |
| 837 | AQIWRKAIA |
| 838 | AQTVGSRSA |
| 839 | AQQKMTYYE |
| 840 | AQVMTKLGP |
| 841 | AQAKHDELV |
| 842 | AQRVVKQGE |
| 843 | AQKHSLAYT |
| 844 | AQLNDATRY |
| 845 | AQISGVRFA |
| 846 | AQVICANQR |
| 847 | AQVLSVKDR |
| 848 | AQFLKEYNT |
| 849 | AQKGSFVSA |
| 850 | AQSFDPNGS |
| 851 | AQHSVRRDP |
| 852 | AQSVTQQVT |
| 853 | AQSPIKLSH |
| 854 | AQTVVKHGL |
| 855 | AQKFVRSPV |
| 856 | AQYSPVVEK |
| 857 | AQMRGNLNV |
| 858 | AQSMVPKLS |
| 859 | AQSAQRASA |
| 860 | AQFVDPNQR |
| 861 | AQTALSQHS |
| 862 | AQNAFNKQR |
| 863 | AQESPGRIT |
| 864 | AQLRIPGPR |
| 865 | AQRSSLSSL |
| 866 | AQNAANSYK |
| 867 | AQSRAEWKD |
| 868 | AQTQGKLHP |
| 869 | AQMQTIGSY |
| 870 | AQAPGLRYN |
| 871 | AQRPSDRMS |
| 872 | AQMTRTSAS |
| 873 | AQTILKLGS |
| 874 | AQVVEGHWM |
| 875 | AQEVVMRTS |
| 876 | AQVRPSQIR |
| 877 | AQWVVSLDR |
| 878 | AQLNRENRF |
| 879 | AQIARIAVQ |
| 880 | AQFRTGKEL |
| 881 | AQDVVRQGY |
| 882 | AQSTLNQVS |
| 883 | AQVGRLGLA |
| 884 | AQWRPTAGG |
| 885 | AQYSRSPSS |
| 886 | AQNKRALMP |
| 887 | AQYSRNPAG |
| 888 | AQTFPSSRA |
| 889 | AQQVLSQVA |
| 890 | AQMKYTTAL |
| 891 | AQLKTAERA |
| 892 | AQVTSMHPG |
| 893 | AQMLRPNRS |
| 894 | AQVLNKGNH |
| 895 | AQGRKAYEH |
| 896 | AQCSSVCTL |
| 897 | AQNMTKMSA |
| 898 | AQLSPYEGD |
| 899 | AQTKYHLDL |
| 900 | AQYLCRGAL |
| 901 | AQSKHPAVY |
| 902 | AQSPSARPR |
| 903 | AQSQWAGPT |
| 904 | AQATRIPHL |
| 905 | AQVMNCCQH |
| 906 | AQKPGMLKV |
| 907 | AQVVFSQTS |
| 908 | AQNQTEKST |
| 909 | AQIVCGGLK |
| 910 | AQTVWQGGT |
| 911 | AQAKGMVSL |
| 912 | AQVTINQYG |
| 913 | AQNCGNNMW |
| 914 | AQDVRRLGI |
| 915 | AQILGKPDR |
| 916 | AQLQYEAME |
| 917 | AQRLSAQAQ |
| 918 | AQLSAGGLR |
| 919 | AQLHEKRLL |
| 920 | AQDASLVWK |
| 921 | AQQTPVARM |
| 922 | AQTMVGNGM |
| 923 | AQSIIAMGL |
| 924 | AQNARPVIK |
| 925 | AQSVVSRGM |
| 926 | AQTIMNQFA |
| 927 | AQRADNGIY |
| 928 | AQSQRAQTG |
| 929 | AQSPKVNKT |
| 930 | AQIYALSSG |
| 93 | AQLLCSDLV |
| 932 | AQWRSERNP |
| 933 | AQQHGIKGC |
| 934 | AQGHFVVFK |
| 935 | AQDNRIYRV |
| 936 | AQNMVGSGF |
| 937 | AQTKWHGDE |
| 938 | AQCDMSNMD |
| 939 | AQRPVRSEL |
| 940 | AQRVKDYAE |
| 941 | AQVICDQRN |
| 942 | AQSVIGYGT |
| 943 | AQSTPRSHG |
| 944 | AQANHHAIP |
| 945 | AQRPHVGIG |
| 946 | AQPNGPSAR |
| 947 | AQVHLRTEV |
| 948 | AQYLNGPVL |
| 949 | AQTATMQMS |
| 950 | AQDHLLPHP |
| 951 | AQLVESHLK |
| 952 | AQHTSFRLA |
| 953 | AQNYGANPV |
| 954 | AQDMLRLGM |
| 955 | AQGIIRLGA |
| 956 | AQGNNKTRP |
| 957 | AQVNTTKRI |
| 958 | AQSINYQVT |
| 959 | AQIVCLGRV |
| 960 | AQEARIRMC |
| 96 | AQVPTSPSN |
| 962 | AQAHQDQLC |
| 963 | AQKMVGYGE |
| 964 | AQGACYGAW |
| 965 | AQWKSPAAG |
| 966 | AQLTGRNKP |
| 967 | AQRQINIEK |
| 968 | AQEYTRSKV |
| 969 | AQTVVRNGT |
| 970 | AQNSSITYL |
| 97 | AQNPVKMSQ |
| 972 | AQLIGRYEL |
| 973 | AQGYTSSAK |
| 974 | AQYVTNQTS |
| 975 | AQNAQSANH |
| 976 | AQSAMYRTP |
| 977 | AQGPVRVNG |
| 978 | AQRVLTQVS |
| 979 | AQVICGLPK |
| 980 | AQLITGRLL |
| 981 | AQMGQPAMR |
| 982 | AQMSSLSYK |
| 983 | AQGLNPMCW |
| 984 | AQNTMVYAR |
| 985 | AQARVIDTR |
| 986 | AQNGTRMSA |
| 987 | AQNANRRDV |
| 988 | AQRFYPMKP |
| 989 | AQTFGTYSV |
| 990 | AQNVLQLGK |
| 991 | AQKQSNHLI |
| 992 | AQTLNFQSS |
| 993 | AQTTYNQMD |
| 994 | AQKWVKPGM |
| 995 | AQKTGEWCK |
| 996 | AQSRLTLKD |
| 997 | AQTISTHNT |
| 998 | AQSMTPSGS |
| 999 | AQTSTQRHS |
| 1000 | AQSFKNPLV |
| 1001 | AQVICASFI |
| 1002 | AQWSRMPTC |
| 1003 | AQSASRHRM |
| 1004 | AQRLPTSTA |
| 1005 | AQTIHYQIS |
| 1006 | AQSMVGPGN |
| 1007 | AQNQNPVRT |
| 1008 | AQSVLLQFS |
| 1009 | AQKLGHIVA |
| 1010 | AQSVRVSYN |
| 1011 | AQYAVSQRS |
| 1012 | AQFINSSGL |
| 1013 | AQRVTQSGE |
| 1014 | AQKGPYTHN |
| 1015 | AQVTMHSVK |
| 1016 | AQNVTHQSS |
| 1017 | AQFPLKVHG |
| 1018 | AQAGLSMRG |
| 1019 | AQFHCSMHP |
| 1020 | AQTFLHQIE |
| 1021 | AQSSSLPYL |
| 1022 | AQDRLQTRS |
| 1023 | AQFAGVTRP |
| 1024 | AQSNSRYCG |
| 1025 | AQRMREKLS |
| 1026 | AQKWHTSNC |
| 1027 | AQHFPLMVD |
| 1028 | AQRGNIDQG |
| 1029 | AQGPGFFRR |
| 1030 | AQIQHLSNV |
| 1031 | AQTHMAKAN |
| 1032 | AQDGPFLSP |
| 1033 | AQLTNVPLR |
| 1034 | AQTGYEYDS |
| 1035 | AQSPIARGE |
| 1036 | AQSPLRTGA |
| 1037 | AQVICTTEV |
| 1038 | AQAKHSLQS |
| 1039 | AQVTYNPIW |
| 1040 | AQWINGPPS |
| 1041 | AQSVVRSGG |
| 1042 | AQNRDINQS |
| 1043 | AQGVIALQT |
| 1044 | AQLLGMTKG |
| 1045 | AQVICDSRV |
| 1046 | AQLYIQTEK |
| 1047 | AQGVVALLS |
| 1048 | AQTVLNQND |
| 1049 | AQVICSNMS |
| 1050 | AQSMTQYGL |
| 1051 | AQQSPVGSR |
| 1052 | AQLHLGLLN |
| 1053 | AQAPTKLGS |
| 1054 | AQNPIQIGN |
| 1055 | AQTARLQVD |
| 1056 | AQGNWTKAS |
| 1057 | AQVTCSYMK |
| 1058 | AQVLTSHNT |
| 1059 | AQHVVKMSG |
| 1060 | AQHEPAYNF |
| 1061 | AQLSRLPHM |
| 1062 | AQSPIGQGR |
| 1063 | AQSPVGTAS |
| 1064 | AQLNTKFDS |
| 1065 | AQTPVKLSH |
| 1066 | AQTSAFVES |
| 1067 | AQAIPSHVR |
| 1068 | AQTQRLGIA |
| 1069 | AQTVIAFGD |
| 1070 | AQGPVRLAG |
| 1071 | AQVVCSATR |
| 1072 | AQSMTPNGG |
| 1073 | AQSQNPVQF |
| 1074 | AQNNSLAYT |
| 1075 | AQTVNVQIH |
| 1076 | AQTLQIQVT |
| 1077 | AQYLNSSGS |
| 1078 | AQITTSQLT |
| 1079 | AQYMTHQVN |
| 1080 | AQAMVKAGA |
| 1081 | AQGRLMRLL |
| 1082 | AQKLTPKNW |
| 1083 | AQQMYEREF |
| 1084 | AQNKGGSSS |
| 1085 | AQLRSFEES |
| 1086 | AQWYGAQVD |
| 1087 | AQYMNSSGS |
| 1088 | AQNDMRCVV |
| 1089 | AQVMQKTTA |
| 1090 | AQKMDTRYP |
| 1091 | AQKWDKLSS |
| 1092 | AQKYDPFDD |
| 1093 | AQGDFSPAG |
| 1094 | AQYVKYVEN |
| 1095 | AQGVVASMG |
| 1096 | AQQDPVRLK |
| 1097 | AQVEPRHKW |
| 1098 | AQTYWYAPL |
| 1099 | AQLICTHLI |
| 1100 | AQKGVMLYK |
| 1101 | AQTICSSLI |
| 1102 | AQRIGEYSV |
| 1103 | AQGVVKSGG |
| 1104 | AQISYGREF |
| 1105 | AQSWVSTSS |
| 1106 | AQVVCSGGR |
| 1107 | AQQMTQGGS |
| 1108 | AQSPVGIGS |
| 1109 | AQHRGMASL |
| 1110 | AQFKMPVDN |
| 1111 | AQLSSLSFK |
| 1112 | AQKASGTHP |
| 1113 | AQSTTYQMN |
| 1114 | AQEFGLSGC |
| 1115 | AQHIKNHAC |
| 1116 | AQNPVGARV |
| 1117 | AQVRTLRGG |
| 1118 | AQVVCSSWN |
| 1119 | AQSPMLCDK |
| 1120 | AQLCRASTR |
| 1121 | AQSTALSRK |
| 1122 | AQYVPNNKD |
| 1123 | AQRIHSSGL |
| 1124 | AQGVVALSS |
| 1125 | AQGIIRMDT |
| 1126 | AQAVVRAQG |
| 1127 | AQSPIRING |
| 1128 | AQTATWQIS |
| 1129 | AQHVIQMLG |
| 1130 | AQIVCAGVI |
| 1131 | AQYINSNAN |
| 1132 | AQGVVQDQG |
| 1133 | AQTDRADGV |
| 1134 | AQVATAQLN |
| 1135 | AQTLVCLTN |
| 1136 | AQMHRESGR |
| 1137 | AQGVQVGRL |
| 1138 | AQMSRPVFG |
| 1139 | AQTMLRSGA |
| 1140 | AQVPNAFSK |
| 1141 | AQSVSSGIS |
| 1142 | AQWKLYAGD |
| 1143 | AQTASFQTS |
| 1144 | AQIETMDLD |
| 1145 | AQSTCDTSR |
| 1146 | AQVLCTSVR |
| 1147 | AQVFATHNV |
| 1148 | AQTIFDGKK |
| 1149 | AQSSRVSSP |
| 1150 | AQPVTDKGR |
| 1151 | AQMRSVDRN |
| 1152 | AQLVCAYVM |
| 1153 | AQLLLRWVR |
| 1154 | AQTVKMCSG |
| 1155 | AQVICAGKP |
| 1156 | AQIVAGRSL |
| 1157 | AQTVFQMGS |
| 1158 | AQEVVKMGR |
| 1159 | AQTVVQNGA |
| 1160 | AQIVCSSLL |
| 1161 | AQMVVRSGE |
| 1162 | AQGIVRLGE |
| 1163 | AQGMSLLSK |
| 1164 | AQSPTKYGL |
| 1165 | AQTMTKRGE |
| 1166 | AQVVCGLVV |
| 1167 | AQYMMRLKM |
| 1168 | AQQGDNQLM |
| 1169 | AQTLKSNRD |
| 1170 | AQNSYKSNP |
| 1171 | AQYARGGSL |
| 1172 | AQAMTGMGA |
| 1173 | AQRSDLHHC |
| 1174 | AQSPYGLGN |
| 1175 | AQRQSDCGG |
| 1176 | AQCMSICSA |
| 1177 | AQGVTQMSH |
| 1178 | AQVVTLDRC |
| 1179 | AQNSRGPMN |
| 1180 | AQGALSIRV |
| 1181 | AQTLSTGIY |
| 1182 | AQLICSALQ |
| 1183 | AQPHMHVCI |
| 1184 | AQHPGACSH |
| 1185 | AQCGSRLML |
| 1186 | AQVVEAHTR |
| 1187 | AQMSNLVVS |
| 1188 | AQVGAISNA |
| 1189 | AQYRWLDPR |
| 1190 | AQSPFAMGT |
| 1191 | AQKSGLDKF |
| 1192 | AQQNPVAAR |
| 1193 | AQTIMVQMT |
| 1194 | AQRYMNGRL |
| 1195 | AQRKHGMAW |
| 1196 | AQFNKIDSP |
| 1197 | AQVHVRGSQ |
| 1198 | AQLYVNTER |
| 1199 | AQSSCVNTR |
| 1200 | AQKAGVMKA |
| 1201 | AQQMIRLSE |
| 1202 | AQSVLSHNI |
| 1203 | AQRLDHHTY |
| 1204 | AQHDDTFMS |
| 1205 | AQYVNSDGS |
| 1206 | AQKWLNLEV |
| 1207 | AQTVVKMVT |
| 1208 | AQSSAKCIK |
| 1209 | AQTAMSVDS |
| 1210 | AQGVIKMGQ |
| 1211 | AQSMLGLGR |
| 1212 | AQSPVKSGH |
| 1213 | AQFSTMKHL |
| 1214 | AQVKFIEPY |
| 1215 | AQVSCPEML |
| 1216 | AQVICMGLL |
| 1217 | AQAPIKLGE |
| 1218 | AQGFDPNKK |
| 1219 | AQEVYNPIW |
| 1220 | AQTYADQTD |
| 1221 | AQTIGAYMV |
| 1222 | AQEHTIDCV |
| 1223 | AQEVTRQLS |
| 1224 | AQVRRFLLW |
| 1225 | AQFLGQIKG |
| 1226 | AQTTIDGPC |
| 1227 | AQFRLYMSD |
| 1228 | AQSVIAWGD |
| 1229 | AQSIWHGVL |
| 1230 | AQQALLYKD |
| 1231 | AQKSSLSSM |
| 1232 | AQVRPTAAE |
| 1233 | AQVICSEVF |
| 1234 | AQSRSGSGA |
| 1235 | AQSNVTVDW |
| 1236 | AQDTAKRVG |
| 1237 | AQISQYLCS |
| 1238 | AQSIMERTD |
| 1239 | AQSRAQNPC |
| 1240 | AQVNATARL |
| 1241 | AQTIRTGIA |
| 1242 | AQVICGLIK |
| 1243 | AQLHLGKML |
| 1244 | AQTMTQMGS |
| 1245 | AQYRLSWHV |
| 1246 | AQPRTGHTT |
| 1247 | AQWAGAQAN |
| 1248 | AQYVSTLRA |
| 1249 | AQIQSPVGV |
| 1250 | AQSMGLAYK |
| 1251 | AQEPGLNRG |
| 1252 | AQCEGFLSG |
| 1253 | AQTYWYQPG |
| 1254 | AQCASLCSI |
| 1255 | AQLKICRGV |
| 1256 | AQCHVVSKG |
| 1257 | AQEGGDKGV |
| 1258 | AQGMSLLTR |
| 1259 | AQVYPKYFS |
| 1260 | AQDQRTSLH |
| 1261 | AQYRAADCS |
| 1262 | AQTGSGART |
| 1263 | AQSNRLSIP |
| 1264 | AQSGTNGMK |
| 1265 | AQVKQMVFK |
| 1266 | AQRNCPCDM |
| 1267 | AQLPVGWAS |
| 1268 | AQGEWIMEL |
| 1269 | AQRRNSVGS |
| 1270 | AQTMVRDGM |
| 1271 | AQGRETMLP |
| 1272 | AQVHTPCEG |
| 1273 | AQMGCGPLF |
| 1274 | AQEPPKNPK |
| 1275 | AQQKTVMYA |
| 1276 | AQVPCQGVM |
| 1277 | AQIVTRQVN |
| 1278 | AQLVTPKCG |
| 1279 | AQHTGDCAE |
| 1280 | AQLVCSNMM |
| 1281 | AQVGSNGDR |
| 1282 | AQPMVEMRK |
| 1283 | AQQLSVGPH |
| 1284 | AQGLYDLLI |
| 1285 | AQRWGSGPF |
| 1286 | AQMVQVATT |
| 1287 | AQHNSLSYM |
| 1288 | AQGVIREGN |
| 1289 | AQQMVIKMS |
| 1290 | AQTSQKNCR |
| 1291 | AQKFMEPNH |
| 1292 | AQVRLDSGR |
| 1293 | AQCGNERQF |
| 1294 | AQQYPEYRI |
| 1295 | AQLWDMNSP |
| 1296 | AQLLRVIFG |
| 1297 | AQMMKQCDT |
| 1298 | AQRSGISKH |
| 1299 | AQSPYNMGS |
| 1300 | AQVDKMIKG |
| 1301 | AQVEDFEPN |
| 1302 | AQSPPWSET |
| 1303 | AQMVSECHM |
| 1304 | AQTVVDDWP |
| 1305 | AQGILRLGL |
| 1306 | AQCFVTVRE |
| 1307 | AQGMKSLGS |
| 1308 | AQSNYSAAC |
| 1309 | AQSMVGLSY |
| 1310 | AQSTNGIQP |
| 1311 | AQNHASRVV |
| 1312 | AQFCPYIRM |
| 1313 | AQGRSMVHS |
| 1314 | AQQAVMTLS |
| 1315 | AQVTAKVQG |
| 1316 | AQFGVSLWA |
| 1317 | AQMLCTPAD |
| 1318 | AQGDSPYNS |
| 1319 | AQWCTDVHR |
| 1320 | AQSRTTRYV |
| 1321 | AQTFEGPLV |
| 1322 | AQLKGISKG |
| 1323 | AQERSMQRD |
| 1324 | AQLTPNADW |
| 1325 | AQKPLKDIL |
| 1326 | AQAQSFSSR |
| 1327 | AQQAPPYIL |
| 1328 | AQQRDCNGP |
| 1329 | AQGQNIPQK |
| 1330 | AQVCAKDRV |
| 1331 | AQGFKLKYN |
| 1332 | AQTNGTITT |
| 1333 | AQFKTVRGI |
| 1334 | AQCKNSAGI |
| 1335 | AQVIHECYI |
| 1336 | AQSVQVQIY |
| 1337 | AQNGTIPPC |
| 1338 | AQQMVRGGE |
| 1339 | AQLPGFGLS |
| 1340 | AQCTGKMEG |
| 1341 | AQTMFKNGE |
| 1342 | AQVLNRKCT |
| 1343 | AQECDRNGY |
| 1344 | AQSVQGDAA |
| 1345 | AQLVCSAIQ |
| 1346 | AQDVLSYRM |
| 1347 | AQRLSSAST |
| 1348 | AQNESYAYK |
| 1349 | AQSMFSQVE |
| 1350 | AQNPIKCGV |
| 1351 | AQVLVGGRG |
| 1352 | AQAVIRIGA |
| 1353 | AQRQVHVDC |
| 1354 | AQDYVECRG |
| 1355 | AQSATPTLR |
| 1356 | AQLYVHQLP |
| 1357 | AQMPYTSLE |
| 1358 | AQQSPIGMS |
| 1359 | AQRSNSALN |
| 1360 | AQLTLAGAE |
| 1361 | AQRVHLRVH |
| 1362 | AQPGRIRPP |
| 1363 | AQRDALKPS |
| 1364 | AQCDLGYRS |
| 1365 | AQNRKSNWC |
| 1366 | AQNENLQVS |
| 1367 | AQFANGICS |
| 1368 | AQTVLRLGE |
| 1369 | DGHQSRLGP |
| 1370 | DGRRIVPDK |
| 1371 | DGAISPHVA |
| 1372 | DGRPVVMLS |
| 1373 | DGTSTSRDR |
| 1374 | DGLISRHGA |
| 1375 | DGRSMGFNP |
| 1376 | DGAVSGMRN |
| 1377 | DGKAPNSGM |
| 1378 | DGGNNLQRK |
| 1379 | DGRALERKG |
| 1380 | DGRLMKIAS |
| 1381 | DGVNSSGPN |
| 1382 | DGKKNGLTV |
| 1383 | DGTAKELGL |
| 1384 | DGQWKGLRE |
| 1385 | DGRTMTSNR |
| 1386 | DGGVNKLGR |
| 1387 | DGDHPNNRK |
| 1388 | DGIRASRSD |
| 1389 | DGRDARTGY |
| 1390 | DGDTRALYS |
| 1391 | DGESVRPMR |
| 1392 | DGTAPRRPV |
| 1393 | DGEHTSELQ |
| 1394 | DGSRQKFGS |
| 1395 | DGRHTSKMV |
| 1396 | DGFKHSAMS |
| 1397 | DGLLNGRIQ |
| 1398 | DGTRRATPD |
| 1399 | DGNYKYVQG |
| 1400 | DGWMGGAAG |
| 1401 | DGIQYKTVG |
| 1402 | DGQYRDKST |
| 1403 | DGVNLSRGH |
| 1404 | DGHLPRWNP |
| 1405 | DGLSLMALR |
| 1406 | DGWNKVQAR |
| 1407 | DGHDAGWRG |
| 1408 | DGYASMKTL |
| 1409 | DGMNKKAGG |
| 1410 | DGFVKKVYG |
| 1411 | DGLQPVRAR |
| 1412 | DGIGHLVGG |
| 1413 | DGYRSPNVK |
| 1414 | DGRMRTDLY |
| 1415 | DGVSKLYKI |
| 1416 | DGSQKMSVR |
| 1417 | DGRPSPNRA |
| 1418 | DGRASSTER |
| 1419 | DGTPLHDNV |
| 1420 | DGYKGQAMR |
| 1421 | DGAAKYRSP |
| 1422 | DGRGSHSPG |
| 1423 | DGRNNPIWA |
| 1424 | DGNNAQRPS |
| 1425 | DGKIAMVKG |
| 1426 | DGMDVGRYG |
| 1427 | DGGMSPVWK |
| 1428 | DGHAQRKDY |
| 1429 | DGRESCKGT |
| 1430 | DGCTYSRMP |
| 1431 | DGCLEGAMY |
| 1432 | DGLGSMSSW |
| 1433 | DGALKCRSE |
| 1434 | DGSWTAICS |
| 1435 | DGSKPSRIT |
| 1436 | DGRQGKSRI |
| 1437 | DGRCIDYEV |
| 1438 | DGEEAPWKM |
| 1439 | DGSVVSGMQ |
| 1440 | DGADNGLHS |
| 1441 | DGSCLSSAI |
| 1442 | DGGSVTKWI |
| 1443 | DGDGVKDSQ |
| 1444 | DGCNVVYPR |
| 1445 | DGVFQGSTV |
| 1446 | DGLRCPDAE |
| 1447 | DGPLCSRRR |
| 1448 | DGHNWGRAE |
| 1449 | DGSVDPCRT |
| 1450 | DGFWHDMRS |
| 1451 | DGEEDYVFY |
| 1452 | DGRGDSVCM |
| 1453 | DGRNTMLVS |
| 1454 | DGRQGLSQC |
| 1455 | DGVHACPQA |
| 1456 | DGVIKRQCA |
| 1457 | DGACETLFV |
| 1458 | DGLPGPFDC |
| 1459 | DGVAKEPFL |
| 1460 | DGGICLLAS |
| 1461 | DGRSRKPMT |
| 1462 | DGYDCPSRG |
| 1463 | DGQNSLSFK |
| 1464 | DGAYRSCAS |
| 1465 | DGCKSQLAR |
| 1466 | DGAQDIALF |
| 1467 | DGEINTYCM |
| 1468 | DGSDSWGDN |
| 1469 | DGLRYPDPE |
| 1470 | DGWLASDES |
| 1471 | DGYLSSVPG |
| 1472 | DGVVRCLSF |
| 1473 | DGCKIGTIA |
| 1474 | DGLVGGVCS |
| 1475 | DGDLRAVRM |
| 1476 | DGAECRFDN |
| 1477 | DGSCLVERF |
| 1478 | DGERIRIFD |
| 1479 | DGSRPAFCL |
| 1480 | DGAWVNTHE |
| 1481 | DGTMPAPCS |
| 1482 | DGRGFPEGN |
| 1483 | DGWGPNRCD |
| 1484 | DGRPVVHTY |
| 1485 | DGVAHDQVP |
| 1486 | DGCCAGIGT |
| 1487 | DGYRNLGYW |
| 1488 | DGLGNLPCR |
| 1489 | DGMVDPHLY |
| 1490 | DGWTEYDNN |
| 1491 | DGGFFVTDE |
| 1492 | DGLSGGGGK |
| 1493 | DGKVGMIMP |
| 1494 | DGGVGLYYL |
| 1495 | DGVYKMLDR |
| 1496 | DGGGSFLNW |
| 1497 | DGYKWYRPT |
| 1498 | DGSYIICCP |
| 1499 | DGQRVTKLP |
| 1500 | DGFARRSVQ |
| 1501 | DGWLSLLLA |
| 1502 | DGDRREVLC |
| 1503 | DGARVSRIK |
| 1504 | DGFAYLDPD |
| 1505 | DGIYEDWKS |
| 1506 | DGGGCTLVD |
| 1507 | DGRKPHGDG |
| 1508 | DGHRANGNL |
| 1509 | DGLNPTTIS |
| 1510 | DGPHGAVDG |
| 1511 | DGGHCESSS |
| 1512 | DGWMISPKK |
| 1513 | DGWCDIHQF |
| 1514 | DGLNRRSNV |
| 1515 | DGLVVGHAG |
| 1516 | DGSYDLEGF |
| 1517 | DGNPEKCIS |
| 1518 | DGNHTFYEG |
| 1519 | DGYSPTWLA |
| 1520 | DGDMCKAEF |
| 1521 | DGCSSSSLY |
| 1522 | DGVHVSIAC |
| 1523 | DGYFLDVQC |
| 1524 | DGDPRRCLE |
| 1525 | DGWDMGRSG |
| 1526 | DGCRSGGAR |
| 1527 | DGVKCFSEF |
| 1528 | DGCGEPERC |
| 1529 | DGFRQTVCG |
| 1530 | DGTQTNIHH |
| 1531 | DGMRRMKDN |
| 1532 | DGSATNLVR |
| 1533 | DGDPLYRVH |
| 1534 | DGTSCFTAS |
| 1535 | DGCEDTVYG |
| 1536 | DGLEFFSIV |
| 1537 | DGTDTVCRG |
| 1538 | DGGAQMVEW |
| 1539 | DGLATMPLC |
| 1540 | DGNTGWFDG |
| 1541 | DGNPMSAFP |
| 1542 | DGRSLAFPY |
| 1543 | DGRHCWSSF |
| 1544 | DGEVCERLR |
| 1545 | DGCAVMVEP |
| 1546 | DGQPLVLAV |
| 1547 | DGSRRLKLK |
| 1548 | DGMPQGRPP |
| 1549 | DGAAVRCHV |
| 1550 | DGKGVTRSY |
| 1551 | DGLKNRMLS |
| 1552 | DGSLASEGR |
| 1553 | DGLRRPLRV |
| 1554 | DGVGSKRVR |
| 1555 | DGESSDKGS |
| 1556 | DGTGGRLLP |
| 1557 | DGVCKSLQK |
| 1558 | DGRSQPIAW |
| 1559 | DGRTFLADM |
| 1560 | DGEATCKRV |
| 1561 | DGRKVTKAN |
| 1562 | DGDLRHPSC |
| 1563 | DGNATHGRR |
| 1564 | DGHSYQCDL |
| 1565 | DGMSAQSTG |
| 1566 | DGASSICRK |
| 1567 | DGLCKAKWI |
| 1568 | DGKGGSCHS |
| 1569 | DGLAVGIGR |
| 1570 | DGSKARTYR |
| 1571 | DGCTSYVSP |
| 1572 | DGIVGIYKG |
| 1573 | DGFSGDRTL |
| 1574 | DGFSQKKKS |
| 1575 | DGRLQMCRV |
| 1576 | DGKKCNLMH |
| 1577 | DGTGKLRAW |
| 1578 | DGCSNWMFT |
| 1579 | DGLGFKAPR |
| 1580 | DGKRASGKV |
| 1581 | DGYSGTVCI |
| 1582 | DGTLVRLRL |
| 1583 | DGHISRFLR |
| 1584 | DGNDHRYSG |
| 1585 | DGGKQKVCS |
| 1586 | DGSVTPKNQ |
| 1587 | DGKLSGHHA |
| 1588 | DGMHCKLPS |
| 1589 | DGRAPAARK |
| 1590 | DGFCGEFNT |
| 1591 | DGDHPAGMR |
| 1592 | DGSSGLCGT |
| 1593 | DGRAVGSVW |
| 1594 | DGLAPCIYK |
| 1595 | DGGLRALPS |
| 1596 | DGGLSRLCI |
| 1597 | DGRTYGTKR |
| 1598 | DGAVAHSLE |
| 1599 | DGMYSWGAD |
| 1600 | DGASDWHLD |
| 1601 | DGISFHIAF |
| 1602 | DGPVADGSH |
| 1603 | DGHLSAYRS |
| 1604 | DGFRGANMP |
| 1605 | DGHWLAAQC |
| 1606 | DGYDEYMHV |
| 1607 | DGECDVYVY |
| 1608 | DGSMSPHKG |
| 1609 | DGVSLCPDG |
| 1610 | DGIDALCSF |
| 1611 | DGFTSGPIH |
| 1612 | DGQSSESES |
| 1613 | DGLWYGEGT |
| 1614 | DGATNRPIQ |
| 1615 | DGRRCAEGA |
| 1616 | DGIEWWGLA |
| 1617 | DGGQCTGGP |
| 1618 | DGHSYVMKV |
| 1619 | DGYNRTMWS |
| 1620 | DGYCQDYYW |
| 1621 | DGKKATSKL |
| 1622 | DGWLCADVM |
| 1623 | DGSSFCNIL |
| 1624 | DGPTVESAN |
| 1625 | DGHPVRNCK |
| 1626 | DGFIDSGCA |
| 1627 | DGVRLWHMA |
| 1628 | DGTVTMLWV |
| 1629 | DGDGERRCS |
| 1630 | DGIIMRRKS |
| 1631 | DGVNSPSLF |
| 1632 | DGVEMPRSE |
| 1633 | DGVLSRRSG |
| 1634 | DGWRTDRPD |
| 1635 | DGTVCSSGF |
| 1636 | DGEPLWFPS |
| 1637 | DGCMHGILW |
| 1638 | DGREKCFRE |
| 1639 | DGTGRGLTL |
| 1640 | DGGSGCGKN |
| 1641 | DGGRNVGGF |
| 1642 | DGMSFCNKM |
| 1643 | DGGRAGNSG |
| 1644 | DGSNGCITE |
| 1645 | DGQYTEWKH |
| 1646 | DGLKALGLN |
| 1647 | DGTCCNPLP |
| 1648 | DGYAPLGRA |
| 1649 | DGRLDRYTS |
| 1650 | DGILVFLDL |
| 1651 | DGDLLGGPL |
| 1652 | DGRSKDFDT |
| 1653 | DGAKGFDVH |
| 1654 | DGRVRRLGS |
| 1655 | DGLWVHARL |
| 1656 | DGARARRCG |
| 1657 | DGPMGWELA |
| 1658 | DGRILGLMT |
| 1659 | DGHHGNEPW |
| 1660 | DGGSRCGAL |
| 1661 | DGCKRAQES |
| 1662 | DGPYGIMGS |
| 1663 | DGPTGYLVK |
| 1664 | DGRSNDLCD |
| 1665 | DGLRPSDIR |
| 1666 | DGYERFEPG |
| 1667 | DGSIRRFVC |
| 1668 | DGIVNFNVR |
| 1669 | DGLGRPLVS |
| 1670 | DGDSQPWKL |
| 1671 | DGKSRCTDN |
| 1672 | DGSCAPGAM |
| 1673 | DGNIPRSAG |
| 1674 | DGVGTSRMF |
| 1675 | DGFNWLKCY |
| 1676 | DGLFCLKTL |
| 1677 | DGGRGLGVL |
| 1678 | DGRIIFQVK |
| 1679 | DGDLHYVKS |
| 1680 | DGWACPRSR |
| 1681 | DGDQSDLTW |
| 1682 | DGFLNCSMT |
| 1683 | DGYLSEYVR |
| 1684 | DGVWLNMVP |
| 1685 | DGYLWYRDD |
| 1686 | DGGAPEGIR |
| 1687 | DGEKTPGIL |
| 1688 | DGLDFYSIS |
| 1689 | DGWVRSSVL |
| 1690 | DGLHHTPCG |
| 1691 | DGSGKRGIY |
| 1692 | DGNEPRCRV |
| 1693 | DGRGKEKND |
| 1694 | DGNECSEVE |
| 1695 | DGKKNMSGF |
| 1696 | DGFDCRWSM |
| 1697 | DGNSPRACS |
| 1698 | DGNSNTFPA |
| 1699 | DGADLGRRH |
| 1700 | DGLVNRIYG |
| 1701 | DGFVDVAAG |
| 1702 | DGWSCSLKR |
| 1703 | DGTLVIKTC |
| 1704 | DGPRTSEYC |
| 1705 | DGRGPSVGC |
| 1706 | DGSQSGLVV |
| 1707 | DGRGNADIR |
| 1708 | DGRCMTGIT |
| 1709 | DGAGPSCVN |
| 1710 | DGEVNVGGH |
| 1711 | DGRDRKSCN |
| 1712 | DGNNIVVFD |
| 1713 | DGLWQEILD |
| 1714 | DGNNHCRVY |
| 1715 | DGHPGVTKG |
| 1716 | DGCMVSGQD |
| 1717 | DGGHNCLGY |
| 1718 | DGRPAGVDS |
| 1719 | AQSVTPKNQ |
| 1720 | AQSMLKLGE |
| 1721 | DGDYRTVKQ |
| 1722 | AQYPPVFKS |
| 1723 | AQELKVKQQ |
| 1724 | AQWTLNQTS |
| 1725 | AQALPSHHS |
| 1726 | AQKRAGPST |
| 1727 | AQQVIKGGE |
| 1728 | AQTVLRSGE |
| 1729 | AQQSPIQSL |
| 1730 | AQSVIGMSG |
| 1731 | DGQSVVKLG |
| 1732 | AQSPVGLGE |
| 1733 | AQTENNQLD |
| 1734 | DGSGPIRMG |
| 1735 | AQAMVGMGV |
| 1736 | AQSVVGLGA |
| 1737 | AQTVVGMGI |
| 1738 | AQSTVMQIS |
| 1739 | AQHTLSQMS |
| 1740 | AQRKEKERW |
| 1741 | AQSITGLGY |
| 1742 | AQFATMQVS |
| 1743 | DGQDPVRKT |
| 1744 | AQSSSRLCL |
| 1745 | AQKYGKYTS |
| 1746 | AQIVTNQYG |
| 1747 | AQAMVQMLG |
| 1748 | AQMMVGLGQ |
| 1749 | AQTVQIQTY |
| 1750 | AQEGSFPRR |
| 1751 | AQGYGKRTY |
| 1752 | AQSIMRLGE |
| 1753 | AQGMIGMGS |
| 1754 | AQTVLHMGE |
| 1755 | DGVWLNIET |
| 1756 | AQSIVRLGD |
| 1757 | AQSIFRNGE |
| 1758 | AQTVVQYGE |
| 1759 | AQKPTKLGE |
| 1760 | DGGGSMSTW |
| 1761 | AQGPIRDGA |
| 1762 | DGVVGHVKG |
| 1763 | DGFRFQDRD |
| 1764 | DGDNYVTKP |
| 1765 | DGKDGHWKP |
| 1766 | AQSVVKLGT |
| 1767 | DGRVTREFD |
| 1768 | AQGKYTAWE |
| 1769 | AQTVVKLGS |
| 1770 | AQSVVKMSD |
| 1771 | AQSVHYQVS |
| 1772 | DGKTGIMKP |
| 1773 | AQIICSSVR |
| 1774 | DGLSPGRFL |
| 1775 | AQNVLQMGK |
| 1776 | AQSTTAQYN |
| 1777 | DGLIYEYEH |
| 1778 | DGDNRVTRE |
| 1779 | AQMPIRDGQ |
| 1780 | AQTVVAQKA |
| 1781 | DGDNYILKG |
| 1782 | AQHFPRMVD |
| 1783 | DGSHMLLHV |
| 1784 | DGDFYLLKP |
| 1785 | DGKYEPPAC |
| 1786 | AQGTYMATD |
| 1787 | AQGMIKGGS |
| 1788 | AQSTTWQTS |
| 1789 | DGDGYLYKG |
| 1790 | AQYRNGGAS |
| 1791 | DGDEYFERP |
| 1792 | DGKVGQTKT |
| 1793 | DGKRGMDKP |
| 1794 | DGFIYDYKH |
| 1795 | DGLNWHTID |
| 1796 | DGKAGVMKA |
| 1797 | AQGKYSGIE |
| 1798 | AQGVVAYLT |
| 1799 | DGDNYHVPP |
| 1800 | AQGDSLPYR |
| 1801 | AQWPTSYDD |
| 1802 | DGILGLFKG |
| 1803 | AQLVAGRDL |
| 1804 | DGDNYFLKV |
| 1805 | DGDNYLLKT |
| 1806 | DGVPGHHKS |
| 1807 | DGGKYTYTE |
| 1808 | AQGVIGIKG |
| 1809 | AQERCCPLV |
| 1810 | AQSKYTAND |
| 1811 | DGGVVAIYR |
| 1812 | DGFSHAALW |
| 1813 | AQVVCNSIK |
| 1814 | AQLTRALLR |
| 1815 | DGDNYRTKG |
| 1816 | AQSGPIRMG |
| 1817 | DGDNYSHLP |
| 1818 | DGDKYFYKE |
| 1819 | DGDNYKYVG |
| 1820 | AQKVTGFGS |
| 1821 | AQCVSLCSA |
| 1822 | DGDDYFVKE |
| 1823 | AQGKYTHND |
| 1824 | AQGRYVPGD |
| 1825 | DGRAGQNKG |
| 1826 | AQSVVRLGV |
| 1827 | DGLSLWVSG |
| 1828 | DGDNYLIKK |
| 1829 | AQVICSNTV |
| 1830 | AQTPIRMGP |
| 1831 | DGCNGIEFG |
| 1832 | AQGVVRMAQ |
| 1833 | DGLCNMFRY |
| 1834 | DGKVGMIKL |
| 1835 | AQGMYTYLD |
| 1836 | AQMRPGPTD |
| 1837 | AQLCIADWI |
| 1838 | DGDYRVTKQ |
| 1839 | DGLICSYNG |
| 1840 | AQYYECSQQ |
| 1841 | DGLDSVCPS |
| 1842 | AQGNSILWL |
| 1843 | AQVWRTSTS |
| 1844 | AQGRYTSWA |
| 1845 | AQVICSPAI |
| 1846 | AQCVAAMKC |
| 1847 | AQLICTTTN |
| 1848 | DGCRCDMVC |
| 1849 | AQSEKGYCL |
| 1850 | DGTYCSQES |
| 1851 | AQKMHVDRW |
| 1852 | DGKSPHYKP |
| 1853 | DGLKLISKG |
| 1854 | AQGPVKMGF |
| 1855 | DGHTCSPNT |
| 1856 | AQSRCSDET |
| 1857 | AQYYYLFNS |
| 1858 | AQVYDLVFC |
| 1859 | AQQNPIAVR |
| 1860 | AQIVCDHRI |
| 1861 | AQLLLTCLF |
| 1862 | AQTAMEFDH |
| 1863 | DGVVHETVR |
| 1864 | DGGDINYNW |
| 1865 | AQDKPGQVK |
| 1866 | AQPSSLSYK |
| 1867 | DGEVGLTVR |
| 1868 | DGIVGSTIR |
| 1869 | AQRETTAFI |
| 1870 | DGVVGVNIR |
| 1871 | AQTPVGLGE |
| 1872 | AQRETNAFI |
| 1873 | AQHNVMSTQ |
| 1874 | DGIFGGGGK |
| 1875 | AQRWKQEIE |
| 1876 | AQLEYARSL |
| 1877 | DGIKGGGSK |
| 1878 | DGIVHLAVR |
| 1879 | AQVICSSLI |
| 1880 | DGFGFTLST |
| 1881 | AQVICSYIV |
| 1882 | DGRPYGPVL |
| 1883 | AQKSAGFAS |
| 1884 | AQTKSNGIS |
| 1885 | AQERYTSHN |
| 1886 | AQQDAKVTT |
| 1887 | DGLCIADWI |
| 1888 | DGVICSYIV |
| 1889 | AQFYPSSSK |
| 1890 | AQTMLRMGE |
| 1891 | DGNEVVWRV |
| 1892 | AQKISVMYE |
| 1893 | AQTISHQIS |
| 1894 | DGLTYYGIE |
| 1895 | AQQDGVAEG |
| 1896 | DGAPFGPAI |
| 1897 | AQSVLSQVS |
| 1898 | DGVWLNLSS |
| 1899 | AQSVSTGIS |
| 1900 | AQSPFKEGL |
| 1901 | AQTVLFQQS |
| 1902 | AQCGSGHIE |
| 1903 | DGQSPIMRM |
| 1904 | AQTWDTGIS |
| 1905 | AQTVLFQVE |
| 1906 | DGHGGGSRY |
| 1907 | AQTTLSQQS |
| 1908 | AQTVNYQVT |
| 1909 | AQVICSSVV |
| 1910 | DGNLSMRSE |
| 1911 | AQPMLDRNT |
| 1912 | AQTTLSQVT |
| 1913 | DGIAEEDWM |
| 1914 | AQNQSPVGF |
| 1915 | DGMGPIRMS |
| 1916 | AQVICMGII |
| 1917 | AQHTTHQVS |
| 1918 | DGLRATSPS |
| 1919 | AQTIWSGDM |
| 1920 | AQENDMVFF |
| 1921 | DGLLEYTFR |
| 1922 | AQTVLHQTH |
| 1923 | AQSISTGIY |
| 1924 | AQTTVHQSS |
| 1925 | AQTTLQQIT |
| 1926 | AQUCSSLR |
| 1927 | AQTVNFQIT |
| 1928 | DGVPSVDIR |
| 1929 | AQFIASHNV |
| 1930 | DGCQPPKCI |
| 1931 | AQVVCSHTL |
| 1932 | AQGVIRMGD |
| 1933 | AQLICYDKN |
| 1934 | AQDYPFVDP |
| 1935 | DGTQSPTSW |
| 1936 | AQSVTKLGA |
| 1937 | AQTRFIEPY |
| 1938 | AQILRIEPT |
| 1939 | AQEHTSELQ |
| 1940 | DGVVCSYNR |
| 1941 | AQVVCSGVI |
| 1942 | AQPVDYKPW |
| 1943 | AQLVCSHLR |
| 1944 | AQNPVRNGA |
| 1945 | AQTVTKQIN |
| 1946 | AQGRYTASE |
| 1947 | AQSMVKGGS |
| 1948 | DGKTGLSKP |
| 1949 | AQGFDPNNR |
| 1950 | AQTTFLQID |
| 1951 | AQTMVKGGP |
| 1952 | AQTVVKMGV |
| 1953 | AQVICSSVM |
| 1954 | AQLSAFTSA |
| 1955 | AQNPTKMGM |
| 1956 | DGFGITWTE |
| 1957 | AQGQSLSYM |
| 1958 | AQTFVSHNI |
| 1959 | AQSVVRDKG |
| 1960 | AQSIHSHNT |
| 1961 | DGMVAEWVG |
| 1962 | AQSMVKGGP |
| 1963 | DGGSYKIIV |
| 1964 | AQTRMSGIS |
| 1965 | AQGRQDCSD |
| 1966 | AQSTTYQFS |
| 1967 | AQEVGLTVR |
| 1968 | AQDGSLTFR |
| 1969 | AQGKYAAIC |
| 1970 | AQKDGHNKG |
| 1971 | AQGVQRRMH |
| 1972 | AQELKGKCG |
| 1973 | AQMDGLWKG |
| 1974 | AQCKPLNSR |
| 1975 | AQSYKEYNL |
| 1976 | AQLICDYRI |
| 1977 | AQTVRNQVS |
| 1978 | AQPRLGLAA |
| 1979 | AQVICANTR |
| 1980 | DGSLIFGMR |
| 1981 | AQVLCTTLK |
| 1982 | AQNVIKLGM |
| 1983 | DGTVFFICL |
| 1984 | AQGICATQR |
| 1985 | AQVICTNVR |
| 1986 | AQESLSSAQ |
| 1987 | AQTKGSGIS |
| 1988 | AQSVTKFGA |
| 1989 | AQTTLRQVT |
| 1990 | DGRIGHSKP |
| 1991 | AQDRPLYVY |
| 1992 | DGDWPYCVF |
| 1993 | AQLVEGHWK |
| 1994 | AQCGLGANC |
| 1995 | AQTVISQRS |
| 1996 | AQSPVKLGY |
| 1997 | AQQHVNLFV |
| 1998 | AQLYVNELR |
| 1999 | AQLVNYSML |
| 2000 | AQTTLSQRT |
| 2001 | DGRLWLGGV |
| 2002 | AQGSSLPFK |
| 2003 | AQTLQYQKS |
| 2004 | AQTKIYSKS |
| 2005 | AQRSMFSHG |
| 2006 | AQGNSLKYT |
| 2007 | DGHDYCHFF |
| 2008 | DGMCNAMIP |
| 2009 | AQTRVFTVI |
| 2010 | DGPIIERFV |
| 2011 | AQFYTSHNT |
| 2012 | DGLRCSNQF |
| 2013 | AQLNSLSFK |
| 2014 | AQMLPSYVR |
| 2015 | AQQNSLSFK |
| 2016 | AQEYTVPRW |
| 2017 | AQTRWHGNE |
| 2018 | AQNYLDYCA |
| 2019 | AQSRSLSSL |
| 2020 | AQMICTYVK |
| 2021 | DGWSCCGRR |
| 2022 | AQLNRLPHL |
| 2023 | AQKLSSHNT |
| 2024 | AQTKQLGIY |
| 2025 | DGGVTWLRM |
| 2026 | AQNLSLAYK |
| 2027 | AQKYSLSSM |
| 2028 | AQVYEVPRW |
| 2029 | AQPWLPDNL |
| 2030 | AQNNSLSYR |
| 2031 | AQGKSLAWT |
| 2032 | AQLNDIRRH |
| 2033 | AQFKIYAAQ |
| 2034 | DGGAYWSLF |
| 2035 | AQINDLRRY |
| 2036 | AQKVYTGIS |
| 2037 | AQRMVIPSH |
| 2038 | AQGSSMMYK |
| 2039 | AQGMSLKYQ |
| 2040 | AQRIHLSSI |
| 2041 | AQNHQISGS |
| 2042 | AQGKAANNA |
| 2043 | DGIYSCPSG |
| 2044 | AQTFGNPVS |
| 2045 | AQRIMSHNV |
| 2046 | AQYPAISFK |
| 2047 | DGVVGSTVR |
| 2048 | DGIVSSTVR |
| 2049 | DGIPHSTIR |
| 2050 | DGIVGINIR |
| 2051 | DGIVHMIVR |
| 2052 | DGIVHLNIR |
| 2053 | DGIPLSTIR |
| 2054 | DGEVGLTTR |
| 2055 | DGIVDLAER |
| 2056 | DGEVHETVR |
| 2057 | DGEVESTIR |
| 2058 | DGYVGGTIR |
| 2028 | AQVYEVPRW |
| 2029 | AQPWLPDNL |
| 2030 | AQNNSLSYR |
| 2031 | AQGKSLAWT |
| 2032 | AQLNDIRRH |
| 2033 | AQFKIYAAQ |
| 2034 | DGGAYWSLF |
| 2035 | AQINDLRRY |
| 2036 | AQKVYTGIS |
| 2037 | AQRMVIPSH |
| 2038 | AQGSSMMYK |
| 2039 | AQGMSLKYQ |
| 2040 | AQRIHLSSI |
| 2041 | AQNHQISGS |
| 2042 | AQGKAANNA |
| 2043 | DGIYSCPSG |
| 2044 | AQTFGNPVS |
| 2045 | AQRIMSHNV |
| 2046 | AQYPAISFK |
| 2047 | DGVVGSTVR |
| 2048 | DGIVSSTVR |
| 2049 | DGIPHSTIR |
| 2050 | DGIVGINIR |
| 2051 | DGIVHMIVR |
| 2052 | DGIVHLNIR |
| 2053 | DGIPLSTIR |
| 2054 | DGEVGLTTR |
| 2055 | DGIVDLAER |
| 2056 | DGEVHETVR |
| 2057 | DGEVESTIR |
| 2058 | DGYVGGTIR |
| 2042 | AQGKAANNA |
| 2043 | DGIYSCPSG |
| 2044 | AQTFGNPVS |
| 2045 | AQRIMSHNV |
| 2046 | AQYPAISFK |
| 2047 | DGVVGSTVR |
| 2048 | DGIVSSTVR |
| 2049 | DGIPHSTIR |
| 2050 | DGIVGINIR |
| 2051 | DGIVHMIVR |
| 2052 | DGIVHLNIR |
| 2053 | DGIPLSTIR |
| 2054 | DGEVGLTTR |
| 2055 | DGIVDLAER |
| 2056 | DGEVHETVR |
| 2057 | DGEVESTIR |
| 2058 | DGYVGGTIR |

In certain embodiments, the 9-mer amino acid peptides that bind to rhesus CA4 (rhCA4) are provided in Table 5. In certain preferred embodiments, the 9-mer amino acid peptides in Table 5 are after position 586 of AAV9 capsid protein or a functional equivalent thereof.

TABLE 5
Rhesus CA-IV binding 9-mer amino acid peptides

| SEQ | |
|---|---|
| ID NO | 9-mer |
| 2059 | AQDVTRQSG |
| 2060 | AQSRSLSSL |
| 2061 | AQSKDAKYP |
| 2062 | AQDKPGQGK |
| 2063 | AQLADAMQC |
| 2064 | AQMFCRWVR |
| 2065 | AQVRIRRLM |
| 2066 | AQTRKTSPV |
| 2067 | AQHVTAQTY |
| 2068 | AQILVLLIR |
| 2069 | AQLMPSTNL |
| 2070 | AQRFTKLLK |
| 2071 | AQSKRSLEL |
| 2072 | AQQHSLSFK |
| 2073 | AQIFTKNST |
| 2074 | AQPGNGKRG |
| 2075 | AQIHKGIAI |
| 2076 | AQSPLRPTD |
| 2077 | AQNGMGGVS |
| 2078 | AQQDSLRFK |
| 2079 | AQDIKGMGI |
| 2080 | AQMGLLRGP |
| 2081 | AQNLRLPCL |
| 2082 | AQTMIANGS |
| 2083 | AQQKSLRFK |
| 2084 | AQGDSLPYR |
| 2085 | AQSNVMPCG |
| 2086 | AQRAEPRTL |
| 2087 | AQYDCYTED |
| 2088 | AQSVVSMGR |
| 2089 | AQAGGMGSK |
| 2090 | AQSFISETA |
| 2091 | AQFNSISSK |
| 2092 | AQSSARSAS |
| 2093 | AQRPTHVRD |
| 2094 | AQGRRIVSN |
| 2095 | AQELPYVSG |
| 2096 | AQESSFTKE |
| 2097 | AQMPRDGCA |
| 2098 | AQNASGMSM |
| 2099 | AQQRDDTVA |
| 2100 | AQKNLRWGH |
| 2101 | AQISNYPKR |
| 2102 | AQLLVKELS |
| 2103 | AQHSQLRGV |
| 2104 | AQLRGLPSC |
| 2105 | AQWRTMQRR |
| 2106 | AQKDLGRDE |
| 2107 | AQSMYQMGS |
| 2108 | AQSPYKQGL |
| 2109 | AQFPKGTIA |
| 2110 | AQVRNRAGV |
| 2111 | AQRSINDTR |
| 2112 | AQLVVDSWT |
| 2113 | AQKCVSIAA |
| 2114 | AQTASSPRH |
| 2115 | AQGVVSCRT |
| 2116 | AQGKAYPVV |
| 2117 | AQGESAFAT |
| 2118 | AQHHRRSGM |
| 2119 | AQTYNDYRA |
| 2120 | AQEGVECNR |
| 2121 | AQLVLKSSG |
| 2122 | AQAQGSTHT |
| 2123 | AQNIATNRS |
| 2124 | AQTLSRTCL |
| 2125 | AQDMRKTVF |
| 2126 | AQKDRVIPN |
| 2127 | AQTRPQPGT |
| 2128 | AQVLSRMHC |
| 2129 | AQWRSYVLD |
| 2130 | AQNRLCKIT |
| 2131 | AQRGAIVLG |
| 2132 | AQVSCMSKL |
| 2133 | AQCVSNAWQ |
| 2134 | AQRQGSPSP |
| 2135 | AQLKCGRTL |
| 2136 | AQTSTLVFH |
| 2137 | AQHMCDSRM |
| 2138 | AQFKSLHCA |
| 2139 | AQDVYTSRG |
| 2140 | AQLLTVSEC |
| 2141 | AQVECTTLM |
| 2142 | AQLLFVFRM |
| 2143 | AQACWPTFC |
| 2144 | AQLCSLYEV |
| 2145 | AQNPNASLN |
| 2146 | AQMEVRACM |
| 2147 | AQQACRSLI |
| 2148 | AQTFVKMVE |
| 2149 | AQLPAGTER |
| 2150 | AQHFSTQMT |
| 2151 | AQQNSLRLK |
| 2152 | AQYVIIGKS |
| 2153 | AQEGIDKET |
| 2154 | AQTGCMAVD |
| 2155 | AQGFHNMHG |
| 2156 | AQTRSNATG |
| 2157 | AQMVKCTVS |
| 2158 | AQPLVASGR |
| 2159 | AQTEHALCW |
| 2160 | AQRMNDSNR |
| 2161 | AQLVTRQTS |
| 2162 | AQRYVQGGE |
| 2163 | AQTNASCTY |
| 2164 | AQILNLNKG |
| 2165 | AQSRIGRGC |
| 2166 | AQPAIFPEC |
| 2167 | AQSASTISA |
| 2168 | AQTTGSALP |
| 2169 | AQRSMCTAE |
| 2170 | AQNGETSRL |
| 2171 | AQRTFRSNL |
| 2172 | AQQPHLAAP |
| 2173 | AQTGSPTIV |
| 2174 | AQEFDRNNS |
| 2175 | AQRVKKVPD |
| 2176 | AQPYKANKF |
| 2177 | AQSSSHLRS |
| 2178 | AQNRLVSEL |
| 2179 | AQPEKQACV |
| 2180 | AQLSAFHEK |
| 2181 | AQSTWPHVS |
| 2182 | AQTSSPPTM |
| 2183 | AQTQDFISK |
| 2184 | AQGVVALGM |
| 2185 | AQCVGRERD |
| 2186 | AQCVMNVSC |
| 2187 | AQFTSSQMN |
| 2188 | AQKTCSSDT |
| 2189 | AQKGHTAPG |
| 2190 | AQLKAGAQA |
| 2191 | AQSILASVP |
| 2192 | AQFPSKNNS |
| 2193 | AQLAGAVSR |
| 2194 | AQGARMVRE |
| 2195 | AQGTRLPAM |
| 2196 | AQGVHGYSA |
| 2197 | AQYPAATAI |
| 2198 | AQGMRAATS |
| 2199 | AQHRPPGAP |
| 2200 | AQQSTRPAV |
| 2201 | AQQDHGTKP |
| 2202 | AQPIPGKSE |
| 2203 | AQPTLPNRG |
| 2204 | AQVWSDRGM |
| 2205 | AQLAVSYKN |
| 2206 | AQGSLRRGM |
| 2207 | AQALNNQHR |
| 2208 | AQQRHSYDS |
| 2209 | AQGNVTKFF |
| 2210 | AQKFAPSTT |
| 2211 | AQMPSNSPN |
| 2212 | AQRLHPLDA |
| 2213 | AQMPRITTS |
| 2214 | AQMLGRSTP |
| 2215 | AQLREKSYA |
| 2216 | AQMTVRLQP |
| 2217 | AQTSPVKMR |
| 2218 | AQNSSGDRS |
| 2219 | AQVTSTAVT |
| 2220 | AQISMSRSD |
| 2221 | AQLGTTNMP |
| 2222 | AQTSDREKA |
| 2223 | AQPRTVPMP |
| 2224 | AQSTYHTVN |
| 2225 | AQPSALGGK |
| 2226 | AQKANTTTV |
| 2227 | AQVISPVRP |
| 2228 | AQPAKYPET |
| 2229 | AQQRDTDRL |
| 2230 | AQGGDQKRT |
| 2231 | AQYPNRPDL |
| 2232 | AQVAHEHRS |
| 2233 | AQCPAARAC |
| 2234 | AQKPGPAMV |
| 2235 | AQNDRANRV |
| 2236 | AQDREWKFG |
| 2237 | AQAFNRAAT |
| 2238 | AQVASGKVL |
| 2239 | AQTSRSSQI |
| 2240 | AQLKPQTPP |
| 2241 | AQNALVNQF |
| 2242 | AQLSHTITA |
| 2243 | AQNISGRPI |
| 2244 | AQTTGHTVH |
| 2245 | AQAPFQVGN |
| 2246 | AQVGPRNGN |
| 2247 | AQMGQKHVA |
| 2248 | AQVPTNRTG |
| 2249 | AQPDTSRPL |
| 2250 | AQADLGRGL |
| 2251 | AQNRTERSY |
| 2252 | AQTYTLGEP |
| 2253 | AQFTMTFPP |
| 2254 | AQTYSRPQN |
| 2255 | AQLTGHHNP |
| 2256 | AQVTQDQSS |
| 2257 | AQHQQKSLQ |
| 2258 | AQTRNLDVA |
| 2259 | AQLLTSSFG |
| 2260 | AQFSNRSAS |
| 2261 | AQNNYGRSN |
| 2262 | AQGSYPNCQ |
| 2263 | AQSGPKAPG |
| 2264 | AQNSIMAPP |
| 2265 | AQMLGVRSS |
| 2266 | AQGRYVVEQ |
| 2267 | AQPSQVSAS |
| 2268 | AQPRLENSV |
| 2269 | AQLYPPNMP |
| 2270 | AQRSQIPSV |
| 2271 | AQKALSGPM |
| 2272 | AQASQPAAR |
| 2273 | AQSRPTSAP |
| 2274 | AQNGPRYSS |
| 2275 | AQKTGTSIQ |
| 2276 | AQHQVYEHS |
| 2277 | AQTVVRNVS |
| 2278 | AQCMAAQCR |
| 2279 | AQWNYMNRA |
| 2280 | AQMLPSGNP |
| 2281 | AQETPAVRM |
| 2282 | AQAKEKPEK |
| 2283 | AQHGGGSRY |
| 2284 | AQLNDATRY |
| 2285 | AQESSYSYK |
| 2286 | AQTVIAFGD |
| 2287 | AQTVRTGIS |
| 2288 | AQTIMSHNV |
| 2289 | AQNMSLAFK |
| 2290 | AQTLNFQSS |
| 2291 | AQNSSITYL |
| 2292 | AQSMTAKNL |
| 2293 | AQIGEYRLK |
| 2294 | AQSMTQYGL |
| 2295 | AQNQNPVRT |
| 2296 | AQQSPIMRM |
| 2297 | AQSAVRMTG |
| 2298 | AQTILQLGE |
| 2299 | AQRETNAFI |
| 2300 | AQTVTMQYA |
| 2301 | AQTVMSQVS |
| 2302 | AQHPLLTFG |
| 2303 | AQAYDPNAR |
| 2304 | AQENDIRRY |
| 2305 | AQSIGVYGL |
| 2306 | AQNVLKNGS |
| 2307 | AQDPVKLGR |
| 2308 | AQEVPVYRK |
| 2309 | AQEVVMRTS |
| 2310 | AQVQFNRDF |
| 2311 | AQTQSPLPM |
| 2312 | AQNITKLGA |
| 2313 | AQSPFQNGL |
| 2314 | AQNPVKMGA |
| 2315 | AQVATAQLN |
| 2316 | AQQRHAYDS |
| 2317 | AQTVQHQSS |
| 2318 | AQFQSPVTV |
| 2319 | AQTTTSQRE |
| 2320 | AQHVVKMSG |
| 2321 | AQTMVGPGV |
| 2322 | AQNTLSQMS |
| 2323 | AQYVTMQVQ |
| 2324 | AQMSNLVVS |
| 2325 | AQSMVAGGG |
| 2326 | AQVDVWGRA |
| 2327 | AQAMVKAGA |
| 2328 | AQTVLPVSR |
| 2329 | AQLSLKYRE |
| 2330 | AQQWELTPM |
| 2331 | AQQMVRGGE |
| 2332 | AQSPFKGGV |
| 2333 | AQNVVGLGK |
| 2334 | AQQSPVKMD |
| 2335 | AQGVIREGN |
| 2336 | AQTTTNQIK |
| 2337 | AQVISYQIN |
| 2338 | AQRVTQSGE |
| 2339 | AQSMIKLGL |
| 2340 | AQTVSYQVN |
| 2341 | AQLKLSSKG |
| 2342 | AQAMTALGA |
| 2343 | AQSPVKTGL |
| 2344 | AQRSSLSSL |
| 2345 | AQSMAQNNK |
| 2346 | AQGQSPLPH |
| 2347 | AQHSVMTYV |
| 2348 | AQLSDMKRY |
| 2349 | AQGGLPKYG |
| 2350 | AQSMFSQVE |
| 2351 | AQSSRVSSP |
| 2352 | AQQVVKMGL |
| 2353 | AQHPVGLGM |
| 2354 | AQTAQTRLM |
| 2355 | AQTVVKMGT |
| 2356 | AQSIGTYQV |
| 2357 | AQGVLKMSS |
| 2358 | AQQAPIRLG |
| 2359 | AQPRNDPRY |
| 2360 | AQSVIKSGE |
| 2361 | AQKWDNSAS |
| 2362 | AQMPTGMGL |
| 2363 | AQTPLALGE |
| 2364 | AQMVVRSGE |
| 2365 | AQQVVKSGM |
| 2366 | AQKDGVSKQ |
| 2367 | AQAWTGAPK |
| 2368 | AQLNTKFDS |
| 2369 | AQSPVKFGT |
| 2370 | AQSVYKTGE |
| 2371 | AQQRSPPAK |
| 2372 | AQPSPAGRT |
| 2373 | AQNAVRMGE |
| 2374 | AQLLLRWVR |
| 2375 | AQRGNINPP |
| 2376 | AQYSKLVTP |
| 2377 | AQDKQAGGR |
| 2378 | AQTVLRQGE |
| 2379 | AQRVTGMGH |
| 2380 | AQGVTQMSH |
| 2381 | AQGPVGMTG |
| 2382 | AQLIGRYEL |
| 2383 | AQQSRSGSG |
| 2384 | AQNASSPRG |
| 2385 | AQSVLKNGA |
| 2386 | AQSPIKLSH |
| 2387 | AQTILKLGS |
| 2388 | AQSVSAQRS |
| 2389 | AQSFHPPVS |
| 2390 | AQTVLRTGQ |
| 2391 | AQGAISHKT |
| 2392 | AQRPMTSDY |
| 2393 | AQSIHYQKE |
| 2394 | AQEVLRPRP |
| 2395 | AQQMIKVSN |
| 2396 | AQEVRMQKS |
| 2397 | AQTLKIQIE |
| 2398 | AQLVRVTSP |
| 2399 | AQAVIRIGA |
| 2400 | AQSGVSRVG |
| 2401 | AQSPIKSHG |
| 2402 | AQTVQRQTS |
| 2403 | AQPMPRSPG |
| 2404 | AQSSNSPFS |
| 2405 | AQGPLREGM |
| 2406 | AQSVLHQIH |
| 2407 | AQRTGWDEG |
| 2408 | AQMNSLSYL |
| 2409 | AQGVQVGRL |
| 2410 | AQAVTPRNI |
| 2411 | AQNTVMQTS |
| 2412 | AQSLVRMGE |
| 2413 | AQGSSLVRP |
| 2414 | AQTTAKPSL |
| 2415 | AQYVNSGAL |
| 2416 | AQTMFKNGE |
| 2417 | AQQNATARV |
| 2418 | AQSVISLGE |
| 2419 | AQSPLARGE |
| 2420 | AQYTTQQTS |
| 2421 | AQGPVIQAR |
| 2422 | AQATLAKSA |
| 2423 | AQGCSKKTG |
| 2424 | AQTVYSGIS |
| 2425 | AQFVAAKTQ |
| 2426 | AQTSSQRAG |
| 2427 | AQSVLSHNI |
| 2428 | AQGKGLSTI |
| 2429 | AQRSSSASA |
| 2430 | AQGMSLVSR |
| 2431 | AQTTYTQVT |
| 2432 | AQTLNKQIT |
| 2433 | AQYLNSPMS |
| 2434 | AQSGGCAGC |
| 2435 | AQTAKYTTN |
| 2436 | AQFINSSGL |
| 2437 | AQASTPYSV |
| 2438 | AQNVIKNGS |
| 2439 | AQLMPSPLR |
| 2440 | AQMRSSHSP |
| 2441 | AQWKLYAGD |
| 2442 | AQTTTLQVD |
| 2443 | AQTVMVQRS |
| 2444 | AQYLNGPVL |
| 2445 | AQGPPATTP |
| 2446 | AQRVHSQGP |
| 2447 | AQTTYNQMD |
| 2448 | AQHKSSTAG |
| 2449 | AQMTGLKFS |
| 2450 | AQGVVRQTS |
| 2451 | AQVTGRASP |
| 2452 | AQAIEPKRN |
| 2453 | AQSINYQVT |
| 2454 | AQNIRVQID |
| 2455 | AQRVVKQGE |
| 2456 | AQWRPTNAN |
| 2457 | AQVLVGGRG |
| 2458 | AQTIRTGIA |
| 2459 | AQTPIRMTH |
| 2460 | AQKVTGMGI |
| 2461 | AQTVVKHGL |
| 2462 | AQTVIAFGA |
| 2463 | AQRNGQSGL |
| 2464 | AQTEKMGLR |
| 2465 | AQIREHSKT |
| 2466 | AQWNNARSA |
| 2467 | AQMYGLMEE |
| 2468 | AQPFSPIFV |
| 2469 | AQTSELPVV |
| 2470 | AQSVMHQAS |
| 2471 | AQTVNYQIA |
| 2472 | AQMRPLLKE |
| 2473 | AQVVGTNTG |
| 2474 | AQLLSLSSK |
| 2475 | AQLTTHQFS |
| 2476 | AQSMPSPNR |
| 2477 | AQHRTCVAF |
| 2478 | AQRITMMGE |
| 2479 | AQQDPVRLK |
| 2480 | AQGVVAYSS |
| 2481 | AQTRMQNNT |
| 2482 | AQGVVGMKG |
| 2483 | AQGPVQISG |
| 2484 | AQTTVQQMY |
| 2485 | AQSPLRAGA |
| 2486 | AQSQSPVRL |
| 2487 | AQKSVSRDG |
| 2488 | AQSMLGAHP |
| 2489 | AQLLGNNII |
| 2490 | AQKMGHGMG |
| 2491 | AQLRSVPQA |
| 2492 | AQFGEVNNP |
| 2493 | AQNVIKTGS |
| 2494 | AQQLRGSQG |
| 2495 | AQDLSGCSK |
| 2496 | AQVQTPIRV |
| 2497 | AQVGGVIKG |
| 2498 | AQUATHNN |
| 2499 | AQLEYARSL |
| 2500 | AQLTYYGIE |
| 2501 | AQVLTSHNT |
| 2502 | AQYVNSGPA |
| 2503 | AQGVVAMSS |
| 2504 | AQFGESEGV |
| 2505 | AQRLNSGVP |
| 2506 | AQVSSTSVP |
| 2507 | AQAAINRRG |
| 2508 | AQKVDSRVF |
| 2509 | AQVMPKSQF |
| 2510 | AQLLALCDK |
| 2511 | AQGVIGMLG |
| 2512 | AQGDTARVW |
| 2513 | AQIMIRMGE |
| 2514 | AQSVLLQFS |
| 2515 | AQPVKIYNE |
| 2516 | AQATVSKSR |
| 2517 | AQSNLHGGG |
| 2518 | AQNTTKLSF |
| 2519 | AQKGDYSKG |
| 2520 | AQNIVRLGL |
| 2521 | AQWQLGASV |
| 2522 | AQMACRLVL |
| 2523 | AQTTLAQYD |
| 2524 | AQGRLSEVK |
| 2525 | AQLTQRIGS |
| 2526 | AQPFKEVSR |
| 2527 | AQAFDVNGR |
| 2528 | AQHTANQKS |
| 2529 | AQQQAPMRS |
| 2530 | AQTVIVQRS |
| 2531 | AQNPTSKPN |
| 2532 | AQRNKLSSD |
| 2533 | AQRVSTGIS |
| 2534 | AQPTKYGQN |
| 2535 | AQVVGHTKL |
| 2536 | AQGWADHTA |
| 2537 | AQMEDPNQY |
| 2538 | AQIMFDVPY |
| 2539 | AQLREWKES |
| 2540 | AQNQSLVYK |
| 2541 | AQNPYGPGT |
| 2542 | AQKFREGDI |
| 2543 | AQYNKLQAG |
| 2544 | AQFINGPEH |
| 2545 | AQHVRAGEN |
| 2546 | AQVLRSQID |
| 2547 | AQVKGLEKG |
| 2548 | AQVCLVARV |
| 2549 | AQRRVLETP |
| 2550 | AQGNGSTKF |
| 2551 | AQSVSLGTD |
| 2552 | AQGVVAMMH |
| 2553 | AQHHPLSSI |
| 2554 | AQNPVGGRG |
| 2555 | AQSVTPVGK |
| 2556 | AQTMLKAGE |
| 2557 | AQSPVRGGE |
| 2558 | AQTQRLGIA |
| 2559 | AQMITRQID |
| 2560 | AQGRTPPSQ |
| 2561 | AQSMNQNRR |
| 2562 | AQLNTRVVS |
| 2563 | AQKQSASSG |
| 2564 | AQVRTGGAA |
| 2565 | AQSLYSQLD |
| 2566 | AQGVVQQQS |
| 2567 | AQNQTPIRS |
| 2568 | AQTAVSQVR |
| 2569 | AQASEYRAL |
| 2570 | AQGARTQDR |
| 2571 | AQRGSSMRD |
| 2572 | AQQTPVARM |
| 2573 | AQGASMSYK |
| 2574 | AQVTRAVTG |
| 2575 | AQCMSICSA |
| 2576 | AQGNVTKSA |
| 2577 | AQRGATSTN |
| 2578 | AQVYARSLP |
| 2579 | AQTKIPNGQ |
| 2580 | AQCMASKSC |
| 2581 | AQNPVKIGF |
| 2582 | AQLDKMEST |
| 2583 | AQYINGPSM |
| 2584 | AQDNITSFS |
| 2585 | AQYKVTSGN |
| 2586 | AQLSSSLKT |
| 2587 | AQNPVGPRG |
| 2588 | AQQFSKSTV |
| 2589 | AQVRTNPLK |
| 2590 | AQTQSTRMG |
| 2591 | AQGKSLVFT |
| 2592 | AQMMTGHGL |
| 2593 | AQGAGYSSI |
| 2594 | AQIGTTYKS |
| 2595 | AQSTLNQRS |
| 2596 | AQRGVLPHL |
| 2597 | AQRSTGDRF |
| 2598 | AQQRSSLVQ |
| 2599 | AQWSTSCAQ |
| 2600 | AQTSLRSVG |
| 2601 | AQGPTKFGG |
| 2602 | AQGVIAFGG |
| 2603 | AQRPQEHKM |
| 2604 | AQAQISGKI |
| 2605 | AQGDEALAT |
| 2606 | AQMDGWGRT |
| 2607 | AQAVIKFNG |
| 2608 | AQNARTHNV |
| 2609 | AQPFPARQT |
| 2610 | AQFPSGTKN |
| 2611 | AQDIRVSTG |
| 2612 | AQAVVKISH |
| 2613 | AQGLSLSSK |
| 2614 | AQEKAQVGR |
| 2615 | AQENSISFR |
| 2616 | AQNEPFMMD |
| 2617 | AQYVNSAGF |
| 2618 | AQNRGLNGM |
| 2619 | AQSHAVKYP |
| 2620 | AQLSAFTNM |
| 2621 | AQSFDPNGS |
| 2622 | AQVNERARL |
| 2623 | AQSNSYSYK |
| 2624 | AQRFSEQIS |
| 2625 | AQSGCDSLS |
| 2626 | AQTQNPVDN |
| 2627 | AQSPIPAGA |
| 2628 | AQSMVGPGN |
| 2629 | AQTMTKRGE |
| 2630 | AQPTDVRRM |
| 2631 | AQNIYKSAS |
| 2632 | AQGMSLTFR |
| 2633 | AQSPIGIHG |
| 2634 | AQSYSKLTI |
| 2635 | AQSMTGIGM |
| 2636 | AQTPYQTGN |
| 2637 | AQLTRPGTA |
| 2638 | AQFVMYAYD |
| 2639 | AQADGLFKS |
| 2640 | AQRKLRLQD |
| 2641 | AQHGNAGYA |
| 2642 | AQPSSLSSR |
| 2643 | AQSLIRQAS |
| 2644 | AQLLRSSAG |
| 2645 | AQSVTGTGF |
| 2646 | AQEKGTHFL |
| 2647 | AQGMRSTSR |
| 2648 | AQDVVGLGL |
| 2649 | AQGVVALSS |
| 2650 | AQMNKVSQG |
| 2651 | AQYSGSFMS |
| 2652 | AQGTRLPHI |
| 2653 | AQVFATHNV |
| 2654 | AQGVVGNSS |
| 2655 | AQSSSYYDA |
| 2656 | AQLHARASS |
| 2657 | AQMTVSQKS |
| 2658 | AQWAGVTIS |
| 2659 | AQIYKTTST |
| 2660 | AQHDGVSRT |
| 2661 | AQWHTQCSA |
| 2662 | AQRTPSHAN |
| 2663 | AQSTPRSHG |
| 2664 | AQRAADPTV |
| 2665 | AQILTYAYD |
| 2666 | AQKTVPLWP |
| 2667 | AQIVRPPLT |
| 2668 | AQQMIRLGD |
| 2669 | AQSMTPNGG |
| 2670 | AQGVVQLGW |
| 2671 | AQSQCTNRL |
| 2672 | AQGSNSKVS |
| 2673 | AQFTNKPPV |
| 2674 | AQAISAYSC |
| 2675 | AQGLPCVAS |
| 2676 | AQDNSRVPR |
| 2677 | AQTYRSGSA |
| 2678 | AQMRERKMF |
| 2679 | AQTVTRQTT |
| 2680 | AQGVVREGA |
| 2681 | AQTITIPLY |
| 2682 | AQYLNSSGS |
| 2683 | AQASMPSAR |
| 2684 | AQAGRENES |
| 2685 | AQGKITPMY |
| 2686 | AQSYETDAL |
| 2687 | AQQPGRGHL |
| 2688 | AQYLKDRTM |
| 2689 | AQLKSLSYL |
| 2690 | AQKRYGHRD |
| 2691 | AQRHEVRRN |
| 2692 | AQDSKAVST |
| 2693 | AQGVIGNGG |
| 2694 | AQLKGISKG |
| 2695 | AQSGFTTVN |
| 2696 | AQSAPPIPR |
| 2697 | AQTGKMQCS |
| 2698 | AQGFVPTLT |
| 2699 | AQKTVLIST |
| 2700 | AQWSSIVTS |
| 2701 | AQIYALSSG |
| 2702 | AQSVVYQLT |
| 2703 | AQAIIRMGE |
| 2704 | AQITTSQLT |
| 2705 | AQWMKATTV |
| 2706 | AQVSGSTLR |
| 2707 | AQTRAAVCS |
| 2708 | AQRDAKLMD |
| 2709 | AQKSGLDKF |
| 2710 | AQKWDKLSS |
| 2711 | AQNPVRSGV |
| 2712 | AQSPLPIVR |
| 2713 | AQGPVVKDR |
| 2714 | AQSMTGVNP |
| 2715 | AQTPTARLS |
| 2716 | AQARSVPGS |
| 2717 | AQSTLNQVS |
| 2718 | AQMKHAVNA |
| 2719 | AQSMTPSGS |
| 2720 | AQPPVVQSG |
| 2721 | AQSTMNQYE |
| 2722 | AQYLMKCAS |
| 2723 | AQGIASSRT |
| 2724 | AQNESYAYK |
| 2725 | AQNPVKMSQ |
| 2726 | AQDVIRLGK |
| 2727 | AQSMIRNGA |
| 2728 | AQLVFGRDM |
| 2729 | AQLWETTLP |
| 2730 | AQSVDGLKS |
| 2731 | AQSVSFQIT |
| 2732 | AQGMSLLTR |
| 2733 | AQGAHGTPF |
| 2734 | AQDPLLYVK |
| 2735 | AQHVRQTSS |
| 2736 | AQRTGSGVH |
| 2737 | AQLVYARTM |
| 2738 | AQRKKTPRR |
| 2739 | AQVVITPAR |
| 2740 | AQA VIRNGS |
| 2741 | AQVPNAFSK |
| 2742 | AQLQMERSK |
| 2743 | AQRVKVSGD |
| 2744 | AQGQLACVT |
| 2745 | AQKLKKDYE |
| 2746 | AQTYIGPGE |
| 2747 | AQNRADKTI |
| 2748 | AQQETFAWR |
| 2749 | AQATKTRIT |
| 2750 | AQMLIPVSC |
| 2751 | AQTHSAMSS |
| 2752 | AQQNPVRAE |
| 2753 | AQTGPMKME |
| 2754 | AQMVFTSAT |
| 2755 | AQYFNEQRS |
| 2756 | AQNPVGAGG |
| 2757 | AQGVVAFKS |
| 2758 | AQNVLYQTS |
| 2759 | AQDNLHTAR |
| 2760 | AQSQGLVKM |
| 2761 | AQTGLKGNG |
| 2762 | AQYLVSNLS |
| 2763 | AQLESYRTA |
| 2764 | AQVPRQVNF |
| 2765 | AQSVTFQLS |
| 2766 | AQLNILMEV |
| 2767 | AQSGIHNQT |
| 2768 | AQQLFACNK |
| 2769 | AQGTIAKWT |
| 2770 | AQNLLRAKL |
| 2771 | AQNRTNAYR |
| 2772 | AQEQMSFHG |
| 2773 | AQIGAHEFS |
| 2774 | AQTLNTHNT |
| 2775 | AQSVVGNGV |
| 2776 | AQSLNMMGV |
| 2777 | AQTKMRDGG |
| 2778 | AQTNIEATP |
| 2779 | AQRTLPDTK |
| 2780 | AQSVVRSGG |
| 2781 | AQICTSTTY |
| 2782 | AQTCEEIVL |
| 2783 | AQGTPIRMT |
| 2784 | AQGTYCAGQ |
| 2785 | AQACGLSDV |
| 2786 | AQERLEKRN |
| 2787 | AQWCERYRE |
| 2788 | AQSKSDAPR |
| 2789 | AQVLSGHHG |
| 2790 | AQDLLKICR |
| 2791 | AQFLDLGQM |
| 2792 | AQEVYKPYH |
| 2793 | AQQGVSGKH |
| 2794 | AQPIGGFME |
| 2795 | AQTNTRLTR |
| 2796 | AQHRLTAAH |
| 2797 | AQAYLDRNT |
| 2798 | AQYRNAMLG |
| 2799 | AQSPKLFPA |
| 2800 | AQNMTKMSA |
| 2801 | AQVKGNFIA |
| 2802 | AQSPVSMGY |
| 2803 | AQLSVTGRT |
| 2804 | AQFVDPNQR |
| 2805 | AQPLVERIV |
| 2806 | AQFVNCLED |
| 2807 | AQATRGNLL |
| 2808 | AQGYMLPHV |
| 2809 | AQIMYNRDF |
| 2810 | AQIIGANCP |
| 2811 | AQKLVGRLQ |
| 2812 | AQFNRPGTN |
| 2813 | AQKGTERKL |
| 2814 | AQRGNMCRA |
| 2815 | AQANRMKCT |
| 2816 | AQRWDLETA |
| 2817 | AQQKGQGFQ |
| 2818 | AQCVSMKRD |
| 2819 | AQERTGTLL |
| 2820 | AQTARSGIY |
| 2821 | AQYRSMQVD |
| 2822 | AQTVLVQVM |
| 2823 | AQSWSSQVD |
| 2824 | AQQYDVNGR |
| 2825 | AQVVTSQKS |
| 2826 | AQSWDNQVT |
| 2827 | AQTQNSARS |
| 2828 | AQNHQSGRV |
| 2829 | AQSNKSPNH |
| 2830 | AQVNGLILR |
| 2831 | AQTKYHLDL |
| 2832 | AQCVVKCGV |
| 2833 | AQQMTQGGS |
| 2834 | AQRVLQEHN |
| 2835 | AQASSNASK |
| 2836 | AQKNSLSTL |
| 2837 | AQFRSQSLS |
| 2838 | AQICEQFVY |
| 2839 | AQESDVSQY |
| 2840 | AQVMREIPI |
| 2841 | AQLSGRTYN |
| 2842 | AQAEAPRGF |
| 2843 | AQQNPVRAL |
| 2844 | AQKLDKLIS |
| 2845 | AQPRTGHTT |
| 2846 | AQISVAERM |
| 2847 | AQSRPAPLH |
| 2848 | AQGTTILRP |
| 2849 | AQLKHKMYY |
| 2850 | AQLDTLYLN |
| 2851 | AQFRNNTYR |
| 2852 | AQNRFEPRR |
| 2853 | AQFYTSHNT |
| 2854 | AQQTRKSAA |
| 2855 | AQTLHSQYS |
| 2856 | AQLMARWPE |
| 2857 | AQRALKHHN |
| 2858 | AQNPHSQTV |
| 2859 | AQAVIRDAG |
| 2860 | AQLAASQPS |
| 2861 | AQRLECRMI |
| 2862 | AQYPSAVCK |
| 2863 | AQKDGHNKG |
| 2864 | AQKGAVTVN |
| 2865 | AQECIGNKD |
| 2866 | AQTSRPVST |
| 2867 | AQNGGFSSK |
| 2868 | AQTARPLAS |
| 2869 | AQIKGGGSK |
| 2870 | AQSENSQVF |
| 2871 | AQKGMPKDR |
| 2872 | AQTRQIPAI |
| 2873 | AQTRTNQGP |
| 2874 | AQRSLNHGG |
| 2875 | AQKSWEDKC |
| 2876 | AQNMLRMGA |
| 2877 | AQQCGKPSS |
| 2878 | AQCVPHTST |
| 2879 | AQHRLWDEE |
| 2880 | AQQAPRISA |
| 2881 | AQSWDKLDK |
| 2882 | AQERKSAVS |
| 2883 | AQAANPRRS |
| 2884 | AQAGGLTKY |
| 2885 | AQSAIRMKC |
| 2886 | AQSRVKTHV |
| 2887 | AQVTTKQIH |
| 2888 | AQLNRGGAA |
| 2889 | AQRSQAMVS |
| 2890 | AQIRWVRGV |
| 2891 | AQSDCGTLF |
| 2892 | AQQNRFPDQ |
| 2893 | AQGNPLRTL |
| 2894 | AQAYGVSSP |
| 2895 | AQSGNRMTC |
| 2896 | AQNTYGIDM |
| 2897 | AQPIPKPNL |
| 2898 | AQAVANRPM |
| 2899 | AQTFLQMGS |
| 2900 | AQMMTGMGL |
| 2901 | AQRYGNGCV |
| 2902 | AQSPLFSSP |
| 2903 | AQGIGGNAR |
| 2904 | AQGGIRLVR |
| 2905 | AQNCEVSTY |
| 2906 | AQKSVRLSY |
| 2907 | AQKTDLFAI |
| 2908 | AQNPVGARG |
| 2909 | AQTLIRLPL |
| 2910 | AQNTYFVNV |
| 2911 | AQSVPWGDD |
| 2912 | AQKRRTLNM |
| 2913 | AQYACELVY |
| 2914 | AQLIPLTRA |
| 2915 | AQNHYSRLE |
| 2916 | AQKMHGLGE |
| 2917 | AQFIVSHNV |
| 2918 | AQSVRMIQT |
| 2919 | AQSIVPMGA |
| 2920 | AQRELSIKD |
| 2921 | AQNVTPINK |
| 2922 | AQYQSPLRE |
| 2923 | AQGKDRSLP |
| 2924 | AQRTCDMIS |
| 2925 | AQSQIKWGE |
| 2926 | AQFVSVQVS |
| 2927 | AQFPNPSGR |
| 2928 | AQGPSISYK |
| 2929 | AQAVAKGFC |
| 2930 | AQQNPVLNQ |
| 2931 | AQGSKTQLR |
| 2932 | AQWLKLCPS |
| 2933 | AQNASKSRF |
| 2934 | AQRKAQAQT |
| 2935 | AQTRYAQIP |
| 2936 | AQRVVTSIS |
| 2937 | AQKNLKHVW |
| 2938 | AQTPYIFAN |
| 2939 | AQSDSCFGS |
| 2940 | AQPGTRASG |
| 2941 | AQGRLNATT |
| 2942 | AQGIIRMDT |
| 2943 | AQERGMAKP |
| 2944 | AQLVGIKQI |
| 2945 | AQYKATLRL |
| 2946 | AQAVMKRIK |
| 2947 | AQLNDIVRF |
| 2948 | AQKLSLSYC |
| 2949 | AQSYLAMNP |
| 2950 | AQRIQHLSG |
| 2951 | AQDNVCDVV |
| 2952 | AQPQNGINS |
| 2953 | AQKADGVRE |
| 2954 | AQCHDLPRI |
| 2955 | AQCRVGDMK |
| 2956 | AQLIGRSSS |
| 2957 | AQDGHLDVV |
| 2958 | AQNSKTLSA |
| 2959 | AQTKWHGDE |
| 2960 | AQRRMHDRL |
| 2961 | AQNRDINQH |
| 2962 | AQPYDPNRN |
| 2963 | AQSTKRPYD |
| 2964 | AQKRGMDKP |
| 2965 | AQGKEVAWQ |
| 2966 | AQGRYVPGD |
| 2967 | AQGVVMLGG |
| 2968 | AQDNYRTKG |
| 2969 | AQTVISQRS |
| 2970 | AQLSRSPSL |
| 2971 | AQAMHAGGE |
| 2972 | AQSPLRTGA |
| 2973 | AQLISNSIL |
| 2974 | AQTVLKQIE |
| 2975 | AQVVFSQTS |
| 2976 | AQTMLKLGD |
| 2977 | AQPPSPRRP |
| 2978 | AQVRPGISI |
| 2979 | AQGKYMPSN |
| 2980 | AQGILAMNG |
| 2981 | AQSPYRNHS |
| 2982 | AQVNRDTKP |
| 2983 | AQTMVGNGM |
| 2984 | AQSPFAMGT |
| 2985 | AQKAGVMKA |
| 2986 | AQTSYTGIS |
| 2987 | AQVQSPVKN |
| 2988 | AQGVVALLS |
| 2989 | AQGKSLLYT |
| 2990 | AQLHLGKML |
| 2991 | AQARSESRT |
| 2992 | AQGSGINTM |
| 2993 | AQTLPKIGL |
| 2994 | AQSANTGIW |
| 2995 | AQTPIKGGI |
| 2996 | AQKHGVASG |
| 2997 | AQARGLIKA |
| 2998 | AQTTCLMTF |
| 2999 | AQHNSVSYL |
| 3000 | AQTVLNQND |
| 3001 | AQTIMVQMT |
| 3002 | AQFLVNIAG |
| 3003 | AQELCGTRE |
| 3004 | AQKIVNDLP |
| 3005 | AQPFDRNGS |
| 3006 | AQVSIRSHC |
| 3007 | AQDNPRFYL |
| 3008 | AQKLGQDKH |
| 3009 | AQMAACHTD |
| 3010 | AQRMSSVNG |
| 3011 | AQLHKPAER |
| 3012 | AQTIVPTLK |
| 3013 | AQTPIRSAS |
| 3014 | AQSQTRTHL |
| 3015 | AQLPLKSAN |
| 3016 | AQEVRKVYS |
| 3017 | AQRQLVCAH |
| 3018 | AQTGQFKRD |
| 3019 | AQKDNRKSN |
| 3020 | AQGPIGIGG |
| 3021 | AQRSIPSAC |
| 3022 | AQWDCDVSK |
| 3023 | AQTPVSNRQ |
| 3024 | AQWKGGPTS |
| 3025 | AQSCTTVGF |
| 3026 | AQYVPLMHC |
| 3027 | AQNLSSPRA |
| 3028 | AQNQLIRHP |
| 3029 | AQWINSGPS |
| 3030 | AQSIMGLGL |
| 3031 | AQTTVMQVL |
| 3032 | AQTSFSGRG |
| 3033 | AQLSSRSSP |
| 3034 | AQRSAHLTR |
| 3035 | AQKPGMLKV |
| 3036 | AQNPRTRRD |
| 3037 | AQVNQSRAY |
| 3038 | AQMPIGPRP |
| 3039 | AQVVITTGM |
| 3040 | AQTTMKQVT |
| 3041 | AQISLAQRI |
| 3042 | AQKPIGMGN |
| 3043 | AQLYVTSCS |
| 3044 | AQGFQKWKD |
| 3045 | AQSQWAGPT |
| 3046 | AQWASKSGG |
| 3047 | AQAQYKAQD |
| 3048 | AQSPVKSGH |
| 3049 | AQKMSGVGE |
| 3050 | AQEGSGWRE |
| 3051 | AQAMTGMGA |
| 3052 | AQHPVGMGA |
| 3053 | AQTASFQTS |
| 3054 | AQVTSRNII |
| 3055 | AQGGGLTYV |
| 3056 | AQHRFMRCA |
| 3057 | AQGIVRLGE |
| 3058 | AQTRKCMYG |
| 3059 | AQPKARLLD |
| 3060 | AQGRYTGTE |
| 3061 | AQDQSLVYK |
| 3062 | AQCVYTLAV |
| 3063 | AQLEVIYLD |
| 3064 | AQIADKGMR |
| 3065 | AQLTDMLIR |
| 3066 | AQQLKGLAE |
| 3067 | AQSPKGIYF |
| 3068 | AQLMVAMGM |
| 3069 | AQVKFIEPY |
| 3070 | AQRWLAEEE |
| 3071 | AQFLTRRYK |
| 3072 | AQNLVGARG |
| 3073 | AQVYASHNY |
| 3074 | AQRFNDDRR |
| 3075 | AQHSMASCT |
| 3076 | AQVDWIEDG |
| 3077 | AQSALSAMH |
| 3078 | AQVIVMDIC |
| 3079 | AQMTKPVSS |
| 3080 | AQKISKPMR |
| 3081 | AQRVGRTEG |
| 3082 | AQQNSLTFR |
| 3083 | AQLETCLRL |
| 3084 | AQHRSVVGT |
| 3085 | AQNPFQNGL |
| 3086 | AQVRAARSG |
| 3087 | AQTVKNQVN |
| 3088 | AQQAAKVDR |
| 3089 | AQWLKYTPE |
| 3090 | AQIPSLRAM |
| 3091 | AQDGREKFG |
| 3092 | AQLYTLHDM |
| 3093 | AQKFGMRSS |
| 3094 | AQIKQNTYS |
| 3095 | AQCSYGTNH |
| 3096 | AQDLTRSCS |
| 3097 | AQTATMQMS |
| 3098 | AQEVSGLHI |
| 3099 | AQMCGNESK |
| 3100 | AQSVRQERN |
| 3101 | AQKSCSSIV |
| 3102 | AQLRTSTPL |
| 3103 | AQDKLQTVR |
| 3104 | AQRMSETRN |
| 3105 | AQFVRYAHD |
| 3106 | AQGKYSGIE |
| 3107 | AQMPVGMGE |
| 3108 | AQVSDMVRY |
| 3109 | AQTVNVQIH |
| 3110 | AQRLWKDEA |
| 3111 | AQDSEVGAT |
| 3112 | AQDPCSVAH |
| 3113 | AQSNTVTNR |
| 3114 | AQNRDVRLS |
| 3115 | AQKREYGEV |
| 3116 | AQLSCGGSK |
| 3117 | AQVCLPAPL |
| 3118 | AQKCIPLSR |
| 3119 | AQNEISIKA |
| 3120 | AQRNQNLDS |
| 3121 | AQEVVAKKS |
| 3122 | AQLPVKLGM |
| 3123 | AQETVNCQS |
| 3124 | AQHRSTYRV |
| 3125 | AQRSSLSTL |
| 3126 | AQLSDVKRH |
| 3127 | AQVVSYCRP |
| 3128 | AQSALNNSM |
| 3129 | AQAKPLKSM |
| 3130 | AQSVSNQID |
| 3131 | AQSTLNQNS |
| 3132 | AQSPLRTGE |
| 3133 | AQKPARAAW |
| 3134 | AQEYGLFSD |
| 3135 | AQDTRSPPR |
| 3136 | AQNMVGSGF |
| 3137 | AQGPTGSRA |
| 3138 | AQTANHGTL |
| 3139 | AQSTRDFAK |
| 3140 | AQSNRSPGG |
| 3141 | AQYMNSSGS |
| 3142 | AQNIPQYAR |
| 3143 | AQSVSSGIS |
| 3144 | AQQSPIGMS |
| 3145 | AQPSSLLAM |
| 3146 | AQWINGPPS |
| 3147 | AQNTTTCRA |
| 3148 | AQVCVPSNR |
| 3149 | AQMKTVRLL |
| 3150 | AQAAPMTVR |
| 3151 | AQWTPTIMK |
| 3152 | AQKNDTCAV |
| 3153 | AQRHSGQLK |
| 3154 | AQTTKSQLS |
| 3155 | AQNADYHCL |
| 3156 | AQGMKTTMS |
| 3157 | AQDNRTARG |
| 3158 | AQVRVCAGV |
| 3159 | AQGSGRPTF |
| 3160 | AQTVLPRLS |
| 3161 | AQRLVGGSP |
| 3162 | AQMHSGHTC |
| 3163 | AQTVMAQVL |
| 3164 | AQMVVKLGC |
| 3165 | AQAGMAMPR |
| 3166 | AQILVDVCT |
| 3167 | AQFEYMMCH |
| 3168 | AQHTDKEML |
| 3169 | AQSREISGC |
| 3170 | AQKLVAKES |
| 3171 | AQNRLGQVK |
| 3172 | AQRLIATSV |
| 3173 | AQVVNYQVS |
| 3174 | AQISSFVRQ |
| 3175 | AQPMCTVMP |
| 3176 | AQSQSNRLC |
| 3177 | AQMNSRFDT |
| 3178 | AQSPIKFGS |
| 3179 | AQQMIRLSE |
| 3180 | AQCRELPPR |
| 3181 | AQAKHDELV |
| 3182 | AQDGSRVKG |
| 3183 | AQHPGAAKA |
| 3184 | AQWRGSAKT |
| 3185 | AQKKDMTCQ |
| 3186 | AQVNNACRI |
| 3187 | AQLPKHMNV |
| 3188 | AQPYRAQSS |
| 3189 | AQDVIRIGA |
| 3190 | AQQIQHTIR |
| 3191 | AQGSLTCGH |
| 3192 | AQSYLVGVG |
| 3193 | AQQLSAKCR |
| 3194 | AQMDESAHC |
| 3195 | AQGVVRENG |
| 3196 | AQLLESVQV |
| 3197 | AQLDALFAR |
| 3198 | AQGSGLHCA |
| 3199 | AQWVSHTVV |
| 3200 | AQAFVKTSA |
| 3201 | AQVGMSGLC |
| 3202 | AQLQIPACG |
| 3203 | AQGLVRMNS |
| 3204 | AQADSLHVY |
| 3205 | AQNVLTLGL |
| 3206 | AQCRFIPKS |
| 3207 | AQGSLNKCP |
| 3208 | AQTYNENSL |
| 3209 | AQSLYVSSK |
| 3210 | AQTTMHSGC |
| 3211 | AQNFQYQVS |
| 3212 | AQPSSVHRL |
| 3213 | AQSKLTTVS |
| 3214 | AQMDNRYDN |
| 3215 | AQSQNPVQF |
| 3216 | AQTTNLLKW |
| 3217 | AQHADKLRQ |
| 3218 | AQITTKMSY |
| 3219 | AQGKTAECA |
| 3220 | AQPWRDNCT |
| 3221 | AQKGYAEMI |
| 3222 | AQYNPVKAG |
| 3223 | AQTSGRPSL |
| 3224 | AQAMSGAYL |
| 3225 | AQNMLGLGQ |
| 3226 | AQSTSTHNY |
| 3227 | AQAPLLPGH |
| 3228 | AQLARARGD |
| 3229 | AQVGFLYSF |
| 3230 | AQLRGSASK |
| 3231 | AQCYGDVDT |
| 3232 | AQNSGRVLC |
| 3233 | AQTTRSLSY |
| 3234 | AQSPTRYGE |
| 3235 | AQYGGTKNC |
| 3236 | AQSLCKTYV |
| 3237 | AQNLKNCTY |
| 3238 | AQTSGRLMS |
| 3239 | AQAMPRVQG |
| 3240 | AQLGPNVKP |
| 3241 | AQRGLANAL |
| 3242 | AQYSLVRIC |
| 3243 | AQIHIDDPY |
| 3244 | AQNSYGRVM |
| 3245 | AQTSKMSAH |
| 3246 | AQTVRAQIT |
| 3247 | AQRENGRNS |
| 3248 | AQVSKVLPC |
| 3249 | AQRPTSLSQ |
| 3250 | AQLCSDGMH |
| 3251 | AQDVVRQGY |
| 3252 | AQWESGTYA |
| 3253 | AQTVKQMGS |
| 3254 | AQMSRATVP |
| 3255 | AQADLGMCQ |
| 3256 | AQQQRGTPM |
| 3257 | AQCRERAGS |
| 3258 | AQRHGSPDK |
| 3259 | AQKEGLNKG |
| 3260 | AQSDTRVPS |
| 3261 | AQVMRCLDR |
| 3262 | AQSPATATD |
| 3263 | AQQSPIAGV |
| 3264 | AQPRAATAH |
| 3265 | AQTSCSSPL |
| 3266 | AQKQSGSGK |
| 3267 | AQRLQSVLG |
| 3268 | AQSPSLPCI |
| 3269 | AQQIASMNA |
| 3270 | AQWLTTHNV |
| 3271 | AQNRLSSIG |
| 3272 | AQRSAERLP |
| 3273 | AQGARGNWV |
| 3274 | AQPAKFAYD |
| 3275 | AQRGEAITK |
| 3276 | AQGGHFLKA |
| 3277 | AQRNFPTAI |
| 3278 | AQIYTPKTA |
| 3279 | AQVAPRDKL |
| 3280 | AQFQNPVDF |
| 3281 | AQSIIAMGL |
| 3282 | AQSVWTGSV |
| 3283 | AQEPCYTNT |
| 3284 | AQCDGRCLG |
| 3285 | AQNPVGARV |
| 3286 | AQSCDLAAY |
| 3287 | AQNFRRPNA |
| 3288 | AQLKLNIKA |
| 3289 | AQGMLECHF |
| 3290 | AQRGACATS |
| 3291 | AQNQSLERV |
| 3292 | AQYSALVAQ |
| 3293 | AQTNSSSTK |
| 3294 | AQESSCTRD |
| 3295 | AQVHTLCAH |
| 3296 | AQSAGCYLM |
| 3297 | AQTRRAGED |
| 3298 | AQRRTSSEC |
| 3299 | AQVRAVDKT |
| 3300 | AQDMASLKC |
| 3301 | AQRVGCTSY |
| 3302 | AQTLSFQNS |
| 3303 | AQMQSPKKQ |
| 3304 | AQTPVHMGS |
| 3305 | AQGNLDCVY |
| 3306 | AQQRNNALS |
| 3307 | AQTQRPSPQ |
| 3308 | AQRAQERRD |
| 3309 | AQQYDPNGT |
| 3310 | AQPRNAGSN |
| 3311 | AQRMSSCTN |
| 3312 | AQDYQRKPT |
| 3313 | AQSLKRQIE |
| 3314 | AQGRYRALE |
| 3315 | AQTRPVTVS |
| 3316 | AQHDMNCTR |
| 3317 | AQTWSTGAS |
| 3318 | AQTVVAQKA |
| 3319 | AQFSSTSGK |
| 3320 | AQGIKFTPN |
| 3321 | AQWSKSERT |
| 3322 | AQDASVALR |
| 3323 | AQDWDHCAE |
| 3324 | AQGGLPDGH |
| 3325 | AQRCGGQHI |
| 3326 | AQRLFATAV |
| 3327 | AQPLECGMV |
| 3328 | AQMYVDWHV |
| 3329 | AQLSSALDD |
| 3330 | AQDDNSERC |
| 3331 | AQRDRCDQP |
| 3332 | AQKSGGCAG |
| 3333 | AQCLKSKSÅ |
| 3334 | AQKISSHNT |
| 3335 | AQSGHLMLT |
| 3336 | AQFEYSPIV |
| 3337 | AQVSTVMGT |
| 3338 | AQRMVVAAA |
| 3339 | AQETADHDW |
| 3340 | AQFTSGRAR |
| 3341 | AQIK GIRCP |
| 3342 | AQPGARPVM |
| 3343 | AQQRYCYGP |
| 3344 | AQSDVECQV |
| 3345 | AQGLRGCGM |
| 3346 | AQQKIGMCT |
| 3347 | AQVPKEIHR |
| 3348 | AQSVSPVDQ |
| 3349 | AQYLASKFR |
| 3350 | AQREQSSRA |
| 3351 | AQTGGSITC |
| 3352 | AQGGGPRNG |
| 3353 | AQKAGAVKA |
| 3354 | AQPVSMCVS |
| 3355 | AQLSDNSIN |
| 3356 | AQQSSRGNL |
| 3357 | AQTGLCAAG |
| 3358 | AQNPVGVRG |
| 3359 | AQCDWESRV |
| 3360 | AQVMTKLGP |
| 3361 | AQYSPSVLR |
| 3362 | AQLSDARRY |
| 3363 | AQTTLRQVT |
| 3364 | AQKSSLSSV |
| 3365 | AQLRIQPAC |
| 3366 | AQKGPMRAL |
| 3367 | AQQMYEREF |
| 3368 | AQSRSLTPN |
| 3369 | AQFNRLSDS |
| 3370 | AQVGSEAGS |
| 3371 | AQTLTSSHA |
| 3372 | AQSQRVVIT |
| 3373 | AQGPPHRVS |
| 3374 | AQLHICDGF |
| 3375 | AQKFKNACV |
| 3376 | AQFVTSMKC |
| 3377 | AQGAKCSSP |
| 3378 | AQIQSPVGV |
| 3379 | AQESLMKGS |
| 3380 | AQCSTGDLG |
| 3381 | AQISRGSVP |
| 3382 | AQRIRDSET |
| 3383 | AQVTINQYG |
| 3384 | AQWPWEVDV |
| 3385 | AQRSKLGKS |
| 3386 | AQSPLRDGE |
| 3387 | AQLSSRAAP |
| 3388 | AQQCISGEA |
| 3389 | AQQDFRLGS |
| 3390 | AQRRMRPVE |
| 3391 | AQDKFICST |
| 3392 | AQLTKTFTP |
| 3393 | AQVKTCKVV |
| 3394 | AQMHMPVMR |
| 3395 | AQTQGANSV |
| 3396 | AQAISGLKC |
| 3397 | AQRSELSHR |
| 3398 | AQGSDFLGT |
| 3399 | AQCNELVVR |
| 3400 | AQNQGTLRS |
| 3401 | AQTVPHVKG |
| 3402 | AQDTLVKHC |
| 3403 | AQQYTRKYV |
| 3404 | AQNSAMRLT |
| 3405 | AQMYTTHNV |
| 3406 | AQFLTKCGS |
| 3407 | AQTHSEDRN |
| 3408 | AQAEEECDW |
| 3409 | AQRGKGSST |
| 3410 | AQNESIVFF |
| 3411 | AQCDSGKLK |
| 3412 | AQLCTGGRY |
| 3413 | AQTAVLGVP |
| 3414 | AQNCMSNGG |
| 3415 | AQTCHKPSA |
| 3416 | AQMQIHNCP |
| 3417 | AQSYDLKGH |
| 3418 | AQGGSTECM |
| 3419 | AQGSYSYVT |
| 3420 | AQSVIKPRS |
| 3421 | AQHGMLAHF |
| 3422 | AQSVHFRCG |
| 3423 | AQLLHDSVK |
| 3424 | AQLHMSPRG |
| 3425 | AQIIIKMLR |
| 3426 | AQAIETRTE |
| 3427 | AQCWEVDKR |
| 3428 | AQYERFKQV |
| 3429 | AQSDLPYSV |
| 3430 | AQIDATGRC |
| 3431 | AQLYELMAK |
| 3432 | AQVLDNFCP |
| 3433 | AQTDCKPRS |
| 3434 | AQFVPCLGT |
| 3435 | AQWWQKCKQ |
| 3436 | AQLDSHSPT |
| 3437 | AQSFTTYSL |
| 3438 | AQVIESHAA |
| 3439 | AQKNDRCSP |
| 3440 | AQERGTCLE |
| 3441 | AQAAVASVN |
| 3442 | AQQASCGAV |
| 3443 | AQVIPYCSS |
| 3444 | AQVTSNGCT |
| 3445 | AQCLSRAGK |
| 3446 | AQKSTQMYM |
| 3447 | DGDNYAERG |
| 3448 | DGEADRWLS |
| 3449 | DGVYGLEAI |
| 3450 | DGQNSLSFK |
| 3451 | DGTTYYLYS |
| 3452 | DGLKLILKG |
| 3453 | DGSSSYEGQ |
| 3454 | DGDNYTITP |
| 3455 | DGMNDASLF |
| 3456 | DGKVGQSRE |
| 3457 | DGMTGLSKP |
| 3458 | DGFVFSDAL |
| 3459 | DGSGRDTPR |
| 3460 | DGSVVSGMQ |
| 3461 | DGLSPGRFL |
| 3462 | DGCHPYHAP |
| 3463 | DGRSKRQPS |
| 3464 | DGADDISTY |
| 3465 | DGLISRHGA |
| 3466 | DGIPRSADH |
| 3467 | DGEEAPWKM |
| 3468 | DGWIENDSA |
| 3469 | DGNLCIKTY |
| 3470 | DGDEMRRDV |
| 3471 | DGTRRATPD |
| 3472 | DGKQYVKSA |
| 3473 | DGPKNYVPP |
| 3474 | DGKWVQPDT |
| 3475 | DGDSFADLY |
| 3476 | DGRVYVSLK |
| 3477 | DGNRNNNTL |
| 3478 | DGVHRNQVD |
| 3479 | DGKVPKSQL |
| 3480 | DGAKTNGYD |
| 3481 | DGLIGHTKA |
| 3482 | DGSARYTGN |
| 3483 | DGMQRIAHL |
| 3484 | DGMRKTLEN |
| 3485 | DGYDDECMP |
| 3486 | DGRLPCAMI |
| 3487 | DGLFAEMTN |
| 3488 | DGESVRPMR |
| 3489 | DGIEGTWRS |
| 3490 | DGYDDSSDY |
| 3491 | DGNHITEEI |
| 3492 | DGRRILIDA |
| 3493 | DGTKHKSKV |
| 3494 | DGRFKDVGF |
| 3495 | DGVHNSRPE |
| 3496 | DGYNEEWRD |
| 3497 | DGTNVTRKP |
| 3498 | DGKYAACEG |
| 3499 | DGYDRSYAG |
| 3500 | DGHHGNEPW |
| 3501 | DGGGSMSTW |
| 3502 | DGLMTHVDC |
| 3503 | DGCMMRLPQ |
| 3504 | DGYADVSSN |
| 3505 | DGPAIHVPR |
| 3506 | DGFRSSIGT |
| 3507 | DGQHPVRQG |
| 3508 | DGSNKRLVN |
| 3509 | DGVAGQMRA |
| 3510 | DGVSWKISE |
| 3511 | DGRTRFLEA |
| 3512 | DGDELPVVD |
| 3513 | DGDGREKFG |
| 3514 | DGKKRPYAH |
| 3515 | DGDHVSAVR |
| 3516 | DGYYRETEA |
| 3517 | DGPTRMPEI |
| 3518 | DGCLKVMRC |
| 3519 | DGGHENSCK |
| 3520 | DGGTGSLCD |
| 3521 | DGSAENQEW |
| 3522 | DGWLRCAGR |
| 3523 | DGIIEAWRD |
| 3524 | DGLEQRGGT |
| 3525 | DGGFMPRHG |
| 3526 | DGYFCRECV |
| 3527 | DGTMQSKRR |
| 3528 | DGPSQNMCT |
| 3529 | DGKSNMRIP |
| 3530 | DGHNWGRAE |
| 3531 | DGSFGFFRT |
| 3532 | DGSVTPKNQ |
| 3533 | DGKKRASAG |
| 3534 | DGDGYLYKG |
| 3535 | DGRMMSLPL |
| 3536 | DGVGGRNKK |
| 3537 | DGRVVKALP |
| 3538 | DGPWQPMDS |
| 3539 | DGCKSTKVT |
| 3540 | DGREPGDAH |
| 3541 | DGFVKGPSV |
| 3542 | DGRRSLDIL |
| 3543 | DGDIHSFSS |
| 3544 | DGSQNVTKW |
| 3545 | DGSGRSGMC |
| 3546 | DGCHKPNSS |
| 3547 | DGFLIKRSD |
| 3548 | DGGASGYWC |
| 3549 | DGFFSEPPG |
| 3550 | DGSAYWYDV |
| 3551 | DGNYSSDTC |
| 3552 | DGHVSKPDP |
| 3553 | DGTGLKHPL |
| 3554 | DGGFMAEIE |
| 3555 | DGINHQKYE |
| 3556 | DGLNKMNLN |
| 3557 | DGIARVPMH |
| 3558 | DGAQARGLN |
| 3559 | DGQLTSYRC |
| 3560 | DGKSGMFKA |
| 3561 | DGSEVRPRS |
| 3562 | DGCTRGNNI |
| 3563 | DGAQCRSMH |
| 3564 | DGLLINFCV |
| 3565 | DGVPGWGWR |
| 3566 | DGLILKRTR |
| 3567 | DGCMQSREK |
| 3568 | DGHKTRVVV |
| 3569 | DGRSKLAVH |
| 3570 | DGGQVKMWL |
| 3571 | DGRLSRVRL |
| 3572 | DGTPYRNTR |
| 3573 | DGTKKWCAD |
| 3574 | DGNSGRFST |
| 3575 | DGVQHRNST |
| 3576 | DGNRILGLT |
| 3577 | DGPACATNH |
| 3578 | DGACSSFTL |
| 3579 | DGCDALFKR |
| 3580 | DGGNSPIWK |
| 3581 | DGKSFATAR |
| 3582 | DGGLFQRTQ |
| 3583 | DGCTRSPNL |
| 3584 | DGYVTSERS |
| 3585 | DGRRSVGLR |
| 3586 | DGVTTAKWG |
| 3587 | DGVSIRKSR |
| 3588 | DGNKTRSMT |
| 3589 | DGIRPAQAQ |
| 3590 | DGQYMSKNT |
| 3591 | DGSPLDRCG |
| 3592 | DGYKYDMCV |
| 3593 | DGNCNMGTV |
| 3594 | DGKKVRMNA |
| 3595 | DGRKMRHIM |
| 3596 | DGRSPCVVM |
| 3597 | DGEFALDRS |
| 3598 | DGGRALGRF |
| 3599 | DGGKIKNKS |
| 3600 | DGRSTIRPS |
| 3601 | DGVRLVNKG |
| 3602 | DGVRLTLSG |
| 3603 | DGSKRDSTL |
| 3604 | DGAVLDGAI |
| 3605 | DGEGYDYVD |
| 3606 | DGQCKSLFQ |
| 3607 | DGVNRERTS |
| 3608 | DGSILKHHN |
| 3609 | DGMGRQTTP |
| 3610 | DGRALGTNK |
| 3611 | DGRSGGDGR |
| 3612 | DGYHKTGII |
| 3613 | DGYESRKSG |
| 3614 | DGKVALPTF |
| 3615 | DGRKVPSHV |
| 3616 | DGYARVNNG |
| 3617 | DGAWSDPNE |
| 3618 | DGVKHLHRE |
| 3619 | DGRNSRVPA |
| 3620 | DGKSMTVRS |
| 3621 | DGVPTSVWE |
| 3622 | DGMKKNLST |
| 3623 | DGAKPGFKY |
| 3624 | DGGREMKYI |
| 3625 | DGSVRASPF |
| 3626 | DGKWSHGSN |
| 3627 | DGKGRLFTQ |
| 3628 | DGKLNFANK |
| 3629 | DGLNSLMRG |
| 3630 | DGFANKVNR |
| 3631 | DGQKTRMTT |
| 3632 | DGRSLTASK |
| 3633 | DGSRFNITN |
| 3634 | DGCGYTKCD |
| 3635 | DGRMRTDLY |
| 3636 | DGSSSYGCF |
| 3637 | DGPTNIAKK |
| 3638 | DGRVQKIQA |
| 3639 | DGDRRDSIR |
| 3640 | DGHRVERFN |
| 3641 | DGMRSSSAL |
| 3642 | DGSLVSFKT |
| 3643 | DGKLMLLPK |
| 3644 | DGNLTMSRQ |
| 3645 | DGSWGARMG |
| 3646 | DGPKKMNEP |
| 3647 | DGVFNHLKS |
| 3648 | DGLQPVRAR |
| 3649 | DGLNRRQES |
| 3650 | DGTVSFGRS |
| 3651 | DGIRLVPEV |
| 3652 | DGFDRSEYR |
| 3653 | DGKFGAGKL |
| 3654 | DGSPYARQS |
| 3655 | DGGVFTTRA |
| 3656 | DGTSKAVQK |
| 3657 | DGSLYKADS |
| 3658 | DGVKNRGLA |
| 3659 | DGPIMMQCE |
| 3660 | DGFSSVMTR |
| 3661 | DGIMKAQAQ |
| 3662 | DGSNSPYSK |
| 3663 | DGMGRRQNE |
| 3664 | DGNWERSQQ |
| 3665 | DGLKGVTSS |
| 3666 | DGRNVTIMK |
| 3667 | DGEVRYRAD |
| 3668 | DGNKLQMSG |
| 3669 | DGQSPIGLV |
| 3670 | DGTHKSAWG |
| 3671 | AQSVTPKNQ |
| 3672 | AQRRMKPLD |
| 3673 | AQLVAGRDL |
| 3674 | AQRAHVGNG |
| 3675 | AQMKASSTA |
| 3676 | DGRSPNLSN |
| 3677 | AQGKYTAWE |
| 3678 | DGTEWHVGL |
| 3679 | AQQSPIQSL |
| 3680 | AQSVIGMSG |
| 3681 | AQTRVNYQK |
| 3682 | AQEHTSELQ |
| 3683 | DGRLGAVNL |
| 3684 | DGLIYEYEH |
| 3685 | DGFIYDYKH |
| 3686 | DGQSVVKLG |
| 3687 | DGGAPKCAM |
| 3688 | AQRVRNGGY |
| 3689 | DGDEYFERP |
| 3690 | AQTVLHMGE |
| 3691 | AQGRYTSLE |
| 3692 | DGDEYLVRP |
| 3693 | DGDFYLLKP |
| 3694 | AQGKYRASD |
| 3695 | AQAMVQMLG |
| 3696 | AQAVYAAMD |
| 3697 | AQTVQIQTY |
| 3698 | AQAMVGMGV |
| 3699 | DGDNYILKG |
| 3700 | AQAPIKLGE |
| 3701 | AQSIFRNGE |
| 3702 | AQSPVQMSA |
| 3703 | DGDNYMITE |
| 3704 | AQGKYSYRD |
| 3705 | DGQDPVRKT |
| 3706 | DGDNYFLKV |
| 3707 | DGDNYHVPP |
| 3708 | AQSVVALGS |
| 3709 | AQMQSPIAV |
| 3710 | DGVVGHVKG |
| 3711 | AQQNPVATV |
| 3712 | DGDQYTQEP |
| 3713 | AQGKYTHND |
| 3714 | AQSIVRLGD |
| 3715 | AQTEWHVGL |
| 3716 | AQTVVQYGE |
| 3717 | DGQQRPSLT |
| 3718 | AQGPIRDGA |
| 3719 | DGDKYFLKP |
| 3720 | DGDNYSHLP |
| 3721 | DGDNYKYVG |
| 3722 | AQGMIGMGS |
| 3723 | DGLLGMYKP |
| 3724 | AQAVVRIDG |
| 3725 | AQMMVGLGQ |
| 3726 | AQHVMNQIS |
| 3727 | DGDNYLLKT |
| 3728 | AQGRETYMD |
| 3729 | DGMYPYIGS |
| 3730 | DGDFRTIVQ |
| 3731 | DGDDYFVKE |
| 3732 | AQCVSLCSA |
| 3733 | DGDNYFIKL |
| 3734 | DGDEYLTAP |
| 3735 | AQSPVGIGT |
| 3736 | DGDYYFNKP |
| 3737 | DGDEYIIRE |
| 3738 | DGLNGMFKG |
| 3739 | DGTQSITSW |
| 3740 | DGGRYTSLE |
| 3741 | DGDNYQVRS |
| 3742 | DGFRFQDRD |
| 3743 | AQHFPRMVD |
| 3744 | DGNQHISSW |
| 3745 | AQARYTALD |
| 3746 | AQPAHGVDR |
| 3747 | AQTIYEQRS |
| 3748 | AQSTTAQYN |
| 3749 | AQGRYTSWA |
| 3750 | DGIYTAWSR |
| 3751 | AQNVVAMGY |
| 3752 | DGCVATPRH |
| 3753 | AQTMSQWGE |
| 3754 | DGDNRLHYP |
| 3755 | DGILYGRLT |
| 3756 | AQSVMLQLN |
| 3757 | AQHLLRQVD |
| 3758 | AQGFVRMDT |
| 3759 | AQCVVMDRC |
| 3760 | DGDKYFYKE |
| 3761 | AQGVIREDS |
| 3762 | DGVWLNIET |
| 3763 | AQQSPVKFS |
| 3764 | DGFIVSHNV |
| 3765 | DGYQSPFMN |
| 3766 | DGERGITKA |
| 3767 | AQSNYTALG |
| 3768 | DGDYRTVKQ |
| 3769 | AQGSGLQMF |
| 3770 | AQGMVGLGF |
| 3771 | DGDYRVTKQ |
| 3772 | AQTEYWNER |
| 3773 | AQSPVKLGY |
| 3774 | DGDYYVQKG |
| 3775 | DGKVGQTKT |
| 3776 | DGIRGQEKA |
| 3777 | AQQPTKLGS |
| 3778 | DGDMYLEVA |
| 3779 | AQDMLRLGM |
| 3780 | AQGVVAYLT |
| 3781 | AQNVYSPVL |
| 3782 | AQEKPGQVK |
| 3783 | AQTIVWQID |
| 3784 | AQINGMSKG |
| 3785 | DGMNYFGDL |
| 3786 | AQCDWSHMD |
| 3787 | AQTTGYQYS |
| 3788 | AQTPVKLSH |
| 3789 | AQGKYMPRD |
| 3790 | AQGMSLLSK |
| 3791 | AQCMVAIGC |
| 3792 | DGRAGQNKG |
| 3793 | DGLRGNTYL |
| 3794 | DGSQDTTTW |
| 3795 | DGRTGMLKP |
| 3796 | AQTPGLNKG |
| 3797 | DGEGFYCAK |
| 3798 | AQTPVAAGC |
| 3799 | DGKSGQNKS |
| 3800 | DGGETHVWK |
| 3801 | AQCDSIYCL |
| 3802 | AQICCKSPC |
| 3803 | DGSGPIRMG |
| 3804 | AQKVTGFGS |
| 3805 | AQLRKANRP |
| 3806 | DGKPGMLKV |
| 3807 | AQVRHHSDN |
| 3808 | AQTPIRMGP |
| 3809 | AQSPIRVGT |
| 3810 | AQQDPVRKT |
| 3811 | AQTVLNQMK |
| 3812 | AQSGPIRMG |
| 3813 | DGDFYKCEP |
| 3814 | DGLKLISKG |
| 3815 | AQTVTFQTC |
| 3816 | AQDSEYFCD |
| 3817 | AQRARDRHI |
| 3818 | AQQCKVSVI |
| 3819 | AQSWTYRRF |
| 3820 | AQGPVRANG |
| 3821 | AQGKRCAIE |
| 3822 | AQSLQLFGM |
| 3823 | AQVGDVRLK |
| 3824 | AQTMTKFGT |
| 3825 | AQPCIRDRG |
| 3826 | DGKTGHYKV |
| 3827 | DGFSTFINK |
| 3828 | AQIL YGRLT |
| 3829 | AQEYSLSFK |
| 3830 | AQERIGQFK |
| 3831 | AQRVKKVPG |
| 3832 | DGQMVTRFG |
| 3833 | AQLLAWFIL |
| 3834 | AQSTYWDRC |
| 3835 | AQRNAKLKM |
| 3836 | AQSPYKLGS |
| 3837 | AQGKDCLLP |
| 3838 | AQSATCQRS |
| 3839 | AQCASICNF |
| 3840 | AQAIRGARA |
| 3841 | AQTVYWMPA |
| 3842 | AQHRNCQVD |
| 3843 | AQQNPVKCS |
| 3844 | DGLKLVFKP |
| 3845 | DGGNCMHSC |
| 3846 | AQTVIKYGA |
| 3847 | DGIKLIVKS |
| 3848 | AQGCRALTS |
| 3849 | AQNRCSNVN |
| 3850 | AQHVPGKKQ |
| 3851 | AQCVAAMKC |
| 3852 | AQSFIKLGQ |
| 3853 | AQAGSYDCV |
| 3854 | AQAMAARSS |
| 3855 | AQTRWHGNE |
| 3856 | AQVKRQLRC |
| 3857 | AQKPIGFGA |
| 3858 | AQDCRIVKQ |
| 3859 | AQCVVVTRC |
| 3860 | AQUIYVRND |
| 3861 | DGFMLGRCP |
| 3862 | DGLARLIHL |
| 3863 | AQPFFSECL |
| 3864 | DGKAGQIKY |
| 3865 | AQSSFSKNF |
| 3866 | AQGPVKMGF |
| 3867 | AQSVVRMSA |
| 3868 | AQGCQWMPK |
| 3869 | AQRILKGMG |
| 3870 | AQSPENCGA |
| 3871 | AQLWRQQVA |
| 3872 | AQSVVKLGN |
| 3873 | AQDKPGQVK |
| 3874 | AQVPQLIKG |
| 3875 | AQTISTGIY |
| 3876 | DGFGFTLST |
| 3877 | AQWQSPVHS |
| 3878 | AQNPVARSD |
| 3879 | AQKWDNQQS |
| 3880 | DGIFGGGGK |
| 3881 | AQTMLRMGE |
| 3882 | AQSMLKLGE |
| 3883 | AQISGRMAA |
| 3884 | AQTVLFQVE |
| 3885 | AQTKSNGIS |
| 3886 | AQGRYTASE |
| 3887 | DGIKGGGSK |
| 3888 | AQSVLSQVS |
| 3889 | AQADPLHKG |
| 3890 | AQTTLSQVT |
| 3891 | AQTVLRSGE |
| 3892 | AQSPFKEGL |
| 3893 | AQSPVGLGE |
| 3894 | AQTTVHQSS |
| 3895 | DGCQPPKCI |
| 3896 | AQTVLHQTH |
| 3897 | AQERYTSHN |
| 3898 | AQHTTHQVS |
| 3899 | AQTVVGMGI |
| 3900 | AQSVVGLGA |
| 3901 | AQTVNYQVT |
| 3902 | AQQVIKGGE |
| 3903 | AQTWDTGIS |
| 3904 | AQPMLDRNT |
| 3905 | DGMGPIRMS |
| 3906 | AQTVLFQQS |
| 3907 | AQTVNFQIT |
| 3908 | AQTTLQQIT |
| 3909 | DGIAEEDWM |
| 3910 | AQNQSPVGF |
| 3911 | AQSTVMQIS |
| 3912 | AQSITGLGY |
| 3913 | AQVIGHNLV |
| 3914 | DGDNRVTRE |
| 3915 | DGQSPIMRM |
| 3916 | DGTQSPTSW |
| 3917 | DGVWLNLSS |
| 3918 | AQAKPMFKC |
| 3919 | AQGFQLPHL |
| 3920 | AQIVTNQYG |
| 3921 | AQGVIRMGD |
| 3922 | DGAWTGAPK |
| 3923 | AQHTLSQMS |
| 3924 | AQGKYAAIC |
| 3925 | AQENDMVFF |
| 3926 | DGWYEPGSS |
| 3927 | AQLSAFTSA |
| 3928 | DGKDGHNKG |
| 3929 | DGKYEPPAC |
| 3930 | DGRSENDMD |
| 3931 | DGEDRFGRD |
| 3932 | DGLTYYGIE |
| 3933 | DGGNYVVYK |
| 3934 | DGIVRPTYG |
| 3935 | AQFIASHNV |
| 3936 | AQPSSISYL |
| 3937 | AQGSGLNIM |
| 3938 | DGDTYALGY |
| 3939 | DGKDGHWKP |
| 3940 | AQTMVKGGP |
| 3941 | AQVVYDREF |
| 3942 | AQTRFIEPY |
| 3943 | AQPLVTHNI |
| 3944 | AQSTTYQFS |
| 3945 | AQKPTKLGE |
| 3946 | AQHGSYALA |
| 3947 | AQNPTKMGM |
| 3948 | AQMVYDRDF |
| 3949 | AQGNSLSYQ |
| 3950 | AQNPVRNGA |
| 3951 | AQSMVKGGS |
| 3952 | AQSMVKGGP |
| 3953 | AQVYGLMEN |
| 3954 | AQHTKFAYD |
| 3955 | AQSVHYQVS |
| 3956 | AQTVVKMGV |
| 3957 | AQDGSLTFR |
| 3958 | AQSIHSHNT |
| 3959 | AQILRIEPT |
| 3960 | DGGMSPVWK |
| 3961 | AQSYTHPDN |
| 3962 | AQTVRNQVS |
| 3963 | AQSVVRDKG |
| 3964 | AQNVLQMGK |
| 3965 | DGILGLFKG |
| 3966 | DGRLECCNF |
| 3967 | AQQFASHNV |
| 3968 | AQMDGLWKG |
| 3969 | DGRIGHSKP |
| 3970 | AQGRHSACE |
| 3971 | AQYRNGGAS |
| 3972 | AQTVVKLGS |
| 3973 | AQSTTWQTS |
| 3974 | AQSVVKLGT |
| 3975 | AQQNSLSYK |
| 3976 | DGMRSGALN |
| 3977 | AQTRMSGIS |
| 3978 | AQGSSLPFK |
| 3979 | DGDLKWNEP |
| 3980 | AQSMVGMGC |
| 3981 | DGAITNVWK |
| 3982 | AQKWDLTDR |
| 3983 | AQGMIKGGS |
| 3984 | AQGNSLKYT |
| 3985 | DGGHFVVFK |
| 3986 | DGKVGLIKH |
| 3987 | AQNVIKLGM |
| 3988 | AQALLYAHQ |
| 3989 | AQHNSLSFK |
| 3990 | AQIMSCTMF |
| 3991 | AQDSVYVNR |
| 3992 | AQTLQYQKS |
| 3993 | AQLNRLPHL |
| 3994 | AQTTLSQRT |
| 3995 | DGAWTGSRI |
| 3996 | AQTWTTGVS |
| 3997 | AQNLSLAYK |
| 3998 | AQQMCESTK |
| 3999 | AQQNSLSFK |
| 4000 | AQKWDVMDR |
| 4001 | AQYPPVFKS |
| 4002 | DGKSPHYKP |
| 4003 | AQSVVRLGV |
| 4004 | AQLNDIRRH |
| 4005 | AQGVVRMAQ |
| 4006 | AQFSGLAYK |
| 4007 | AQRWEIKPE |
| 4008 | AQIIGGRAV |
| 4009 | AQYLHGREF |
| 4010 | AQGKYQTSG |
| 4011 | AQKISSHNI |
| 4012 | DGTASSKWG |
| 4013 | AQVGEVRLK |
| 4014 | AQKLSSHNT |
| 4015 | AQLSSLSFK |
| 4016 | DGGPVAIFW |
| 4017 | AQTRWTGIS |
| 4018 | AQWSGSHNY |
| 4019 | AQGNSMSYK |
| 4020 | AQGSSMMYK |
| 4021 | DGRTCSFTN |
| 4022 | DGKGVMLYK |
| 4023 | AQLHMGRLS |
| 4024 | AQKHNTGIS |
| 4025 | AQCINGRSL |
| 4026 | AQYPAISFK |
| 4027 | AQISPGRYL |
| 4028 | AQTSSMRHL |
| 4029 | AQRVVSHNI |
| 4030 | AQGRGLSIL |
| 4031 | AQTMSRGGC |
| 4032 | AQPNPVRYC |
| 4033 | AQLTNRSSP |
| 4034 | AQALSHTNN |
| 4035 | AQIKSRQAH |
| 4036 | AQNNNPCAS |

Screening of CA-IV Binding Peptides and/or AAVs:

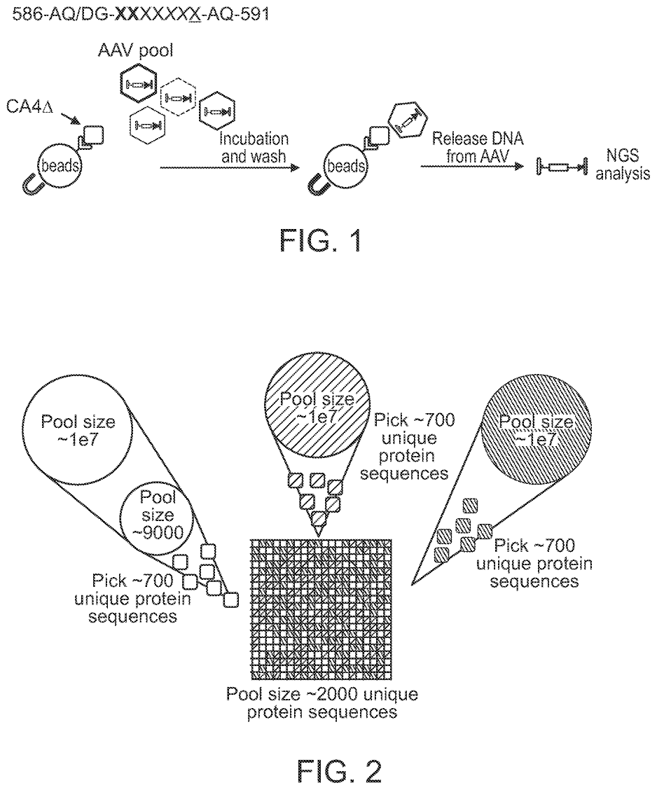

In certain aspects, the invention provides methods for screening peptides that bind to human CA4 and/or primate CA4. In certain embodiments, the methods provided in the invention utilize an animal-free work-flow for evaluating the BBB permeability of the peptides and AAVs in humans and/or primates. In certain embodiments, the evaluation is conducted by resin-based screening. An overview of the process used in the invention is provided in FIG. 1. The AAV9-based library was produced with randomized 7-amino acid peptide insertions between the 588/589 site, and either AQ (wild type) or DG at positions 587 to 588 (NNK library pool) following M-CREATE method. Ravindra Kumar, S., Miles, T. F., Chen, X., Brown, D., Dobreva, T., Huang, Q., Ding, X., Luo, Y., Einarsson, P. H., Greenbaum, A., et al. (2020). Multiplexed Cre-dependent selection yields systemic AAVs for targeting distinct brain cell types. Nat Methods 17, 541-550. 10.1038/s41592-020-0799-7.

In certain embodiments, the invention provides HA-tag modified huCA4 as a capture agent for resin-based screening for AAV/antibody engineering. In certain embodiments, the invention provides HA-tag modified rhCA4 as a capture agent for resin-based screening for AAV/antibody engineering. In certain embodiments, the rhCA4 sequences are provided in Table 2.

In certain embodiments, engineered receptor proteins are replaced the C-terminal GPI signal peptide with an HA tag, allowing attachment to anti-HA magnetic resin. The AAV library pool, anti-HA resin, with or without HA-tagged receptor (negative control), are then incubated overnight to enrich receptor-binding variants, followed by stringent washing to remove non-specific binding.

FIG. 2 provides the composition of unique CA4-binding AAVs of the invention. Three NNK library pools with a diverse distribution of AAV variants were produced independently. Two selection rounds were conducted on one NNK pool, yielding ~9,000 unique sequences in Round 1 and ~700 top CA4-binding AAVs in Round 2. Approximately 700 variants were also selected from the other two NNK pools. The final outcome consisted of two pools, containing 2005 unique AAVs with binding affinity for huCA4 and 1979 AAVs for rhCA4.

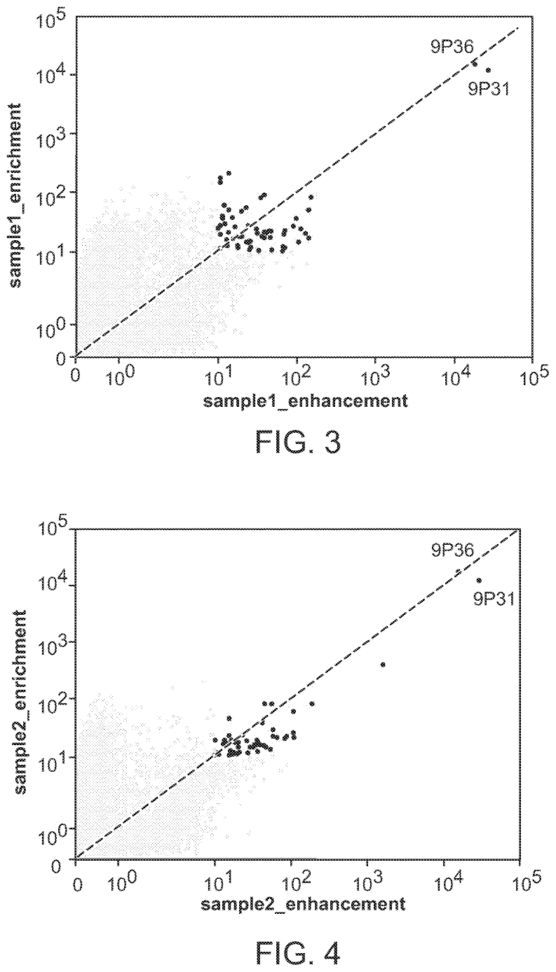

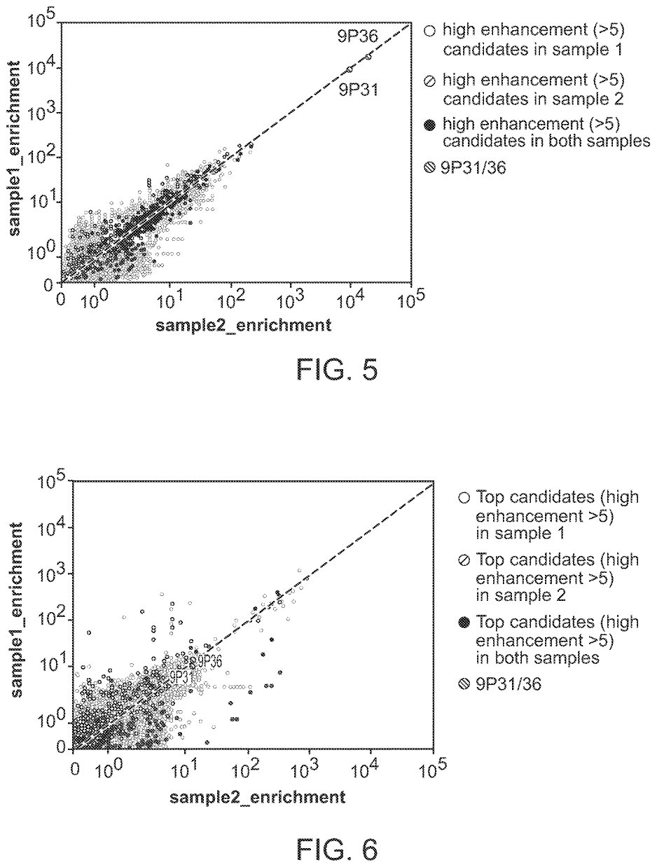

The effectiveness of this in vitro resin-based screening method was assessed for identifying promising AAV variants. A small pool of around 18,000 unique AAV variants including positive controls 9P31, 9P36 with mouse Car4 (msCar4) protein on the resin was tested. The msCar4 pull-down assay resulted in both high enrichment and enhancement of 9P31 and 9P36 (FIGS. 3 and FIG. 4), with consistent and robust performance in both low-dose (sample 1, 1.6e11 vg) and high-dose input (sample 2, 8e11 vg) (FIG. 5). In certain embodiments, the formula for calculating enrichment and enhancement are defined below.

$$Enhancement = \frac{Ri ( receptor enriched pool )}{Ri ( negative control pool )}$$

$$Enrichment = \frac{Ri ( receptor enriched pool )}{Ri ( input pool )}$$

$$R : the relative percentage of AAV variant i in a sample .$$

FIG. 3 provides msCar4 resin-based selection with a low dose of library input (1.6e11 vg). Red dots (on the top right corner of the graph) indicate 9P31 and 9P36, while orange dots represent AAV variants with enrichment>10 and enhancement>10. Yellow dots display all other AAV variants recovered on the resin.

FIG. 4 provides msCar4 resin-based selection with a high dose of library input (8e11 vg). Red dots (on the top right corner of the graph) indicate 9P31 and 9P36, while orange dots represent AAV variants with enrichment>10 and enhancement>10. Yellow dots display all other AAV variants recovered on the resin.

Notably, in certain beneficial aspects of the invention, resin-based selection offered better signal-to-noise ratios compared to cell-based selection methods by recovering AAV variants that successfully transduced HEK293 adherent cells overexpressing msCar4 (FIG. 5 and FIG. 6).

FIG. 5 provides AAV variants performance in resin-based selection at low and high dose.

FIG. 6 provides performance of AAV variants in cell-based selection at varying doses. HEK293 cells were transfected with either pUC18 (negative control) or CA4 plasmid in a 6-well plate. After two days, AAV pools of 1.25e10 vg (sample 1) or 5e10 vg (sample 2) were added and incubated for 24 hours. Cells were subsequently harvested, lysed, and RNA was extracted. RNA samples underwent RT-PCR and PCR amplification for NGS analysis.

As evident from data provided in FIG. 5 and FIG. 6, resin-based selection offered better signal-to-noise ratios compared to cell-based selection methods by recovering AAV variants that successfully transduced HEK293 adherent cells.

The selection method was validated using LY6A on the resin and the same library input pool, which includes positive controls PHP.eB and PHP.B. Huang, Q., Chan, K. Y., Tobey, I. G., Chan, Y. A., Poterba, T., Boutros, C. L., Balazs, A. B., Daneman, R., Bloom, J. M., Seed, C., and Deverman, B. E. (2019). Delivering genes across the blood-brain barrier: LY6A, a novel cellular receptor for AAV-PHP.B capsids. PLoS One 14, e0225206. 10.1371/journal.pone.0225206; Chan, K. Y., Jang, M. J., Yoo, B. B., Greenbaum, A., Ravi, N., Wu, W. L., Sanchez-Guardado, L., Lois, C., Mazmanian, S. K., Deverman, B. E., and Gradinaru, V. (2017). Engineered AAVs for efficient noninvasive gene delivery to the central and peripheral nervous systems. Nat Neurosci 20, 1172-1179. 10.1038/nn.4593.

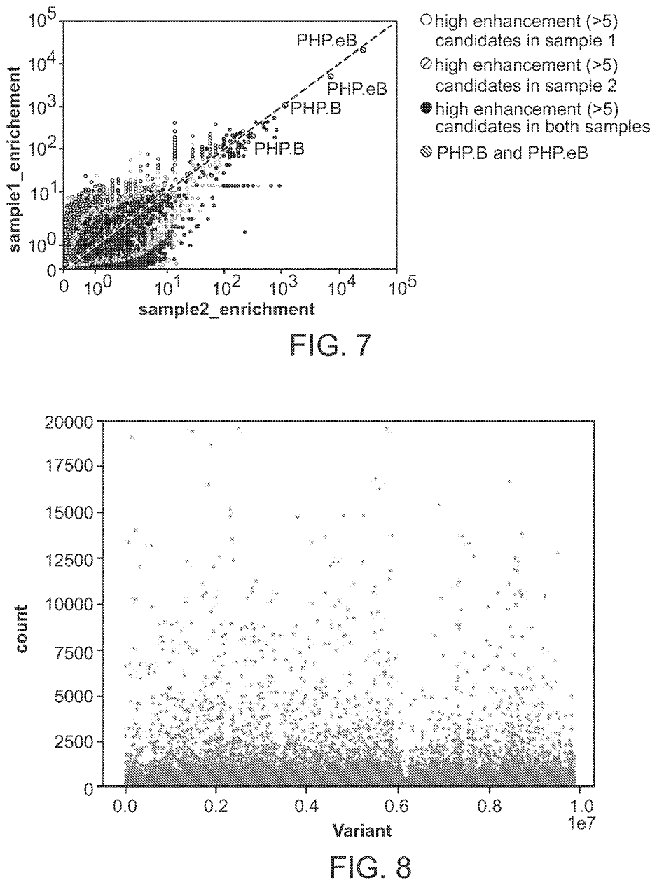

FIG. 7 provides AAV variants performance in LY6A resin-based selection across low (sample 1, 1.6e11 vg) and high dose (sample 2, 8e11 vg). Red dots (on the top right) represent PHP.eB and PHP.B (including codon replicates). Other dot color codes are provided on the right. Significantly, the selection process effectively enriched PHP.eB and PHP.B (FIG. 7).

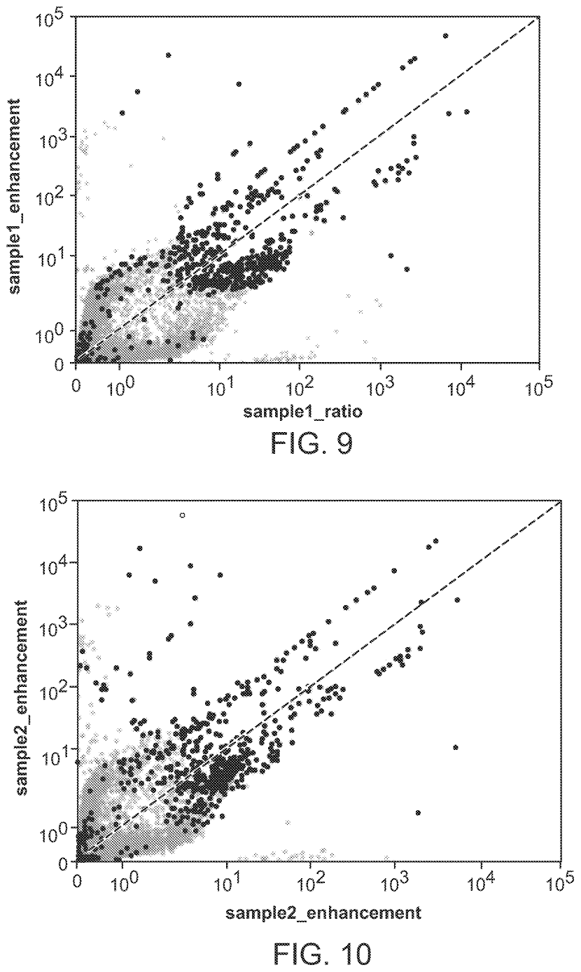

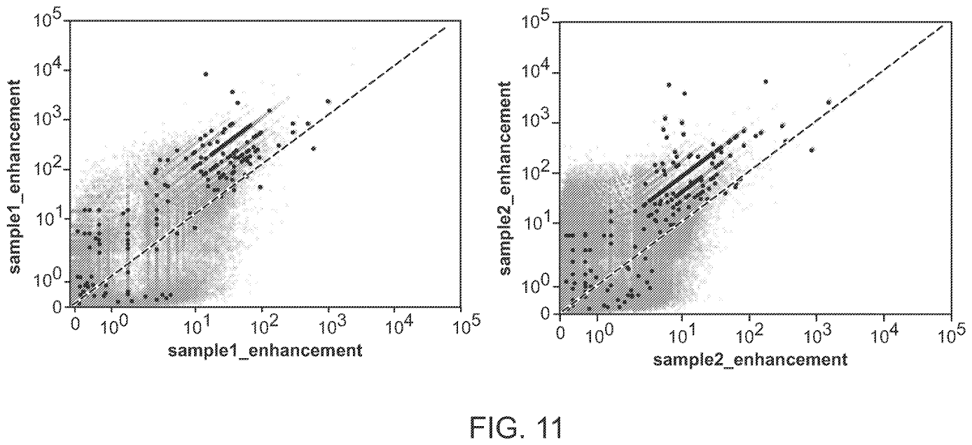

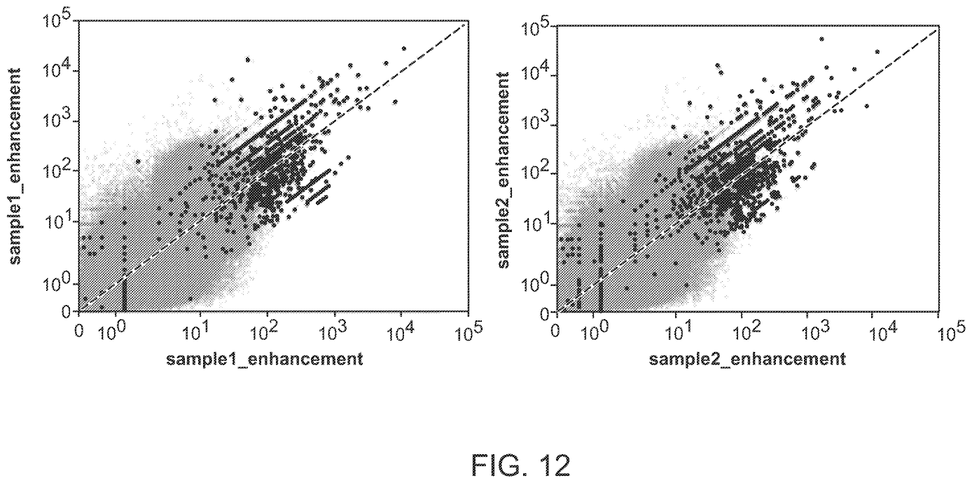

Randomized AAV library pools (AAV9 as the parental capsid) with 7-amino acid peptide insertion at 588/589 site and AQ (wild type) or DG at position 587 to 588 (NNK library pool) (FIG. 1) were generated via the evolution method provided in Kumar 2020. Considering that only ~1e7 AAV variants can be produced from 2.56e9 variants (theoretical library size, including unstable AAVs), three independent NNK library pools were generated with evenly distributed AAV variants. FIG. 8 provides the library pools generated using these methods. For one of the NNK pools, two rounds of selection was performed. Round 1 produced ~9,000 unique sequences as input for round 2, and around 700 top variants as CA4-binding AAVs (FIG. 9 and FIG. 10) were selected. FIG. 9 and FIG. 10 represent the huCA4-binding AAVs after the Round 2 selection at a low library input dose (5e10 vg) and high library input dose (2.5e11 vg) respectively. The huCA4-binding AAVs in the final pool are represented by green dots in FIGS. 9 and 10. Additionally, around 700 variants from the other two NNK pool selections were selected (FIG. 11 and FIG. 12). FIG. 11 provides data for independent round 1 selection replicate 2 for huCA4-binding AAVs at a low (sample 1, 1.6e11 vg) and high (sample 2, 8e11) library input dose. FIG. 12 provides data for independent round 1 selection replicate 3 for huCA4-binding AAVs at a low (sample 1, 1.6e11 vg) and high (sample 2, 8e11) library input dose. The huCA4-binding AAVs in the final pool are represented by green dots in FIGS. 11 and 12.

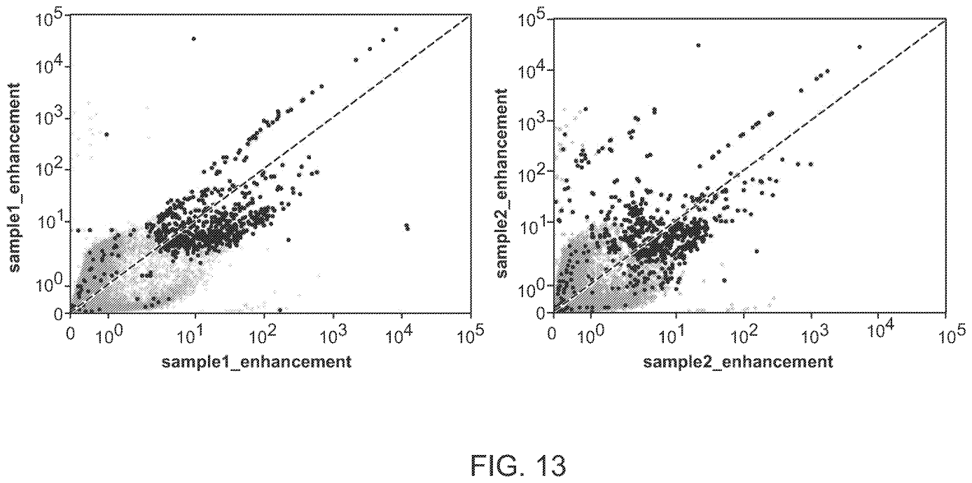

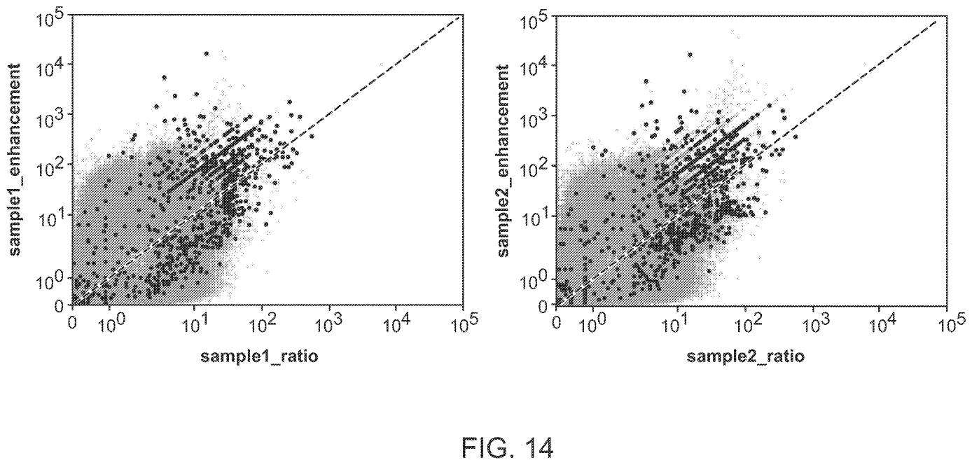

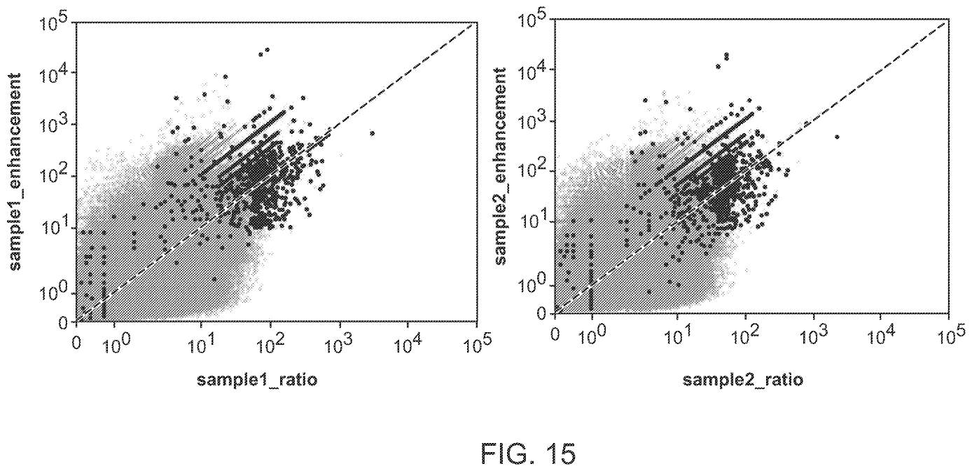

Similarly, a pull down of the pertinent rhCA4-binding AAVs was also conducted. FIG. 13 provides round 2 pulldown selection results for rhCA4-binding AAVs at a low (sample 1, 5e10 vg) and high (sample 2, 2.5e11 vg) library input dose. FIG. 14 provides independent round 1 selection replicate 2 for rhCA4-binding AAVs at a low (sample 1, 1.6e11 vg) and high (sample 2, 8e11) library input dose. FIG. 15 provides independent round 1 selection replicate 2 for rhCA4-binding AAVs at a low (sample 1, 1.6e11 vg) and high (sample 2, 8e11) library input dose. The rhCA4-binding AAVs in the final pool are represented by green dots in FIGS. 13, 14, and 15.

Ultimately, a pool of 2005 unique huCA4-binding AAVs and another pool of 1979 rhCA4-binding AAVs was obtained (FIG. 2, 13-15). The shortlisted 9-amino acid sequences after position 586 are listed in Tables 4 and 5.

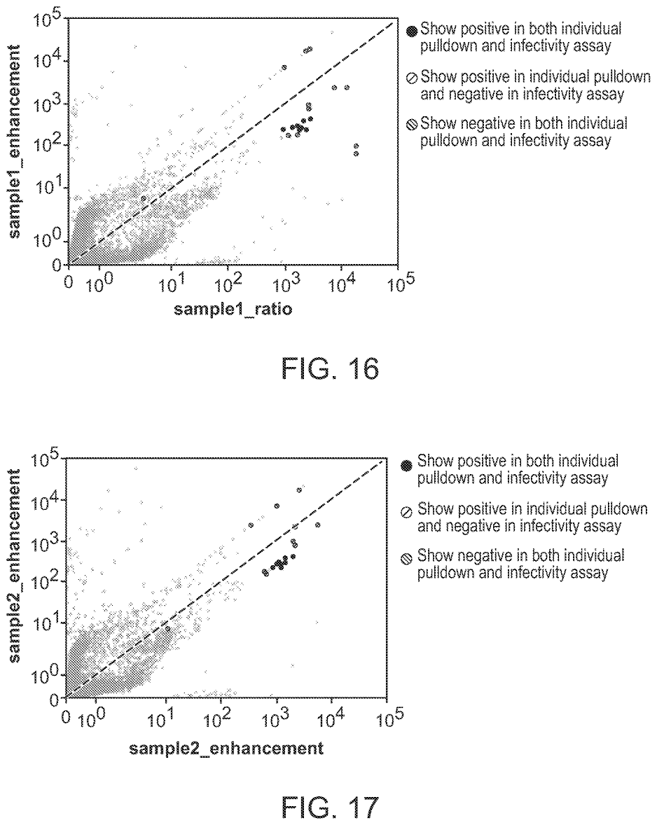

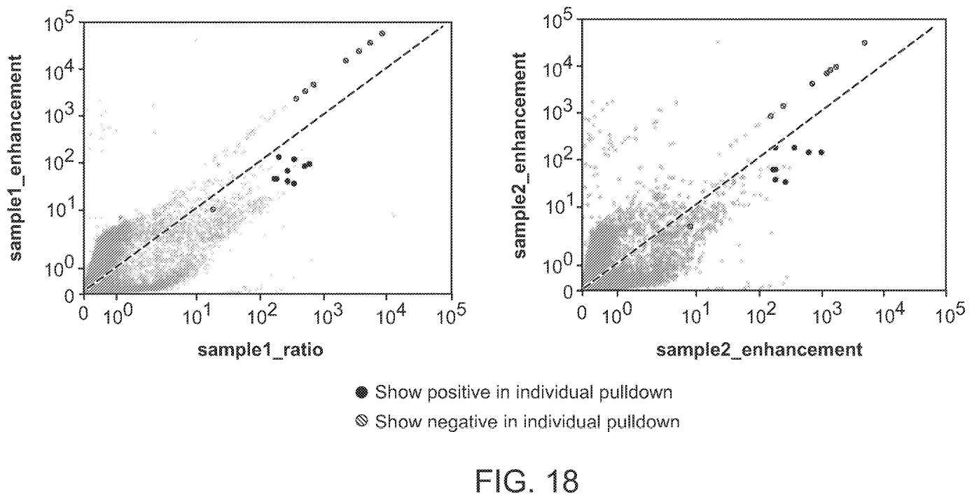

In the selected AAV variants, a panel of AAVs listed in Table 1 was selected and further individual pull-down and cell infectivity assays were performed. These experiments confirmed that AAVs bind to huCA4 or rhCA4. For the pull-down assay, each single AAV variant was incubated with anti-HA resin overnight with or without HA-tagged CA4 proteins (negative control), followed by washing the resin multiple times and eluting enriched AAV with SDS-loading buffer. The AAV quantity was assessed in the sample and negative control using western blot with anti-AAV and anti-HA antibodies. The results were validated by the pull-down assay, Alpha 33, 41, 42, 43, 44, 45, 48, 49, 50, and 52 demonstrated clear binding to huCA4. FIG. 16 provides data from round 2 pulldown selection outcomes for huCA4-binding AAVs at a low library input dose (5e10 vg). A panel of AAVs (indicated by green, purple, and blue dots; color scheme provided on the right) was chosen for individual characterization. Green dots correspond to Alpha 43, Alpha 45, Alpha 48, and Alpha 49. FIG. 17 provides data from round 2 pulldown selection outcomes for huCA4-binding AAVs at a high library input dose (2.5e11 vg). A panel of AAVs (indicated by green, purple, and blue dots; color scheme provided on the right) was chosen for individual characterization. Green dots correspond to Alpha 43, Alpha 45, Alpha 48, and Alpha 49. FIG. 18 provides data for Round 2 pulldown selection outcomes for rhCA4-binding AAVs at a low (sample 1, 5e10 vg) and high (sample 2, 2.5e11 vg) library input dose. A panel of AAVs (indicated by green and blue dots; color scheme provided on the right) was chosen for individual characterization. Green dots correspond to Omega 2, 5, 6, 8 and 11.

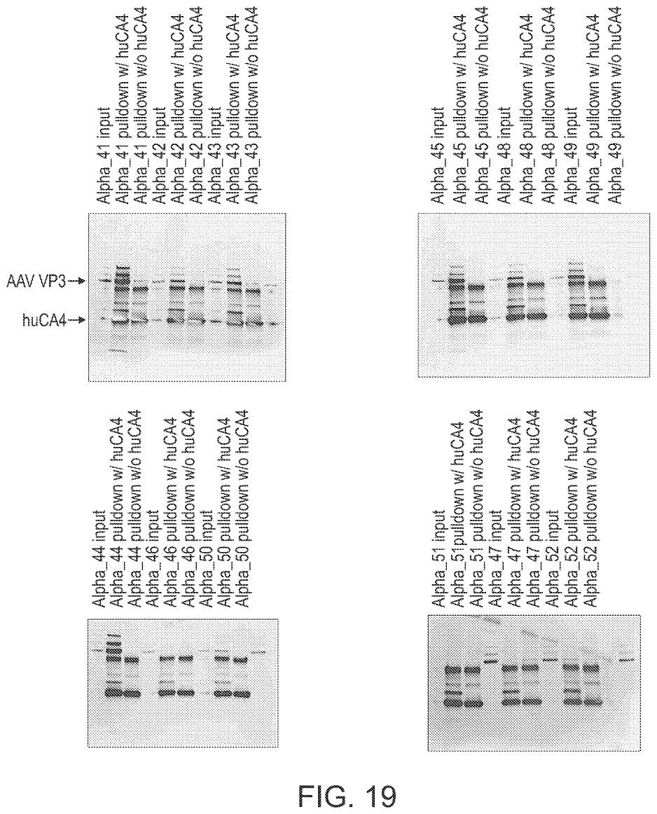

FIG. 19 provides data from pull-down assay evaluating individual AAV variants' binding with huCA4. The AAVs were incubated overnight with anti-HA resin, with or without HA-tagged huCA4 proteins (negative control). Following multiple washes, enriched AAVs were eluted using SDS-loading buffer. AAV quantity in the sample and negative control was assessed via Western blot using anti-AAV and anti-HA antibodies.

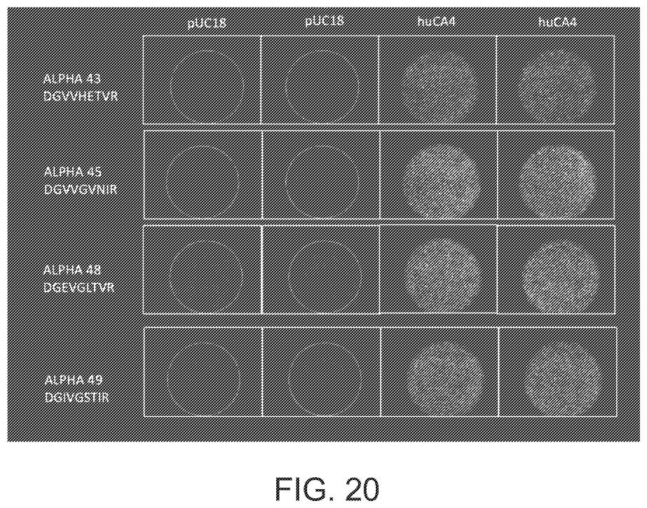

Alpha 43, 45, 48, and 49 exhibited huCA4-boosted cell transduction in cell infectivity assays. For these assays, HEK293 adherent cells were transfected with pUC18 or CA4 plasmid and subsequently transduced the cells with each AAV carrying the EGFP gene two days later After 24-hour incubation of AAV and cells, the fluorescent signal inside cells was monitored using a fluorescence microscope in a 96-well plate. FIG. 20 provides the data for the assessment of huCA4-enhanced cell transduction of Alpha 43, 45, 48, and 49.

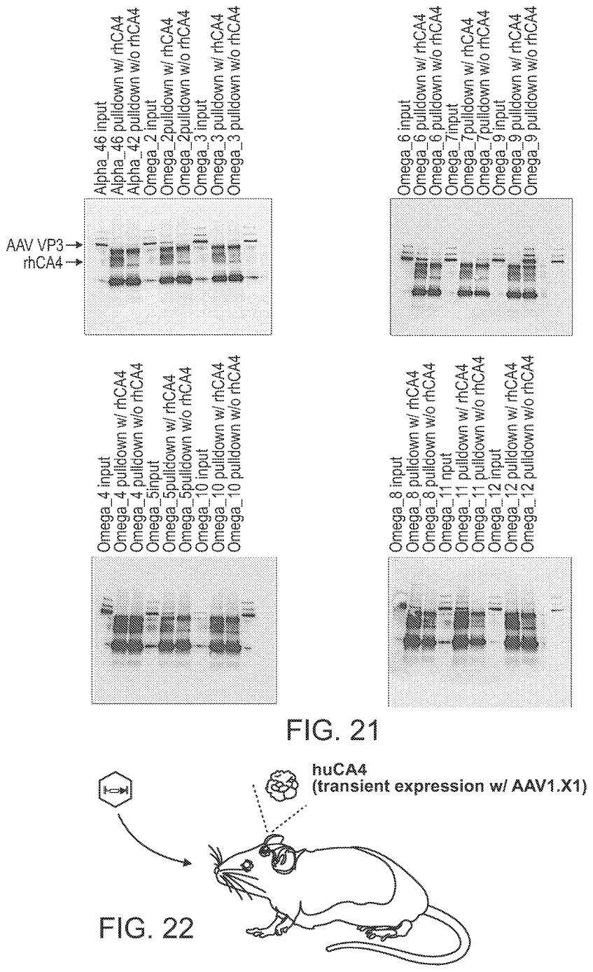

Similar experiment was conducted for verifying that Omega 2, 5, 6, 8, and 11 bound to rhCA4 in the pull-down assay. FIG. 21 provides pull-down assay evaluating individual AAV variants' binding with rhCA4. AAVs were incubated overnight with anti-HA resin, with or without HA-tagged rhCA4 proteins (negative control). Following multiple washes, enriched AAVs were eluted using SDS-loading buffer. AAV quantity in the sample and negative control was assessed via Western blot using anti-AAV and anti-HA antibodies.

Subsequently, the huCA4-binding AAVs were evaluated in mice with humanized BBB. FIG. 22 provides an overview of the mice with humanized CA4. The huCA4 gene is transiently introduced to endothelial cells using AAV1.X1, a vector specific to these cells. Three weeks later, a potential huCA4-binding AAV carrying an EGFP cargo is systemically injected to assess BBB transcytosis efficiency.

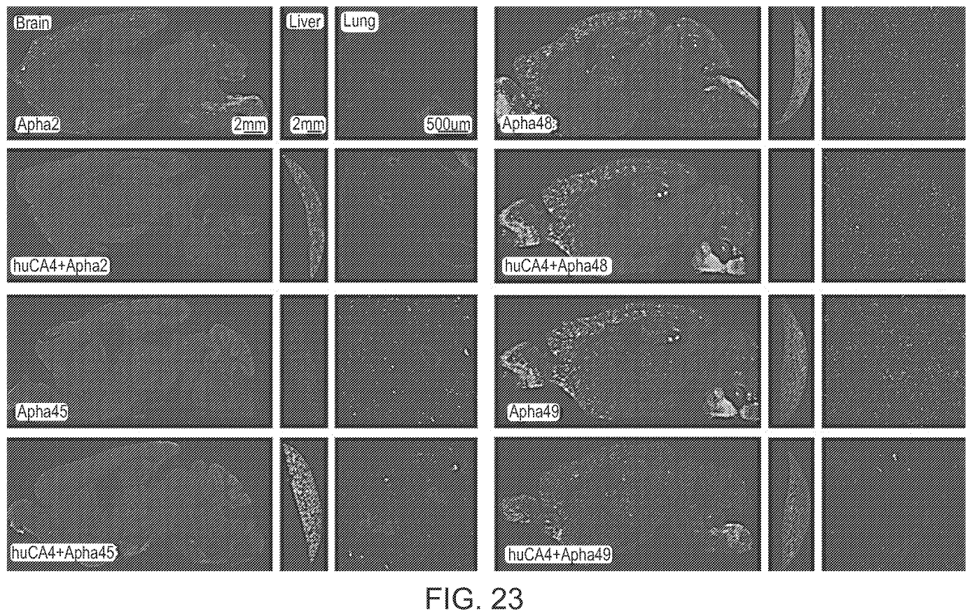

FIG. 23 provides data for huCA4-binding AAVs performance in mice. The mice were systemically injected with huCA4-binding AAV packaging EGFP only or with both AAV1.X1:CAG-huCA4 and huCA4-binding AAV. Apha2, Apha45, Apha48, Apha49 were tested. The expression shown in FIG. 23 was at 3 weeks after injection.

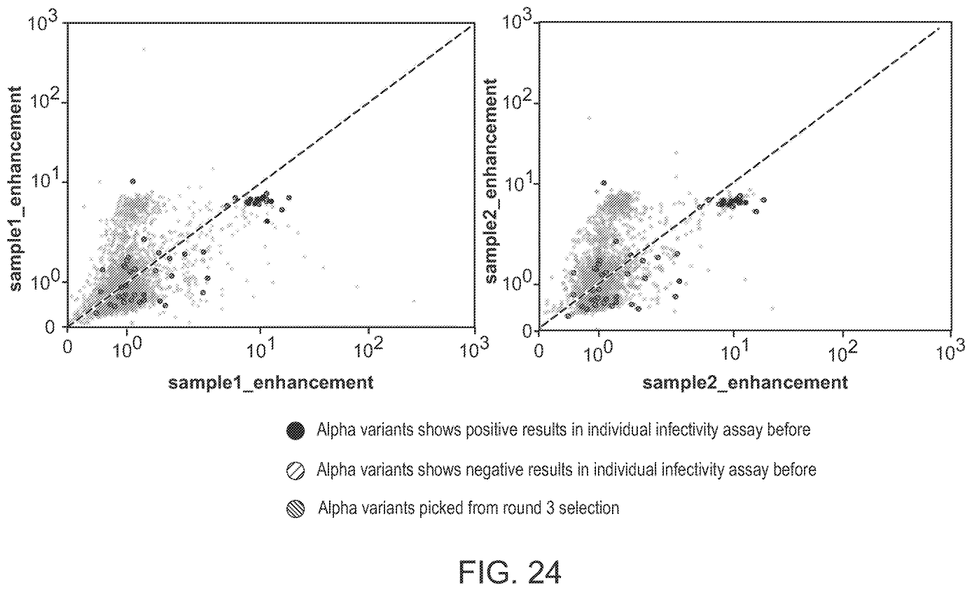

For the AAV variants pool mentioned above, we continued with a round 3 cell-based selection and pulldown selection. Alpha 41, 59-64 show promising huCA4-enhanced infectivity and huCA4-binding like the validated Alpha 43, 45, 48 and 49. FIG. 24 provides data for cell-based selection of huCA4-binding AAVs. HEK293 cells transfected with either pUC18 or huCA4 plasmid for 48 hours, then transduced with an AAV library pool of 2005 huCA4-binding variants (FIG. 2). Green dots: Alpha 43, 45, 48, and 49 with huCA4-enhanced infectivity: Blue dots: other tested variants with huCA4-independent infectivity; Red dots: Alpha 41, Alpha 59-64, selected from round 3 as individual test candidates.

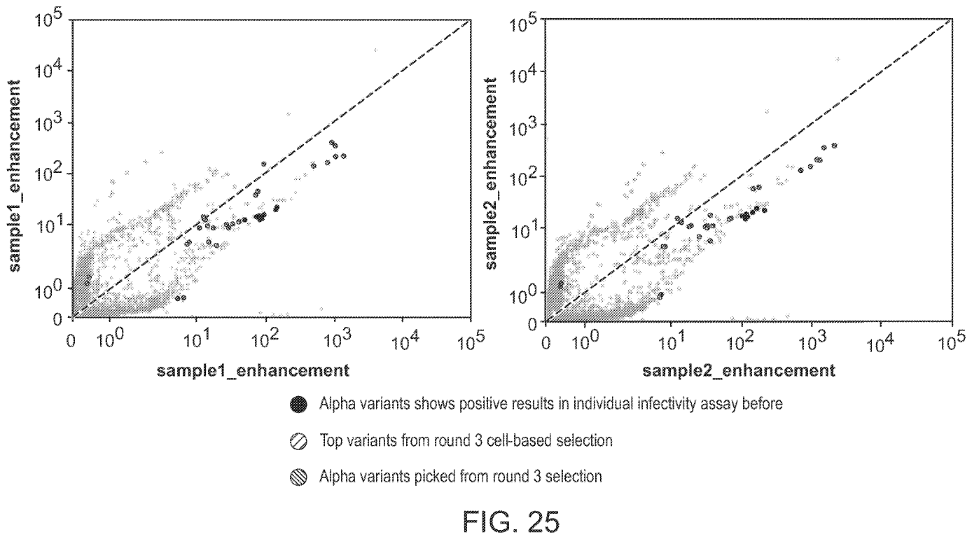

FIG. 25 provides data round 3 pulldown selection outcomes for huCA4-binding AAVs at a low (sample 1, 1.6e10 vg) and high (sample 2, 7.8e10 vg) library input dose. Green dots: Alpha 43, 45, 48, and 49 with huCA4-binding; Orange dots and red dots show top variants from round 3 cell-based selection. Among them, red dots represent Alpha 41, Alpha 59-64 which are selected for individual test.

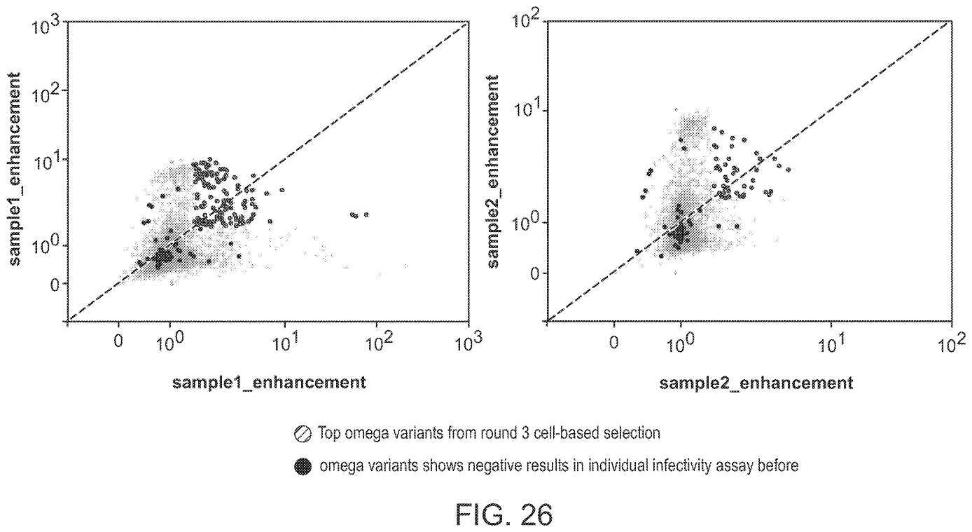

FIG. 26 provides data for cell-based selection of rhCA4-binding AAVs in round 3. HEK293 cells transfected with either pUC18 or rhCA4 plasmid for 48 hours, then transduced with an AAV library pool of 1979 rhCA4-binding variants (FIG. 2). Orange dots: top variants with rhCA4-enhanced infectivity; Blue dots: other omega variants with rhCA4-independent infectivity tested before.

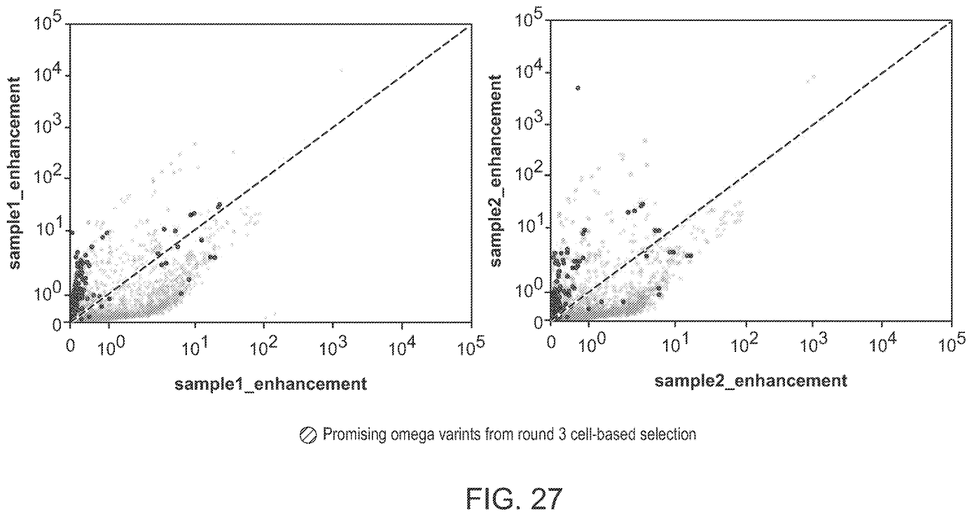

FIG. 27 provides round 3 pulldown selection outcomes for rhCA4-binding AAVs at a low (sample 1, 1.6e10 vg) and high (sample 2, 7.8e10 vg) library input dose. Orange dots: top variants from round 3 cell-based selection. Among them, Omega 33-52 are chosen for individual testing.

### Delivery of Therapeutic Cargo Across BBB

The invention beneficially recognizes that systemic delivery of adeno-associated virus (AAVs), as opposed to direct brain or eye injection, offers a non-invasive approach with broader distribution, which is particularly beneficial for diffuse neurological disorders.

In some embodiments, the payload or therapeutic cargo to be delivered to a nervous system is a biological molecule, a non-biological molecule, or a combination thereof. In some embodiments, the biological molecule is selected from the group consisting of a nucleic acid sequence, a protein, a peptide, a lipid, a polysaccharide, and any combination thereof. In some embodiments, the payload is a therapeutic molecule. In some embodiments, the nucleic acid sequence to be delivered to a nervous system comprises one or more of: a) a sequence encoding a trophic factor, a growth factor, or other soluble factors that might be released from the transduced cells and affect the survival or function of that cell and/or surrounding cells; b) a DNA that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

Accordingly, in certain aspects, the invention provides methods of increasing permeability of the blood brain barrier for delivery of therapeutic cargo across the BBB to the central nervous system and/or eye. In certain embodiments, the invention provides AAVs binding to rhesus CA4 (rhCA4) or human CA4 (huCA4) for CNS therapeutic cargo delivery in these species for both preclinical and clinical applications. In other beneficial aspect of the invention, CA4 is also highly concentrated in the primate brain, eye, and gut compared to other reported transporters such as transferrin receptor with broader expression patterns. Thus, the methods provided in the invention for therapeutic cargo delivery across in the CNS may have reduced off-target organ delivery.

In certain aspects, the invention provides delivery systems for delivery of peptides in the CNS. In some embodiments, the delivery system comprises (1) a CA4 binding peptide having specificity to primate CA4 and/or human CA4; and (2) an agent. In some embodiments, the CA4 binding peptide is (1) displayed on the surface of the delivery system; or (2) partially embedded in the delivery system. In some embodiments, the delivery system is selected from the group consisting of: nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a viral vector or a non-viral vector. In some embodiments, the delivery system comprises a nanoparticle selected from the group consisting of: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

In some embodiments, the delivery system comprises a viral vector or a non-viral vector. For example, the viral vector can comprise an adenovirus vector, an adeno-associated virus (AAV) vector, a lentiviral vector, or a retrovirus vector. In some embodiments, the viral vector is an AAV vector and the target peptide can be part of a capsid protein of the AAV vector.

Adeno-Associated Virus (AAV) and Recombinant AAV (rAAV)

In some embodiments, the delivery system for delivering a payload across the BBB is an AAV vector. In some embodiments, the AAV is a replication-deficient parvovirus, the single-stranded DNA genome of which is about 4.7 kb in length including 145 nucleotide inverted terminal repeats (ITRs). The ITRs play a role in the integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for the production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

In some embodiments, the AAV vectors can comprise coding regions of one or more proteins of interest. The AAV vector can include a 5′ AAV ITR, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulator-element downstream of the restriction site and upstream of the 3′ AAV ITR.

The viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid protein can be derived from an AAV serotype selected from AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV968, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3. l/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145 1/hu.53, AAV145.5/hu.54, AAV145.6/hu 55, AAV161. 10/hu.60, AAV161 6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu 3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu 44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh 61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu 43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-1O, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The AAV vector can be an AAV9 having an amino acid sequence provided in Table 2 or an amino acid sequence having at least 70% sequence identity (e.g., at least 70%, 80%, 90%, 95%, 96%, 97%, 98%, 99% or higher) to an amino acid sequence provided in Table 2. In some embodiments, the AAV vector is a variant AAV vector having at least 70% sequence identity (e.g., at least 70%, 80%, 90%, 95%, 96%, 97%, 98%, 99% or higher) to an amino acid sequence of any known AAV9 variant.

In some embodiments, the AAV vectors disclosed herein can be used as AAV transfer vectors carrying a transgene encoding a protein of interest (e.g., a targeting peptide) for producing recombinant AAV viruses that can express the protein of interest in a host cell. Accordingly, disclosed herein also include recombinant AAV viruses (rAAV). The rAAV can comprise an AAV capsid protein described herein.

The rAAV can comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV can be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype ban be pseudotyped, which means that the ITRs from a first AAV serotype (e.g., AAV1) are used in a capsid from a second AAV serotype (e.g., AAV9), wherein the first and second AAV serotypes are not the same. As a non-limiting example, a pseudotyped AAV serotype comprising the AAV1 ITRs and AAV9 capsid protein may be indicated AAV1/9. The rAAV may additionally, or alternatively, comprise a capsid that has been engineered to express an exogenous ligand binding moiety (e.g., receptor), or a native receptor that is modified.

In some embodiments, the rAAV capsid proteins comprise a substitution or insertion of one or more amino acids in an amino acid sequence of an AAV capsid protein. The rAAV capsid proteins described herein have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45-555-564 (1983); the complete genome of AAV-3 is provided in GenBank Accession No. NC_1829, the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76(2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

In some embodiments, the rAAV vectors disclosed herein can carry a transgene encoding a CA4 binding peptide described herein that is capable of binding human CA4 and/or primate CA4. The targeting peptide can be part of a capsid of the rAAV. Disclosed herein also include AAV capsid proteins. The AAV capsid protein can comprise a targeting peptide disclosed herein.

The location of the targeting peptide within the capsid protein can vary. In some embodiments, the targeting peptide can be inserted between two adjacent amino acids in AA586-595 (e.g., between AA586 and AA587, AA587 and AA588, AA588 and AA589, AA589 and AA590, AA590 and AA591, AA591 and AA592, AA592 and AA593, AA593, AA594 and AA595) of AAV9 capsid protein or functional equivalents thereof in other AAV capsid proteins. The AAV vector can be a vector selected from AAV1, AAV2, AAV3, AAV3b, AAV4, AAV5, AAV6, AAV7. AAV8, AAV9, AAV10, AAV-DJ, human isolate hu.31, human isolate hu.32, rhesus isolate rh.8, rhesus isolate rh.10, or a variant thereof. In some embodiments, the AAV vector is AAV9 or a variant or a derivative thereof. For example, the AAV capsid protein comprises, or consists thereof, provided in Table 2, or an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence provided in Table 2.

The targeting peptide can be inserted between AA588-589 of AAV9 capsid protein or functional equivalents thereof in other AAV capsid proteins. The two adjacent amino acids can be AA588-589. In some embodiments, the targeting peptide is inserted between AA587-590 of AAV9 capsid protein or functional equivalents thereof in other AAV capsid proteins.

Uses of AAV Vectors and rAAVs for Payload Delivery

In certain aspects, the invention provides compositions for use in the delivery of a payload (e.g., a pharmaceutical agent) to a target environment such as a nervous system of a subject. The composition can comprise an AAV comprising (1) an AAV capsid protein disclosed herein and (2) an agent to be delivered to a target environment (e.g., nervous system) of the subject.

The target environment can be the CNS, the peripheral nervous system (PNS), or a combination thereof. The target environment can be brain endothelial cells, neurons, capillaries in the brain, arterioles in the brain, arteries in the brain, or a combination thereof. In certain embodiments, the target environment may be in the eye of a primate or human.

The pharmaceutical agent to be delivered can comprise a nucleic acid, a peptide, a small molecule, an aptamer, or a combination thereof. The AAV vectors disclosed herein can be effectively transduced to a target environment (e.g., the CNS), for example, for delivering nucleic acids. In some embodiments, a method of delivering a nucleic acid sequence to the nervous system is provided. The protein can be part of a capsid of an AAV. The AAV can comprise a nucleic acid sequence to be delivered to a nervous system. One can then administer the AAV to the subject.

The nucleic acid sequence to be delivered to a nervous system can comprise one or more of: a) a sequence encoding a trophic factor, a growth factor, or other soluble factors that might be released from the transduced cells and affect the survival or function of that cell and/or surrounding cells; b) a DNA (e.g., genomic or cDNA sequence) that restores protein function to humans or animals harboring a genetic mutation(s) in that gene; c) a DNA that encodes a protein that can be used to control or alter the activity or state of a cell: d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

In some embodiments, the vector can also comprise regulatory control elements known to one of skill in the art to influence the expression of the RNA and/or protein products encoded by the polynucleotide within desired cells of the subject.

Functionally, expression of the polynucleotide can be at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of the intron. Another example of a regulatory element is a transcription termination signal and/or a polyadenylation sequence.

Expression control elements and promoters include those active in a particular tissue or cell type, referred to herein as a “tissue-specific expression control elements/promoters” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

In certain preferred aspects, the invention provides methods of treatment or diagnostic analysis of conditions related to CNS and/or eye by administration compositions comprising the peptides or AAVs provided herein. In certain embodiments, the peptides and/or AAVs of the invention allow for the delivery of therapeutic cargo directed to treatment of CNS and/or eye across the BBB. In certain embodiments, the invention provides methods of treatment of brain disorders. In certain embodiments, the brain disorders treated by the compositions of the invention include brain cancer, neurodegeneration and glioblastoma. In certain embodiments, the condition associated with eye is glaucoma.

In certain embodiments, the invention provides compositions for use in the delivery of an agent to a nervous system of a subject in need. In some embodiments, the composition comprises an AAV comprising (1) an AAV capsid protein disclosed herein and (2) an agent to be delivered to the nervous system of the subject; optionally wherein the nervous system is the central nervous system (CNS), the peripheral nervous system (PNS), or a combination thereof. In some embodiments, the nervous system is brain endothelial cells, neurons, capillaries in the brain, arterioles in the brain, arteries in the brain, or a combination thereof. In some embodiments, the composition is a pharmaceutical composition comprising one or more pharmaceutical acceptable carriers. In some embodiments, the agent to be delivered comprises a nucleic acid, a peptide, a small molecule, an aptamer, or a combination thereof.

The nucleic acid (e.g., a heterologous nucleic acid) can comprise a 5′ ITR and a 3′ ITR. The agent can comprise a DNA sequence encoding a protein (e.g., atrophic factor, a growth factor, or a soluble protein). The nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. In some embodiments, the silencing effector comprises one or more miRNA binding sites (e.g., miR-122 binding sites). miRNA binding sites are operably linked regulatory elements that are typically located in the 3′UTR of the transcribed sequence. Binding of miRNAs to the target transcript (in complex with the RNA-Induced Silencing Complex, RISC) can reduce the expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. In some embodiments, the gene of interest can be selected from SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. The nucleic acid further can comprise a polynucleotide encoding one or more secondary proteins, and the protein and the one or more secondary proteins can comprise a synthetic protein circuit. The nucleic acid can comprise a single-stranded AAV (ssAAV) vector or a self-complementary AAV (scAAV) vector.

The promoter can comprise a ubiquitous promoter. The ubiquitous promoter can be selected from a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, e.g. a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha 1 promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be, or comprise, a muscle-specific promoter, e.g., an MCK promoter.

The promoter can comprise an intronic sequence. The promoter can comprise a bidirectional promoter and/or an enhancer. In some embodiments, the enhancer can be a CMV enhancer. One or more cells of a subject can comprise an endogenous version of a nucleic acid sequence (e.g., a gene), and the promoter can comprise or can be derived from the promoter of the endogenous version. In some embodiments, one or more cells of a subject comprise an endogenous version of the nucleic acid sequence, and the sequence is not truncated relative to the endogenous version.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 1 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

The rAAV disclosed herein can comprise one or more of the nucleic acids disclosed herein. The nucleic acid can comprise a polynucleotide encoding a protein. The nucleic acid can be or can encode an RNA agent. The nucleic acid can comprise a promoter operably linked to the polynucleotide encoding a protein. As disclosed herein, the gene is operatively linked with appropriate regulatory elements in some embodiments. The one or more genes of the nucleic acid can comprise an siRNA, an shRNA, an antisense RNA oligonucleotide, an antisense miRNA, a trans-splicing RNA, a guide RNA, a single-guide RNA, a crRNA, a tracrRNA, a trans-splicing RNA, a pre-mRNA, an mRNA, or any combination thereof. The one or more genes of the nucleic acid can comprise one or more synthetic protein circuit components. The one or more genes of the nucleic acid can comprise can entire synthetic protein circuit comprising one or more synthetic protein circuit components. The one or more genes of the nucleic acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof, insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof.

### Pharmaceutical Compositions:

In certain aspects, the invention provides administration of composition comprising the peptides provided herein in a subject. In certain embodiments, the compositions of the invention are administered as systemic bloodstream delivery. In certain embodiments, the compositions of the invention are administered as local direct injections. In certain embodiments, the invention provides compositions comprising AAVs and/or peptides of the invention. In certain embodiments, the compositions are injectables. In certain embodiments, the compositions are ointments. In certain embodiments, the compositions are orally administered compositions. In certain embodiments, the compositions are orally administered compositions.

Also disclosed herein are pharmaceutical compositions comprising one or more of the rAAV viruses (or other delivery systems) disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol, salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include methods of delivering an agent to a nervous system of a subject. In some embodiments, the method comprises: providing an AAV vector comprising an AAV capsid protein disclosed herein. In some embodiments, the AAV vector comprises an agent to be delivered to the nervous system. In some embodiments, the method comprises administering the AAV vector to the subject. The composition can be for intravenous administration. The composition can be for systemic administration. In certain embodiments, the invention provides local administration of the pharmaceutical composition. The subject can be an adult animal.

Titers of the rAAV to be administered will vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration will vary depending upon the age, weight, severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response is measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g., individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure are administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations can be converted to vg or viral genomes per kg or into total viral genomes administered by one of skill in the art.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The administration can be a systemic administration. The administration can be an intravenous administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. In some embodiments, the recombinant virus is administered to the subject by systematic transduction. In some embodiments, the recombinant virus is administered to the subject by intramuscular injection. In some embodiments, the rAAV is administered to the subject by the parenteral route (e.g., by intravenous, intramuscular or subcutaneous injection), by surface scarification or by inoculation into a body cavity of the subject Route(s) of administration and serotype(s) of AAV components of the rAAV virus can be readily determined by one skilled in the art taking into account the infection and/or disease state being treated and the target cells/tissue(s) that are to express the protein of interest. In some embodiments, it can be advantageous to administer the rAAV via intravenous administration. The variant AAV provided herein can advantageously provide for intravenous administration of vectors with enhanced tropisms for CNS.

In some embodiments, the subject is a primate and the agent is delivered to the endothelial cells and/or neurons of the nervous system. The nervous system can be the central nervous system (CNS). The agent can be delivered to the endothelial cells of the nervous system of the subject at least 1.5-fold, 2-fold, or 3-fold more efficiently than the delivery of the agent to the neurons of the nervous system. In some embodiments, the agent is delivered to the endothelial cells of the nervous system of the subject more than 3-fold more efficiently (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the neurons of the nervous system.

Disclosed herein include methods of delivering an agent (e.g., a therapeutic agent) to a cell. In some embodiments, the method comprises: contacting an AAV vector comprising an AAV capsid protein disclosed herein with the cell. In some embodiments, the AAV vector comprises an agent to be delivered to the nervous system. In some embodiments, the cell is an endothelial cell or a neuron. In some embodiments, contacting the AAV vector with the cell occurs in vitro, in vivo or ex vivo. The cell can be present in a tissue, an organ, or a subject. The cell can be a brain endothelial cell, a neuron, a cell in the capillaries in the brain, a cell in the arterioles in the brain, a cell in the arteries in the brain, a cell in the brain vasculature, or a combination thereof.

The AAV vector can be an AAV9 vector, or a variant thereof. In some embodiments, the AAV vector is a vector selected from AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

The variant AAV capsid can comprise tropism for a tissue or a cell of a central nervous system (CNS). The target cell can be a neuronal cell, a neural stem cell, an astrocyte, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell.

Actual administration of the rAAV can be accomplished by using any physical method that will transport the rAAV into the nervous system of the subject. For example, the rAAV disclosed herein can advantageously be administered intravenously for delivery to the CNS. As disclosed herein, capsid proteins of the rAAV can be modified so that the rAAV is targeted to a particular target environment of interest such as central nervous system, and to enhance tropism to the target environment of interest (e.g., CNS tropism). Pharmaceutical compositions can be prepared, for example, as injectable formulations.

A therapeutically effective amount of the rAAV can be administered to a subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with CNS, which includes but is not limited to Krabbe disease, Sandhoff disease, Tay-Sachs, Gaucher disease (Type I, II, or III), Niemann-Pick disease (NPC1 or NPC2 deficiency), Hurler syndrome, Pompe disease, Batten disease, or any combination thereof), chronic pain, or a combination thereof. In some embodiments, the rAAV is administered to the subject during remission of the disease or disorder. In some embodiments, the rAAV is administered prior to the onset of the disease or disorder in the subject. In some embodiments, the rAAV is administered to a subject at a risk of developing the disease or disorder.

Disclosed herein, in some embodiments, are formulations of pharmaceutically-acceptable excipients and carrier solutions suitable for delivery of the compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. In some embodiments, the rAAV disclosed herein can advantageously be administered intravenously for delivery to the CNS.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the compositions disclosed herein (e.g., rAAV compositions), which are prepared by incorporating the compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

In certain embodiments, the invention provides kits comprising compositions disclosed herein. Also disclosed herein are kits for the treatment or prevention of a disease or conditions of the CNS, PNS, or target organ or environment (e.g., CNS). In some instances, the disease or condition is cancer, a pathogen infection, neurological disease, muscular disease, or an immune disorder, such as those described herein. In one embodiment, a kit can include a therapeutic or prophylactic composition containing an effective amount of a composition of a rA AV particle encapsidating a nucleic acid provided herein and a rAAV capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, rAAV are provided together with instructions for administering the rAAV to a subject having or at risk of developing the disease or condition. Instructions can generally include information about the use of the composition for the treatment or prevention of the disease or condition.

The kit can include allogenic cells. In some embodiments, a kit includes cells that can comprise a genomic modification. In some embodiments, a kit comprises “off-the-shelf” cells. In some embodiments, a kit includes cells that can be expanded for clinical use. In some embodiments, a kit contains contents for a research purpose.

In some embodiments, the instructions include at least one of the following: description of the therapeutic rAAV composition; dosage schedule and administration for treatment or prevention of the disease or condition disclosed herein; precautions; warnings; indications; counter-indications: overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some embodiments, instructions provide procedures for administering the rAAV to the subject alone. In some embodiments, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), pnon related infections, diseases involving mitochondrial dysfunction, diseases involving P-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

In certain preferred embodiments, the invention provides a method of treatment of disorder of the brain. In certain preferred embodiments, the disorder of brain is brain cancer. In certain other preferred embodiments, the invention provides a method of treatment of a disorder related to the eye. In certain preferred embodiments, the disorder related to eye is glaucoma. In certain embodiments, the invention provides that disorders of brain and/or eye are treated by administration of compositions comprising CA4 binding peptides or CA4 binding AAVs of the invention.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A carbonic anhydrase IV-binding peptide selected from the group consisting of the carbonic anhydrase IV-binding peptides listed in Tables 1, 3, 4, and 5.

2. The carbonic anhydrase IV-binding peptide of claim 1, wherein the carbonic anhydrase IV-binding peptide is selected from the group consisting of 9-mer carbonic anhydrase IV-binding peptides listed in Tables 4 and 5.

3. An AAV capsid protein comprising a carbonic anhydrase IV-binding peptide of claim 1 or 2.

4. The AAV capsid protein of claim 3, wherein the AAV capsid protein comprises AAV9 as a parent sequence.

5. An AAV capsid comprising an AAV capsid protein of claim 3 or 4.

6. The AAV capsid of claim 5, wherein the AAV capsid further comprises a therapeutic cargo or diagnostic cargo to be delivered to the central nervous system (CNS) of a primate or a human.

7. The AAV capsid of claim 6, wherein the therapeutic cargo or diagnostic cargo is delivered to therapeutic targets in the CNS after crossing blood-brain barrier (BBB).

8. The AAV capsid of claim 5 or 6, wherein the therapeutic cargo is selected from the group consisting of nucleic acids, antibodies, peptides, and small molecules.

9. The AAV capsid protein of claim 3, wherein the AAV capsid protein is Alpha 43, 45, 48 and 49, listed in table 1.

10. The AAV capsid protein of claim 9, wherein the AAV capsid protein binds to human CA4.

11. The AAV capsid protein of claim 9 or 10, wherein the AAV capsid proteins comprise peptides selected from the group consisting of: DGVVHETVR (SEQ ID NO: 7); DGVVGVNIR (SEQ ID NO: 8); DGEVGLTVR (SEQ ID NO: 9); DGIVGSTIR (SEQ ID NO. 10).

12. A composition comprising carbonic anhydrase IV-binding peptide, an AAV capsid protein, and/or an AAV of claims 1-11.

13. The composition of claim 12, wherein the composition is an injectable formulation.

14. The composition of claim 13, wherein the composition further comprises a pharmaceutically acceptable excipient.

15. A method for treatment or diagnosis of a condition by administration of a composition comprising a carbonic anhydrase IV-binding peptide, an AAV capsid protein, and/or an AAV of claims 1-11.

16. The method of claim 15, wherein the condition is brain disorder or an eye disease.

17. The method of claim 16, wherein the brain disorder is selected from the group consisting of brain cancer, neurodegeneration and glioblastoma.

18. The method of claim 16, wherein the eye disease is glaucoma.

19. The method of claim 15, wherein the composition is administered by systemic bloodstream delivery.

20. The method of claim 15, wherein the composition is administered by a local injection.
